(19) United States
(12) Patent Application Publication
(10) Pub. No.: US 2025/0003877 A1
(43) Pub. Date: Jan. 2, 2025

(54) Silyl Bridged Dyes

(71) Applicant: University of Massachusetts, Boston, MA (US)

(72) Inventors: Stephen Miller, Cambridge, MA (US); Nageswara Rao Desaboini, Wilmington, DE (US)

(21) Appl. No.: 18/700,599

(22) PCT Filed: Oct. 12, 2022
(86) PCT No.: PCT/US2022/077941

§ 371 Date: Apr. 11, 2024

Related U.S. Application Data
(60) Provisional application No. 63/262,443, filed on Oct. 12, 2021.

(51) Int. Cl. G01N 21/64; C09B 69/00; C09K 11/06; G01N 33/58

(52) U.S. Cl. CPC G01N 21/6428; C09B 69/008; C09K 11/06; G01N 21/6456; G01N 33/582; C09K 2211/1007; C09K 2211/1014; C09K 2211/1022; G01N 2021/6439

(57) ABSTRACT

Provided herein are fluorescent dyes possessing abroad range of silyl modifications, which can be used, for example, for enabling brighter dyes, red-shifting of fluorescence, and as handles, e.g., for the introduction of sensors and/or targeting groups, including no-wash fluorogenic labeling agents for nuclear DNA, SnapTag® proteins, and HaloTag® proteins. The fluorescent dyes have the following structure: or a tautomer thereof, or a salt of the foregoing, wherein values for the variables (e.g., X, Z¹, Z², R³, R⁶, R⁷, R¹¹) are as described herein.

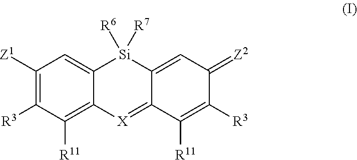

### RELATED APPLICATION

This application claims the benefit of U.S. Provisional Application No. 63/262,443, filed on Oct. 12, 2021. The entire teachings of this application are incorporated herein by reference.

### STATEMENT OF GOVERNMENT SUPPORT

This invention was made with government support under grant number GM135474 awarded by the National Institutes of Health. The government has certain rights in the invention.

### BACKGROUND

Fluorescent dyes such as rhodamines are widely used to assay the activity and image the location of otherwise invisible molecules. Si-rhodamines, in which the bridging oxygen of rhodamines is replaced with a dimethylsilyl group, are increasingly the dye scaffold of choice for biological applications, as fluorescence is red-shifted by 70-100 nm while maintaining high brightness, and this family of dyes has demonstrated utility in super-resolution imaging and single-molecule imaging experiments.

Siloles have long been known to exhibit red-shifted fluorescence properties compared to their analogous silicon-free heterocycles due to conjugation between the Si σ* orbitals and the π* orbitals of the chromophore (Yamaguchi et. al. Modification of the Electronic Structure of Silole by the Substituents on the Ring Silicon. Journal of Organometallic Chemistry 1998, 559 (1), 73-80). However, it wasn't until 2008 that this LUMO-lowering effect was shown to apply to long-wavelength xanthene dyes (Fu et al. A Design Concept of Long-Wavelength Fluorescent Analogs of Rhodamine Dyes: Replacement of Oxygen with Silicon Atom. Chem. Commun. 2008, No. 15, 1780-1782). Si-rhodamine dyes have been widely studied and optimized ever since, and the effect of this modification has recently been shown to extend to other classes of near-IR dye scaffolds (Choi et al. Silicon Substitution in Oxazine Dyes Yields Near-Infrared Azasiline Fluorophores That Absorb and Emit beyond 700 Nm. Org. Lett. 2018, 20 (15), 4482-4485, and Pengshung et al. Silicon Incorporation in Polymethine Dyes. Chemical Communications 2020, 56 (45), 6110-6113).

Despite this intense interest, virtually all Si-rhodamines reported thus far have been confined to dimethylsilyl substitution. The dimethylsilyl group has been ubiquitous among the Si-rhodamines reported to date presumably because of its small size. However, more extensive modification of the silyl group—something that is not possible with oxygen-bridged rhodamines—represents a missed opportunity.

Accordingly, there is a need for Si-containing dyes having a variety of silyl modifications.

### SUMMARY

The present disclosure provides Si-containing dyes having a variety of silyl modifications that can be used, for example, to tune dye behavior or tether a dye to a sensor or biomolecule for imaging applications. The compounds described herein can be used to label biologically relevant materials such as antibodies, peptides, and nucleic acids, and/or can serve as useful markers for fluorescence imaging and spectroscopy.

One aspect of the present disclosure provides a compound having the following structural formula:

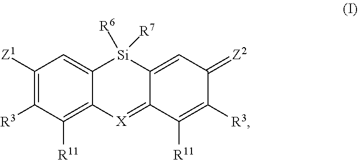

or a tautomer thereof, or a salt of the foregoing, wherein values for the variables (e.g., X, Z¹, Z², Ar, R³, R⁶, R⁷, R¹¹) are as described herein.

Another aspect of the present disclosure provides a method of modifying a compound of Structural Formula I, or a tautomer thereof, or a salt of the foregoing, wherein R⁶ is (C₂-C₂₅)aliphatic or (C₂-C₂₅)heteroaliphatic substituted with a leaving group and values for the remaining variables are as described herein. The method comprises reacting the compound of Structural Formula I, or a tautomer thereof, or a salt of the foregoing, or an appropriately protected derivative of any of the foregoing, with a nucleophile under conditions suitable for the nucleophile to displace the leaving group.

Another aspect of the present disclosure provides a method of imaging a cell (e.g., a live cell), comprising contacting the cell with a compound of Structural Formula (I), or a tautomer thereof (e.g., a ring-closed tautomer thereof), or a salt of the foregoing; illuminating the cell; and detecting fluorescence from the cell.

Yet another aspect of the present disclosure provides a method of detecting a target in a sample, comprising contacting the sample with a compound of Structural Formula (I) comprising a targeting group for the target, or a tautomer thereof (e.g., a ring-closed tautomer thereof), or a salt of the foregoing; illuminating the sample; and detecting fluorescence from the sample.

Another aspect of the present disclosure provides a method of labeling a biomolecule or cell (e.g., in a multicellular organism), comprising contacting the biomolecule or cell with a compound of Structural Formula (I), or a tautomer thereof (e.g., a ring-closed tautomer thereof), or a salt of the foregoing.

Another aspect of the present disclosure provides a use of a compound of Structural Formula (I) (e.g., a compound of Structural Formula (I) comprising a targeting group for the target), or a tautomer thereof (e.g., a ring-closed tautomer thereof), or a salt of the foregoing, for example, for imaging a cell (e.g., a live cell), detecting a target in a sample or labeling a biomolecule or cell (e.g., in a multicellular organism).

The silyl modifications disclosed herein provide fluorescent dyes that are brighter and red-shifted compared to their dimethylsilyl counterparts, and contain sensors and/or handles for further functionalization, e.g., for use as no-wash fluorogenic labeling agents for nuclear DNA and/or HaloTag® labeling. For example, diphenyl and divinyl Si-rhodamines are red-shifted by 10-15 nm compared to their dimethylsilyl counterparts, dioctyl substitution renders dyes more hydrophobic, and vinyl and chloropropyl silyl dyes include functional handles that could be used for further elaboration, e.g., into iodides, clickable azides and/or functionalized thioethers. Additionally, molecular sensors and biomolecular targeting groups can be directly incorporated into the silyl bridge, enabling new ways of modulating fluorescence and applications such as no-wash labeling of the nucleus and targeted fusion proteins. For example, molecular sensors and biomolecular targeting groups directly incorporated into the silyl bridge of a Si-rhodamine enabled no-wash labeling of, for example, the nucleus, SnapTag®, and HaloTag® proteins.

Specific examples of Si bridge modifications to tune and functionalize Si dyes disclosed herein include the following handles/targeting groups: carboxylates, iodides and N-hydroxysuccinimide (NHS) esters, clickable azides and norbornenes, amine (O2) HaloTag®, SnapTag® and Hoechst 33258.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIG. 1 is an x-ray crystal structure of compound 037, and shows that compound 037 is the “s,s” isomer, where the silyl chloropropyl group and the phenyl of the spirolactone are on opposite faces of the planar chromophore.

FIG. 2A shows images of dye fluorescence and dye fluorescence overlaid with DIC from no-wash imaging of live HeLa cells with SiR-DNA.

FIG. 2B shows images of dye fluorescence and dye fluorescence overlaid with DIC from no-wash imaging of live HeLa cells with compound 061.

FIG. 2C shows images of dye fluorescence and dye fluorescence overlaid with DIC from no-wash imaging of live HeLa cells with compound 062.

FIG. 3A shows no-wash imaging, including dye fluorescence, GFP, and overlay with DIC, of HaloTag®-GFP-expressing HeLa cells with 200 nM dye with compound 049.

FIG. 3B shows no-wash imaging of HaloTag®-GFP-expressing HeLa cells with 200 nM dye with JF646-HaloTag® ligand.

FIG. 3C shows no-wash imaging, including dye fluorescence, GFP, and overlay with DIC, of HaloTag®-GFP expressing HeLa cells with 200 nM dye with compound 050.

FIG. 3D shows no-wash imaging, including dye fluorescence, GFP, and overlay with DIC, of HaloTag®-GFP-expressing HeLa cells with 200 nM dye with compound 051.

FIG. 3E shows no-wash imaging, including dye fluorescence, GFP, and overlay with DIC, of HaloTag®-GFP-expressing HeLa cells with 200 nM dye with compound 044.

FIG. 3F shows no-wash imaging, including dye fluorescence, GFP, and overlay with DIC, of HaloTag®-GFP-expressing HeLa cells with 200 nM dye with compound 045.

FIG. 3G shows no-wash live cell imaging of HeLa cells transfected with pHaloTag-EGFP with 200 nM dye with compound 068.

FIG. 4A shows HaloTag®-GFP fluorescence in the GFP channel.

FIG. 4B shows HaloTag®-GFP labeling with compound 045 in the Cy5 channel.

FIG. 4C shows HaloTag®-GFP labeling with compound 045 fluorescence colocalization in the GFP and Cy5 channels and DIC.

FIG. 5A shows pSNAPf-H2B labeling with 3 μM SNAP-Cell® 647 SiR. Scale bar is 20 μm.

FIG. 5B shows pSNAPf-H2B labeling with 3 μM compound 065. Scale bar is 20 μm.

FIG. 5C shows pSNAPf-H2B labeling with 3 μM compound 066. Scale bar is 20 μm.

### DETAILED DESCRIPTION

### Definitions

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

The terms “a,” “an,” “the” and similar terms used in the context of the present disclosure (especially in the context of the claims) are to be construed to cover both the singular and plural unless otherwise indicated herein or clearly contradicted by the context.

“Aliphatic,” as used herein, refers to a non-aromatic, branched, straight-chain and/or cyclic, hydrocarbon radical having the specified number of carbon atoms. Thus, “(C₁-C₁₀)aliphatic” refers to an aliphatic radical having from one to 10 carbon atoms. In some embodiments, aliphatic is (C₁-C₂₅)aliphatic, for example, (C₁-C₁₁)aliphatic, (C₁-C₁₀)aliphatic, (C₁-C₆)aliphatic, (C₁-C₅)aliphatic or (C₁-C₃)aliphatic. “Aliphatic” can be saturated or contain one or more units of unsaturation. Examples of aliphatic include alkyl, alkenyl and alkynyl. In some embodiments, aliphatic is alkyl, alkenyl or alkynyl. In some aspects, aliphatic is alkyl. In some embodiments, aliphatic is cyclic, for example, (C₃-C₁₂)cycloaliphatic, (C₃-C₈)cycloaliphatic or (C₃-C₆)cycloaliphatic. In some embodiments, aliphatic is cycloalkyl, for example, (C₃-C₁₂)cycloalkyl, (C₃-C₈)cycloalkyl or (C₃-C₆)cycloalkyl. In some embodiments, aliphatic is cycloalkenyl, for example, (C₃-C₁₂)cycloalkenyl, (C₃-C₈)cycloalkenyl or (C₃-C₆)cycloalkenyl. In some embodiments, aliphatic is cycloalkynyl, for example, (C₈-C₁₂)cycloalkynyl or (C₈)cycloalkynyl.

As used herein, the term “alkyl” refers to a branched or straight-chain, saturated, monovalent, hydrocarbon radical having the specified number of carbon atoms. Thus, the term “(C₁-C₆)alkyl” refers to a branched or straight-chain, saturated, monovalent, hydrocarbon radical having from one to six carbon atoms. Examples of alkyl include, but are not limited to, methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, and 2-methylpentyl.

The term “alkenyl” refers to a branched or straight-chain, monovalent, hydrocarbon radical having the specified number of carbon atoms and at least one (e.g., one, two, three, four, five, etc.) carbon-carbon double bond. Thus, the term “(C₁-C₆)alkenyl” refers to a branched or straight-chain, monovalent, hydrocarbon radical having from one to six carbon atoms and at least one carbon-carbon double bond. Examples of alkenyl include, but are not limited to, ethenyl, vinyl, allyl, octenyl, decenyl.

The term “alkynyl” refers to a branched or straight-chain, monovalent, hydrocarbon radical having the specified number of carbon atoms and at least one (e.g., one, two, three, four, five, etc.) carbon-carbon triple bond. Thus, the term “(C₁-C₆)alkynyl” refers to a branched or straight-chain, monovalent, hydrocarbon radical having from one to six carbon atoms and at least one carbon-carbon triple bond. Examples of alkynyl include, but are not limited to, acetylenyl, ethynyl, n-propynyl, isopropynyl, n-butynyl, isobutynyl, t-butynyl, octynyl, decynyl. “Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Aryl” refers to a monocyclic or polycyclic (e.g., bicyclic, tricyclic), carbocyclic, aromatic ring system having the specified number of ring atoms, and includes aromatic rings fused to non-aromatic rings, as long as one of the fused rings is an aromatic hydrocarbon. Thus, “(C₆-C₁₅)aryl” means an aromatic ring system having from 6-15 ring atoms. Examples of aryl include phenyl, naphthyl and fluorenyl.

The term “cycloalkyl,” as used herein, refers to a saturated, monocyclic or polycyclic (e.g., bicyclic, tricyclic), aliphatic, hydrocarbon ring system having the specified number of carbon atoms. Thus, “(C₅-C₈)cycloalkyl” means a cycloalkyl ring system having from 5 to 8 ring carbons. Examples of cycloalkyl include, but are not limited to, cyclopropyl, cyclobutyl, cyclopentyl, cyclohexyl, bicyclo[2.2.1]heptanyl, bicyclo[2.2.2]octanyl, and norbornyl.

“Halogen” and “halo,” as used herein, refer to fluorine, chlorine, bromine or iodine. In some embodiments, halogen is fluoro, chloro or bromo. In some embodiments, halogen is fluoro or chloro. In some embodiments, halogen is chloro, bromo or iodo. In some embodiments, halogen is chloro or bromo.

“Halo,” as used herein, refers to fluoro, chloro, bromo or iodo. In some embodiments, halo is fluoro or chloro. In some embodiments, halo is chloro, bromo or iodo. In some embodiments, halo is bromo or iodo. In some embodiments, halo is fluoro, chloro or bromo.

“Haloalkyl,” as used herein, refers to an alkyl radical wherein one or more hydrogen atoms is each independently replaced by a halogen, wherein alkyl and halogen are as described herein. “Haloalkyl” includes mono-, poly- and perhaloalkyl groups. “(C₁-C₆)haloalkyl” refers to a (C₁-C₆)alkyl wherein one or more hydrogen atoms is each independently replaced by a halogen. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl.

“Haloalkoxy” refers to a haloalkyl radical attached through an oxygen linking atom, wherein haloalkyl is as described herein.

“Heteroatom” refers to an atom that is not carbon or hydrogen. Examples of heteroatoms include, but are not limited to, nitrogen, oxygen, sulfur, boron, silicon, and the like. In some embodiments, heteroatom is selected from nitrogen, oxygen and sulfur.

“Heteroaliphatic,” as used herein, refers to a non-aromatic, branched, straight-chain and/or cyclic, hydrocarbon radical having at least one carbon atom and the specified number of atoms in its chain and/or cycle, wherein at least one carbon atom in the chain and/or cycle has been replaced with a heteroatom (e.g., N, S, Si and/or O). Thus, “(C₂-C₁₀)heteroaliphatic” refers to a heteroaliphatic radical having from two to 10 atoms in its chain and/or cycle. In some embodiments, heteroaliphatic is (C₂-C₂₅)heteroaliphatic, for example, (C₂-C₁₅)heteroaliphatic, (C₂-C₁₀)heteroaliphatic, (C₂-C₆)heteroaliphatic, (C₂-C₅)heteroaliphatic or (C₂-C₃)heteroaliphatic. “Heteroaliphatic” can be saturated or contain one or more units of unsaturation. Examples of heteroaliphatic include heteroalkyl and heterocyclyl. In some embodiments, heteroaliphatic is heteroalkyl. In some embodiments, heteroaliphatic is cyclic, for example, (C₃-C₁₂)heterocycloaliphatic, (C₃-C₅)heterocycloaliphatic or (C₃-C₆)heterocycloaliphatic. In some embodiments, heteroaliphatic is heterocyclyl, for example, (C₃-C₁₂)heterocyclyl, (C₃-C₈)heterocyclyl or (C₃-C₆)heterocyclyl.

“Heterocyclyl” or “heterocycloalkyl” refers to an optionally substituted, saturated or unsaturated, non-aromatic, aliphatic, monocyclic or polycyclic (e.g., bicyclic, tricyclic), monovalent, hydrocarbon ring system having the specified number of ring atoms, wherein at least one carbon atom in the ring system has been replaced with a heteroatom. Thus, “(C₃-C₆)heterocyclyl” means a heterocyclic ring system having from 3-6 ring atoms. A heterocyclyl can be monocyclic, fused bicyclic, bridged bicyclic or polycyclic, but is typically monocyclic. A heterocyclyl can contain 1, 2, 3 or 4 (e.g., 1) heteroatoms independently selected from N, S and O. When one heteroatom is S, it can be optionally mono- or di-oxygenated (i.e., —S(O)— or —S(O)₂). A heterocyclyl can be saturated (i.e., contain no degree of unsaturation). Examples of monocyclic heterocyclyls include, but are not limited to, aziridine, azetidine, pyrrolidine, piperidine, piperazine, azepane, tetrahydrofuran, tetrahydropyran, morpholine, thiomorpholine, dioxide, oxirane.

The term “heteroaryl,” as used herein, refers to a monocyclic or polycyclic, aromatic, hydrocarbon ring system having the specified number of ring atoms, wherein at least one carbon atom in the ring has been replaced with a heteroatom. Thus, “(C₅-C₆)heteroaryl” refers to a heteroaryl ring system having five or six ring atoms. In some embodiments, heteroaryl has 5 to 15, 5 to 10, 5 to 9, or 5 to 6 ring atoms. A heteroaryl ring system may consist of a single ring or a fused ring system. A typical monocyclic heteroaryl is a 5- to 6-membered ring containing one to three heteroatoms (e.g., one, two or three) independently selected from oxygen, sulfur and nitrogen, and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a heteroaryl ring fused to an aryl ring (e.g., phenyl). Examples of heteroaryl include, but are not limited to, pyrrolyl, pyridyl, pyrazolyl, indolyl, indolinyl, isoindolinyl, indazolyl, thienyl, furanyl, benzofuranyl, dihydrobenzofuranyl, dihydroisobenzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, tetrahydroquinolinyl, benzofuranyl, benzopyranyl, benzothiophenyl, benzoimidazolyl, benzoxazolyl, 1H-benzo[d][1,2,3]triazolyl, and the like.

“Amino” refers to —NH₂.

“Alkylamino” refers to —N(H)(alkyl), wherein alkyl is as described herein. Examples of alkylamino include, but are not limited to, methylamino and ethylamino.

“Dialkylamino” refers to —N(alkyl)₂, wherein alkyl is as described herein. Each alkyl in a “dialkylamino” can be independently chosen, such that each alkyl in a dialkylamino can be the same or the alkyls in a dialkylamino can be different from one another.

“Cyclic amino” refers to a cyclic, aliphatic, monovalent, monocyclic or polycyclic, hydrocarbon ring radical having the specified number of ring atoms, wherein at least one carbon atom (e.g., one, two, three) has been replaced with a N. Thus, “(C₃-C₈)cyclic amino” means a cyclic amino ring radical having from 3-8 ring atoms. In some embodiments, one carbon atom in the ring system of a cyclic amino has been replaced with a N.

“Silacycle” refers to a cyclic aliphatic or heteroaliphatic ring system having the specified number of ring atoms containing at least one (e.g., one) silicon atom. Thus, “(C₅-C₈)silacycle” means a silacyclic ring system having from 5-8 ring atoms. A silacycle can be monocyclic, spirocyclic, fused bicyclic, bridged bicyclic or polycyclic. A silacycle can contain 1, 2, 3 or 4 (e.g., 1) silicon atoms. A silacycle can be saturated (i.e., contain no degree of unsaturation) or unsaturated.

As used herein, “sensor” refers to a molecule that undergoes a detectable change in response to a set of conditions, a species, a metal ion, etc. In fluorescence spectroscopy, the detectable change is typically a change in fluorescence, e.g., quenching/unquenching of fluorescence or a shift in the maximum wavelength of fluorescence. Non-limiting examples of sensors include spirolactonizable rhodamines, such as those disclosed herein, and sensors based on photoinduced electron transfer (PET), intramolecular charge transfer (ICT) and fluorescence resonance energy transfer (FRET). Other examples of sensors will be obvious to the skilled artisan, for example, based on the examples provided herein.

As used herein, “targeting group” refers to a molecule that binds to a biomolecule such as a protein or nucleic acid. Non-limiting examples of targeting groups include chlorotoxins (CTX), O⁶-benzylguanine, actin ligands such as jasplakinolide (e.g., in SiR-actin and MaP555-actin), microtubule ligands such as docetaxel (e.g., in SiR700-tubulin), Hoechst 33258, a nucleic acid stain, HaloTag® and SNAP-Tag® ligands. Other examples of targeting groups will be obvious to the skilled artisan, for example, based on the examples provided herein.

As used herein, “clickable moiety” refers to a functional group that is capable, under suitable conditions, of engaging in a click reaction. Examples of clickable moieties include azides, alkynes, phosphines, thiols, maleimides, isonitriles, and tetrazines.

As used herein, “click reaction” refers to a chemical reaction characterized by a large thermodynamic driving force that usually results in irreversible covalent bond formation. Click reactions can often be conducted in aqueous or physiological conditions without producing cytotoxic byproducts. Examples of click reactions include [3+2]cycloadditions, such as the Huisgen 1,3-dipolar cycloaddition reaction of an azide and an alkyne; thiol-ene reactions, such as the Michael addition of a thiol to a maleimide or other unsaturated acceptor; [4+1] cycloaddition reactions between an isonitrile and a tetrazine; the Staudinger ligation between an azide and an ester-functionalized phosphine or an alkanethiol-functionalized phosphine; Diels-Alder reactions (e.g., between a furan and a maleimide); and inverse electron demand Diels-Alder reactions (e.g., between a tetrazine and a dienophile such as a strained transcyclooctene or a norbornene).

The term “substituted,” as used herein, means that at least one (e.g., one, two, three, four, five, six, etc., such as from one to five, from one to three, one or two) hydrogen atom is replaced with a non-hydrogen substituent, provided that normal valencies are maintained and that the substitution results in a stable compound. Unless otherwise indicated, a “substituted” group can have a substituent at each substitutable position of the group. When more than one position in any given structure is substituted with more than one substituent selected from a specified group, the substituent can be the same or different at every position. An “optionally substituted group” can be substituted, as that term is described herein, or unsubstituted.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, or a 5-6-membered saturated, partially unsaturated, or aromatic ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aromatic mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂^{●}, -(haloR^{●}), —(CH₂)₀₋₂OH, —(CH₂)₀₋₂OR⁺, —(CH₂)₀₋₂CH(OR⁺)₂; —O(haloR*), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aromatic ring having 0-4 heteroatoms independently selected from nitrogen, oxygen, or sulfur. Such divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aromatic ring having 0-4 heteroatoms independently selected from nitrogen, oxygen, or sulfur. Divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aromatic ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aromatic ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aromatic ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aromatic mono- or bicyclic ring having 0-4 heteroatoms independently selected from nitrogen, oxygen, or sulfur.

Suitable substituents on the aliphatic group of RT are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aromatic ring having 0-4 heteroatoms independently selected from nitrogen, oxygen, or sulfur.

In a particular embodiment, suitable substituents are selected from —(CH₂)₀₋₄Ph (e.g., —CH₂Ph), which may be optionally substituted with halogen, —(CH₂)₀₋₂R^{●}, -(haloR^{●}), —(CH₂)₀₋₂OH, —(CH₂)₀₋₂OR^{●}, —O(haloR^{●}), —CN, —N₃, —(CH₂)₀₋₂SR^{●}, —(CH₂)₀₋₂SH or —NO₂, wherein each R* is unsubstituted or where preceded by “halo” is substituted only with one or more halogens, and is independently selected from C₁₋₄ aliphatic (e.g., C₁ aliphatic). In another embodiment, suitable substituents are selected from a protecting group or —(CH₂)₀₋₄Ph (e.g., —CH₂Ph), which may be optionally substituted with halogen, —(CH₂)₀₋₂R^{●}, -(haloR*), —(CH₂)₀₋₂OH, —(CH₂)₀₋₂OR^{●}, —O(haloR^{●}), —CN, —N₃, —(CH₂)₀₋₂SR^{●}, —(CH₂)₀₋₂SH or —NO₂, wherein each R^{●} is unsubstituted or where preceded by “halo” is substituted only with one or more halogens, and is independently selected from C₁₋₄ aliphatic (e.g., C₁ aliphatic).

In some embodiments, an optionally substituted group or compound, such as an optionally substituted aliphatic, is substituted with 0-5 (e.g., 0-3) substituents independently selected from oxo, halo, —CO₂H, —C(O)O—N-succinimide, maleimido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol, azido, propargyl, norbornenyl, or tetrazinyl, or a sensor or targeting group (e.g., oxo, halo, —CO₂H, —C(O)O—N-succinimide, maleimido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol, azido, propargyl, norbornenyl, or tetrazinyl). In some embodiments, an optionally substituted group or compound, such as an optionally substituted aliphatic, is substituted with 0-5 (e.g., 0-3) substituents independently selected from oxo, halo, —CO₂H, —C(O)O—N-succinimide, maleimido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol, azido or tetrazinyl, or a sensor or targeting group (e.g., oxo, halo, —CO₂H, —C(O)O—N-succinimide, maleimido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol, azido or tetrazinyl). In some embodiments, an optionally substituted group or compound, such as an optionally substituted aliphatic, is substituted with 0-5 (e.g., 0-3) substituents independently selected from oxo, halo, —CO₂H, —C(O)O—N-succinimide, maleimido, amino, hydroxyl, thiol, azido, propargyl, norbornenyl, or tetrazinyl, or a sensor or targeting group (e.g., oxo, halo, —CO₂H, —C(O)O—N-succinimide, maleimido, amino, hydroxyl, thiol, azido, propargyl, norbornenyl or tetrazinyl). In some embodiments, an optionally substituted group or compound, such as an optionally substituted aliphatic, is substituted with 0-5 (e.g., 0-3) substituents independently selected from oxo, halo, —CO₂H, —C(O)O—N-succinimide, maleimido, amino, hydroxyl, thiol, azido or tetrazinyl, or a sensor or targeting group (e.g., oxo, halo, —CO₂H, —C(O)O—N-succinimide, maleimido, amino, hydroxyl, thiol, azido or tetrazinyl). In some embodiments, an optionally substituted group or compound, such as an optionally substituted aryl or heteroaryl, is substituted with 0-5 (e.g., 0-3) substituents independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H.

Combinations of substituents and/or variables preferably result in stable compounds.

Unless specified otherwise, the term “compounds of the present disclosure” refers to a compound of any structural formula depicted herein (e.g., a compound of Structural Formula I, a subformula of a compound of Structural Formula I), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, e.g., pharmaceutically acceptable salts.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present invention.

When a disclosed compound is depicted by structure without indicating the stereochemistry, and the compound has one or more chiral centers, it is to be understood that the structure encompasses one enantiomer or diastereomer of the compound separated or substantially separated from the corresponding optical isomer(s), a racemic mixture of the compound, and mixtures enriched in one enantiomer or diastereomer relative to its corresponding optical isomer(s). When a disclosed compound is depicted by a structure indicating stereochemistry, and the compound has more than one chiral center, the stereochemistry indicates relative configuration of the substituents around the chiral centers. “R” and “S” can be used to indicate the absolute configuration of substituents around one or more chiral carbon atoms. D- and L- can also or alternatively be used to designate absolute stereochemistry.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)).

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)- isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

“Tautomer,” as used herein, refers to a structural isomer based on migration of an atom or group within a molecule. For example, a ketone (C(H)C(O)) group in a molecule may also exist as its tautomeric enol form (C═C(OH)). Compounds of the present disclosure, e.g., compounds of Structural Formula (I) wherein R⁴ or R⁵ is —P(O)OH(OR⁵⁰), —O—P(O)OH(OR⁵¹), SO₃H, —C(O)NH(R⁴⁰), (C₁-C₆)alkyl-OH, —OH, or —C(O)OH or where Q is appropriately substituted with —P(O)OH(OR⁵⁰), —O—P(O)OH(OR⁵¹), SO₃H, —C(O)NH(R⁴⁰), (C₁-C₆)alkyl-OH, —OH, or —C(O)OH, may also exist as their tautomeric spirocycles. An example of such a tautomeric pairing is shown below:

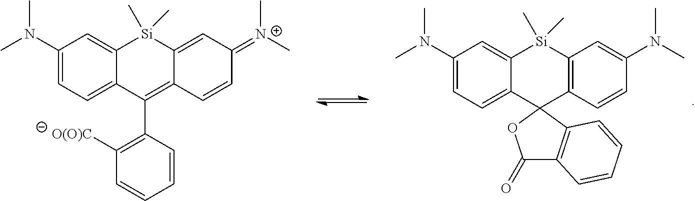

This disclosure is intended to cover all possible tautomers even when a structure depicts only one of them. Thus, this disclosure is intended to cover both the ring-opened carboxylate and the ring-closed spirolactone depicted above, even when a structure depicts only one of them.

When a compound of the present disclosure which may exist as its tautomeric spirocycle is depicted herein in its ring-opened tautomeric form, an “—O” designation is appended to the compound number. Thus, for example, in Table 1, Compound No. 035-0 denotes the ring-opened tautomer,

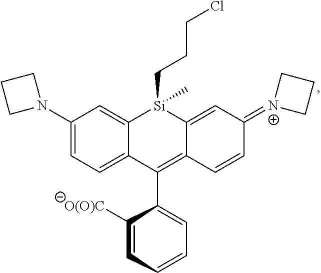

of Compound No. 035,

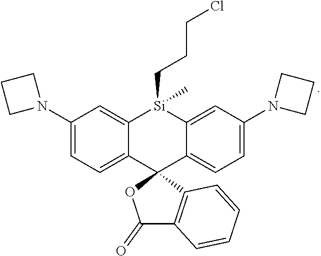 [SMILES: C[Si@]1(CCCCl)c2cc(N3CCC3)ccc2[C@@]2(OC(=O)c3ccccc32)c2ccc(N3CCC3)cc21]

It will be understood, however, that depiction of a single tautomer of a compound, e.g., of Compound No. 035, is intended to cover all possible tautomers of the compound, e.g., of Compound No. 035, including

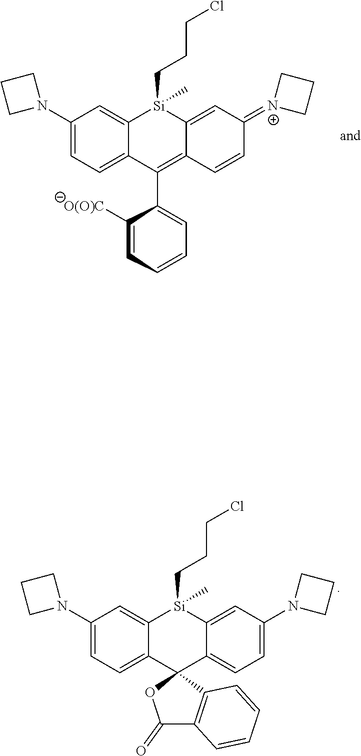

“Ring-closed tautomer,” used herein, refers to the spirocyclic tautomer of a compound of the present disclosure characterized by a covalent bond between the carbon atom of C-Q and a heteroatom (e.g., O, N) of a nucleophilic substituent of Q or a nucleophilic value of R⁴ or R⁵ (e.g., —P(O)OH(OR⁵⁰), —O—P(O)OH(OR⁵¹), SO₃H, —C(O)NH(R⁴⁰), (C₁-C₆)alkyl-OH, —OH, or —C(O)OH). The various spirocyclic tautomers of a compound of the present disclosure include, but are not limited to, spirolactones, spirolactams, spirocyclic ethers, spirocyclic thioethers, spirosultones, and spirophostones. In some embodiments, a tautomer is a ring-closed tautomer of a reference compound (e.g., a compound of the present disclosure). Examples of ring-closed tautomers are shown below:

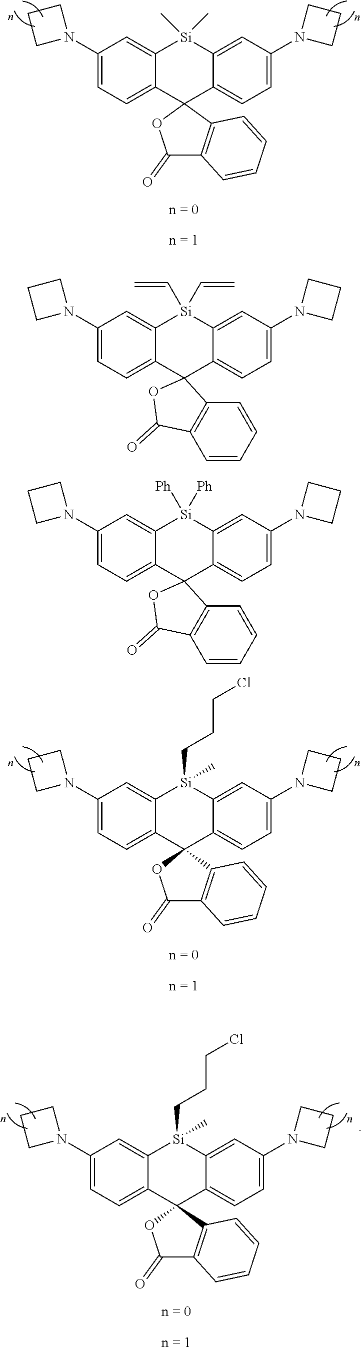

Any formula given herein is intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶Cl, ¹²³J, ¹²⁴I and ¹²⁵I, respectively. The present disclosure includes various isotopically labeled compounds as defined herein, for example those into which radioactive isotopes, such as ³H and ₁₄C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Isotopically labeled compounds of the present disclosure can generally be prepared by conventional techniques known to those skilled in the art or by processes disclosed in the schemes or in the examples and preparations described below (or analogous processes to those described hereinbelow), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Examples of acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art such as ion-exchange. Other acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In some embodiments, exemplary inorganic acids which form suitable salts include, but are not limited to, hydrochloric, hydrobromic, sulfuric and phosphoric acid and acid metal salts, such as sodium monohydrogen orthophosphate and potassium hydrogen sulfate. Illustrative organic acids which form suitable salts include mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids, such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms.

In some embodiments, acid addition salts are most suitably formed from acids, and include, for example, those formed with inorganic acids, e.g., hydrochloric, sulfuric or phosphoric acids, and organic acids, e.g., succinic, maleic, acetic or fumaric acid.

Illustrative inorganic bases which form suitable salts include, but are not limited to, lithium, sodium, potassium, calcium, magnesium or barium hydroxides. Illustrative organic bases which form suitable salts include aliphatic, alicyclic or aromatic organic amines, such as methylamine, trimethyl amine and picoline, or ammonia. The selection criteria for the appropriate salt will be known to one skilled in the art.

Salts derived from appropriate bases include alkali metal, alkaline earth metal, ammonium and N⁺((C₁-C₄) alkyl)₄ salts. Representative alkali or alkaline earth metal salts include sodium, lithium, potassium, calcium, magnesium, and the like. Further salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

A description of example embodiments follows.

### Compounds

A first embodiment is a compound having the following structural formula:

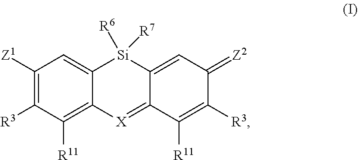

or a tautomer thereof, or a salt of the foregoing, wherein:
   X is C-Q or N, and when X is C-Q, each R¹¹ is H, and when X is N, each R¹¹ is independently H, (C₁-C₆)alkyl or halo;
   Z¹ and Z² are —N(R¹)(R²); and
   R¹ is —H, (C₁-C₆)alkyl, (C₆-C₁₅)aryl or (C₅-C₁₅)heteroaryl; R² is —H, (C₁-C₆)alkyl, (C₆-C₁₅)aryl or (C₅-C₁₅)heteroaryl; and R³ is —H, fluoro, or choro; or
   R¹ is —H, (C₁-C₆)alkyl, (C₆-C₁₅)aryl or (C₅-C₁₅)heteroaryl; and R² and R³, taken together with their intervening atoms, form a (C₄-C₈)heterocyclyl (in some aspects, a (C₅-C₈)heterocyclyl); or
   R¹ and R², taken together with the N atom to which they are attached, form a (C₃-C₈)heterocyclyl; and R³ is —H, fluoro, or chloro; or
   Z¹ is OH and Z² is O; and R³ is —H, fluoro, or chloro; Q is Ar, (C₁-C₆)alkyl, (C₁-C₆)alkenyl, (C₁-C₆)alkynyl, (C₁-C₆)alkyl-Ar, (C₁-C₆)alkenyl-Ar, (C₁-C₆)alkynyl-Ar, or (C₅-C₁₂)cycloalkenyl-Ar, wherein the alkyl, alkenyl, or alkynyl is optionally substituted with one or more groups independently selected from —P(O)OH(OR⁵⁰), —O—P(O)OH(OR⁵¹), —SO₃H, —C(O)NH(R⁴⁰), (C₁-C₆)alkyl-OH, —OH, or —C(O)OH;
   Ar is a ring having the structure:

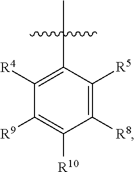

wherein:
R⁴ and R⁵ are each independently —H, —CO₂H, halo, cyano, —OH, (C₁-C₆)alky-OH, —SO₃H, nitro, triflate, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl, —C(O)NH(R⁴⁰), —P(O)(OR⁵⁰)₂, —O—P(O)(OR⁵¹)₂, or (C₃-C₈)cyclic amino;
each R⁴⁰ is independently —H, cyano, or SO₂(R⁴¹);
each R⁴¹ is independently (C₁-C₆)alkyl, NH₂, NH((C₁-C₆)alkyl), or N((C₁-C₆)alkyl)₂; each R⁵⁰ is independently-H, (C₁-C₆)alkyl, or acetoxymethyl;
each R⁵¹ is independently —H, (C₁-C₆)alkyl, or acetoxymethyl;
R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R⁸⁰;
   each R⁸⁰ is independently selected from oxo, halo, —CO₂H, —C(O)O—N-succinimide, maleimido, azido, propargyl, norbornenyl, or tetrazinyl, or a sensor or targeting group (in some aspects, oxo, halo, —CO₂H, —C(O)O—N-succinimide, maleimido, azido, or tetrazinyl, or a sensor or targeting group);
R⁹ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; and
R¹⁰ —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R¹⁰⁰;
   each R¹⁰⁰ is independently selected from oxo, halo, —CO₂H, —C(O)O—N-succinimide, maleimido, azido, propargyl, norbornenyl or tetrazinyl, or a sensor or targeting group (in some aspects, oxo, halo, —CO₂H, —C(O)O—N-succinimide, maleimido, azido, or tetrazinyl, or a sensor or targeting group); or
Ar is a ring having the structure:

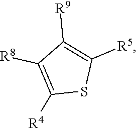

wherein one of R⁴, R⁵, R⁸, and R⁹ is covalently attached to X, and the rest of R⁴, R⁵, R⁸, and R⁹ are as defined above; and
R⁶ is (C₂-C₂₅)aliphatic, (C₂-C₂₅)heteroaliphatic, (C₆-C₁₅)aryl or (C₅-C₁₅)heteroaryl; and
R⁷ is (C₁-C₂₅)aliphatic, (C₂-C₂₅)heteroaliphatic, (C₆-C₁₅)aryl or (C₅-C₁₁)heteroaryl; or R⁶ and R⁷, taken together with the Si atom to which they are attached, form a (C₅-C₈)silacycle, wherein:
   the aliphatic and heteroaliphatic of R⁶ and R⁷, or the silacycle formed by R⁶ and R⁷, taken together with the Si atom to which they are attached, are optionally substituted with one or more R⁶⁰, and the aryl or heteroaryl of R⁶ and R⁷ are optionally substituted with one or more R⁶¹;
   each R⁶⁰ is independently selected from oxo, halo, —CO₂H, —C(O)O—N-succinimide, maleimido, amino, hydroxyl, thiol, azido, propargyl, norbornenyl or tetrazinyl, or a sensor or targeting group (in some aspects, oxo, halo, —CO₂H, —C(O)O—N-succinimide, maleimido, amino, hydroxyl, thiol, azido, or tetrazinyl, or a sensor or targeting group);
each R⁶¹ is independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H.

In a first aspect of the first embodiment:
Z¹ and Z² are —N(R¹)(R²); and
R¹ is —H, (C₁-C₆)alkyl, (C₆-C₁₅)aryl or (C₅-C₁₁)heteroaryl; R² is —H, (C₁-C₆)alkyl, (C₆-C₁₅)aryl or (C₅-C₁₅)heteroaryl; and R³ is —H, fluoro, or choro; or
R¹ is —H, (C₁-C₆)alkyl, (C₆-C₁₅)aryl or (C₅-C₁₅)heteroaryl; and R² and R³, taken together with their intervening atoms, form a (C₅-C₈)heterocyclyl; or
R¹ and R², taken together with the N atom to which they are attached, form a (C₃-C₈)heterocyclyl; and R³ is —H, fluoro, or chloro; or
Z¹ is OH and Z² is O; and R³ is —H, fluoro, or chloro;
Ar is a ring having the structure:

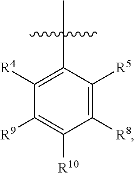

wherein:
R⁴ and R⁵ are each independently —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R⁸⁰;
   each R⁸⁰ is independently selected from oxo, halo, —CO₂H, —C(O)O—N-succinimide, maleimido, azido or tetrazinyl, or a sensor or targeting group;
R⁹ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; and
R¹⁰ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; or
Ar is a ring having the structure:

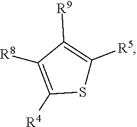

wherein one of R⁴, R⁵, R⁸, and R⁹ is covalently attached to X, and the rest of R⁴, R⁵, R⁸, and R⁹ are as defined above; and
R⁶ is (C₂-C₂₅)aliphatic, (C₂-C₂₅)heteroaliphatic, (C₆-C₁₅)aryl or (C₅-C₁₅)heteroaryl; and
R⁷ is (C₁-C₂₅)aliphatic, (C₂-C₂₅)heteroaliphatic, (C₆-C₁₅)aryl or (C₅-C₁₅)heteroaryl; or R⁶ and R⁷, taken together with the Si atom to which they are attached, form a (C₅-C₈)silacycle, wherein:
   the aliphatic and heteroaliphatic of R⁶ and R⁷, or the silacycle formed by R⁶ and R⁷, taken together with the Si atom to which they are attached, are optionally substituted with one or more R⁶⁰, and the aryl or heteroaryl of R⁶ and R⁷ are optionally substituted with one or more R⁶¹;
   each R⁶⁰ is independently selected from oxo, halo, —CO₂H, —C(O)O—N-succinimide, maleimido, amino, hydroxyl, thiol, azido or tetrazinyl, or a sensor or targeting group;
   each R⁶¹ is independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H.

In a second aspect of the first embodiment, R¹ is (C₁-C₆)alkyl; R² is (C₁-C₆)alkyl; and R³ is H, fluoro or chloro. Values for the remaining variables are as described in the first embodiment, or first aspect thereof.

In a third aspect of the first embodiment, wherein R¹ is methyl or ethyl; R² is methyl or ethyl; and R³ is H. Values for the remaining variables are as described in the first embodiment, or first or second aspect thereof.

In a fourth aspect of the first embodiment, R¹ is (C₁-C₆)alkyl; and R² and R³, taken together with their intervening atoms, form a (C₅-C₈)heterocyclyl. Values for the remaining variables are as described in the first embodiment, or first through third aspect thereof.

In a fifth aspect of the first embodiment, R¹ is methyl; and R² and R³, taken together with their intervening atoms, form a (C₅-C₆)cyclic amino. Values for the remaining variables are as described in the first embodiment, or first through fourth aspects thereof.

In a sixth aspect of the first embodiment, R¹ and R², taken together with the N atom to which they are attached, form aziridinyl or azetidinyl (in some aspects, azetidinyl); and R³ is —H, fluoro or chloro. Values for the remaining variables are as described in the first embodiment, or first through fifth aspects thereof.

In a seventh aspect of the first embodiment, R⁴ is —H, —CO₂H, (C₁-C₆)alkyl, (C₁-C₆)haloalkyl, (C₁-C₆)alkoxy or (C₁-C₆)haloalkoxy. Values for the remaining variables are as described in the first embodiment, or first through sixth aspects thereof.

In an eighth aspect of the first embodiment, R⁴ is —H, —CO₂H, methyl or methoxy. Values for the remaining variables are as described in the first embodiment, or first through seventh aspects thereof.

In a ninth aspect of the first embodiment, R⁵ is —H, (C₁-C₆)alkyl, (C₁-C₆)haloalkyl, (C₁-C₆)alkoxy or (C₁-C₆)haloalkoxy. Values for the remaining variables are as described in the first embodiment, or first through eighth aspects thereof.

In a tenth aspect of the first embodiment, R⁵ is —H, methyl or methoxy. Values for the remaining variables are as described in the first embodiment, or first through ninth aspects thereof.

In an eleventh aspect of the first embodiment, R⁴ and R⁵ are the same. Values for the variables (including R⁴ and R⁵) are as described in the first embodiment, or first through tenth aspects thereof.

In a twelfth aspect of the first embodiment, R⁴ and R⁵ are different from one another. Values for the variables (including R⁴ and R⁵) are as described in the first embodiment, or first through tenth aspects thereof.

In a thirteenth aspect of the first embodiment, R⁴ is —CO₂H and R⁵ is —H. Values for the remaining variables are as described in the first embodiment, or first through twelfth aspects thereof.

In a fourteenth aspect of the first embodiment, R⁶ is optionally substituted (C₂-C₁₅)aliphatic, (C₂-C₁₅)heteroaliphatic, (C₆-C₁₅)aryl or (C₅-C₁₅)heteroaryl; and R⁷ is optionally substituted (C₁-C₁₅)aliphatic, (C₂-C₁₅)heteroaliphatic, (C₆-C₁₅)aryl or (C₅-C₁₅)heteroaryl. Values for the remaining variables are as described in the first embodiment, or first through thirteenth aspects thereof.

In a fifteenth aspect of the first embodiment, R⁶ is optionally substituted (C₂-C₁₅)aliphatic. Values for the remaining variables are as described in the first embodiment, or first through fourteenth aspects thereof.

In a sixteenth aspect of the first embodiment, R⁶ is optionally substituted (C₂-C₁₅)alkyl, (C₂-C₁₅)alkenyl, (C₂-C₁₅)alkynyl, (C₃-C₁₅)cycloalkenyl or (C₅-C₁₅)cycloalkynyl. Values for the remaining variables are as described in the first embodiment, or first through fifteenth aspects thereof.

In a seventeenth aspect of the first embodiment, R⁶ is optionally substituted (C₂-C₁₅)heteroaliphatic. Values for the remaining variables are as described in the first embodiment, or first through sixteenth aspects thereof.

In an eighteenth aspect of the first embodiment, R⁶ is optionally substituted (C₆-C₁₅)aryl or (C₅-C₁₅)heteroaryl. Values for the remaining variables are as described in the first embodiment, or first through seventeenth aspects thereof.

In a nineteenth aspect of the first embodiment, R⁶ is optionally substituted ethyl, propyl, vinyl, phenyl, octyl, octadecyl or norbornenyl. Values for the remaining variables are as described in the first embodiment, or first through eighteenth aspects thereof.

In a twentieth aspect of the first embodiment, R⁷ is optionally substituted (C₁-C₁₅)aliphatic. Values for the remaining variables are as described in the first embodiment, or first through nineteenth aspects thereof.

In a twenty-first aspect of the first embodiment, R⁷ is optionally substituted (C₂-C₁₅)heteroaliphatic. Values for the remaining variables are as described in the first embodiment, or first through twentieth aspects thereof.

In a twenty-second aspect of the first embodiment, R⁷ is optionally substituted (C₆-C₁₅)aryl or (C₅-C₁₅)heteroaryl. Values for the remaining variables are as described in the first embodiment, or first through twenty-first aspects thereof.

In a twenty-third aspect of the first embodiment, R⁷ is optionally substituted methyl, ethyl, phenyl, vinyl, octyl or octadecyl. Values for the remaining variables are as described in the first embodiment, or first through twenty-second aspects thereof.

In a twenty-fourth aspect of the first embodiment, R⁷ is methyl. Values for the remaining variables are as described in the first embodiment, or first through twenty-third aspects thereof.

In a twenty-fifth aspect of the first embodiment, R⁶ and R⁷ are the same. Values for the variables (including R⁶ and R⁷) are as described in the first embodiment, or first through twenty-fourth aspects thereof.

In a twenty-sixth aspect of the first embodiment, R⁶ and R⁷ are different from one another. Values for the variables (including R⁶ and R⁷) are as described in the first embodiment, or first through twenty-fourth aspects thereof.

In a twenty-seventh aspect of the first embodiment, R⁶ and R⁷, taken together with the Si atom to which they are attached, form an optionally substituted (C₅-C₈)silacycloalkyl. Values for the remaining variables are as described in the first embodiment, or first through twenty-sixth aspects thereof.

In a twenty-eighth aspect of the first embodiment, each R⁶⁰ is independently selected from oxo, halo, —CO₂H, —C(O)O—N-succinimide, maleimido, amino, hydroxyl, thiol, azido or tetrazinyl. Values for the remaining variables are as described in the first embodiment, or first through twenty-seventh aspects thereof.

In a twenty-ninth aspect of the first embodiment, each R⁶⁰ is independently selected from oxo, or a sensor or targeting group. Values for the remaining variables are as described in the first embodiment, or first through twenty-eighth aspects thereof.

In a thirtieth aspect of the first embodiment, each R⁶¹ is independently selected from halo or (C₁-C₆)dialkylamino. Values for the remaining variables are as described in the first embodiment, or first through twenty-ninth aspects thereof.

In a thirty-first aspect of the first embodiment, R⁸ is —H or carboxy. Values for the remaining variables are as described in the first embodiment, or first through thirtieth aspects thereof.

In a thirty-second aspect of the first embodiment, R⁸ is —H. Values for the remaining variables are as described in the first embodiment, or first through thirty-first aspects thereof.

In a thirty-third aspect of the first embodiment, R⁸ is optionally substituted (C₁-C₂₅)aliphatic or (C₂-C₂₅)heteroaliphatic. Values for the remaining variables are as described in the first embodiment, or first through thirty-second aspects thereof.

In a thirty-fourth aspect of the first embodiment, each R⁸⁰ is independently oxo, halo, —CO₂H, —C(O)O—N-succinimide, maleimido, azido, propargyl, norbornenyl, or tetrazinyl (in some aspects, oxo, halo, —CO₂H, —C(O)O—N-succinimide, maleimido, azido, or tetrazinyl). Values for the remaining variables are as described in the first embodiment, or first through thirty-third aspects thereof.

In a thirty-fifth aspect of the first embodiment, each R⁸⁰ is independently oxo or a sensor or targeting group. Values for the remaining variables are as described in the first embodiment, or first through thirty-fourth aspects thereof.

In a thirty-sixth aspect of the first embodiment, R⁹ is —H. Values for the remaining variables are as described in the first embodiment, or first through thirty-fifth aspects thereof.

In a thirty-seventh aspect of the first embodiment, R¹⁰ is —H. Values for the remaining variables are as described in the first embodiment, or first through thirty-sixth aspects thereof.

In a thirty-eighth aspect of the first embodiment, X is C-Q. Values for the remaining variables are as described in the first embodiment, or first through thirty-seventh aspects thereof.

In a thirty-ninth aspect of the first embodiment, X is N. Values for the remaining variables are as described in the first embodiment, or first through thirty-eighth aspects thereof.

In a fortieth aspect of the first embodiment, Ar is

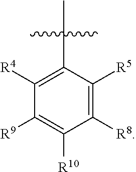

Values for the remaining variables (including R⁴, R⁵, R¹, R⁹, R¹⁰) are as described in the first embodiment, or first through thirty-ninth aspects thereof.

In a forty-first aspect of the first embodiment, Ar is

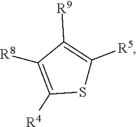

Values for the remaining variables (including R⁴, R⁵, R⁸, R⁹) are as described in the first embodiment, or first through fortieth aspects thereof.

In a forty-second aspect of the first embodiment, when X is N, each R¹¹ is H, methyl, or fluorine. Values for the remaining variables are as described in the first embodiment, or first through forty-first aspects thereof.

In a forty-third aspect of the first embodiment, when X is N, each R¹¹ is H, (C₁-C₆)alkyl or halo. Values for the remaining variables are as described in the first embodiment, or first through forty-second aspects thereof.

In a forty-fourth aspect of the first embodiment, R⁶ is optionally substituted (C₂-C₁₅)aliphatic or (C₂-C₁₅)heteroaliphatic. Values for the remaining variables are as described in the first embodiment, or first through forty-third aspects thereof.

In a forty-fifth aspect of the first embodiment, R⁶ is ethyl, vinyl,

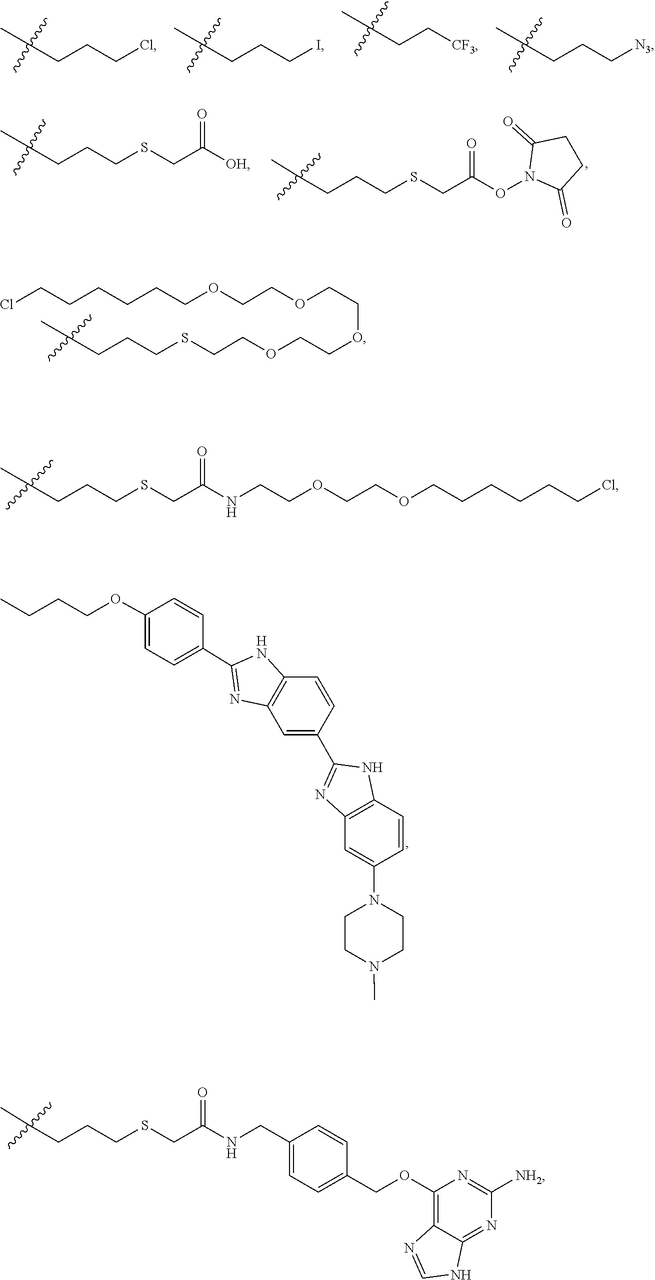

octyl, octadecyl, norbornenyl, phenyl, or dimethylaminophenyl. Values for the remaining variables are as described in the first embodiment, or first through forty-fourth aspects thereof.

In a forty-sixth aspect of the first embodiment, R⁷ is methyl, ethyl, phenyl, vinyl, octyl or octadecyl. Values for the remaining variables are as described in the first embodiment, or first through forty-fifth aspects thereof.

In a forty-seventh aspect of the first embodiment, R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino. Values for the remaining variables are as described in the first embodiment, or first through forty-sixth aspects thereof.

In a forty-eighth aspect of the first embodiment, R¹⁰ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino. Values for the remaining variables are as described in the first embodiment, or first through forty-seventh aspects thereof.

A second embodiment is a compound of Formula (Ia):

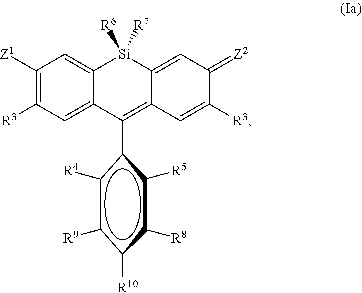

or a tautomer thereof, or a salt of the foregoing. Values for the variables (e.g., Z¹, Z², R³, R⁴, R⁵, R⁶, R⁷, R⁸, R⁹, R¹⁰) are as described in the first embodiment, or any aspect thereof.

A third embodiment is a compound of Formula (Ib):

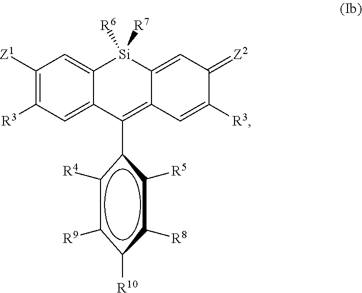

or a tautomer thereof, or a salt of the foregoing. Values for the variables (e.g., Z¹, Z², R³, R⁴, R⁵, R⁶, R⁷, R⁸, R⁹, R¹⁰) are as described in the first embodiment, or any aspect thereof.

A fourth embodiment is a compound of Formula (II):

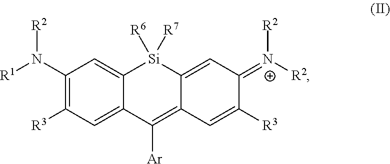

or a tautomer thereof, or a salt of the foregoing. Values for the variables (e.g., Ar, R¹, R², R³, R⁶, R⁷) are as described in the first embodiment, or any aspect thereof.

A fifth embodiment is a compound of Formula (IIa):

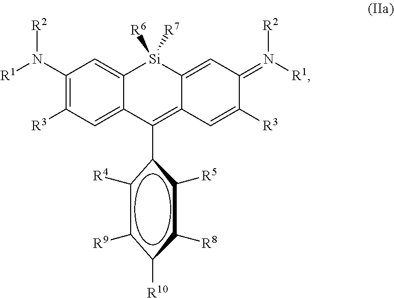

or a tautomer thereof, or a salt of the foregoing. Values for the variables (e.g., R¹, R², R³, R⁴, R⁵, R⁶, R⁷, R⁸, R⁹, R¹⁰) are as described in the first embodiment, or any aspect thereof.

A sixth embodiment is a compound of Formula (IIb):

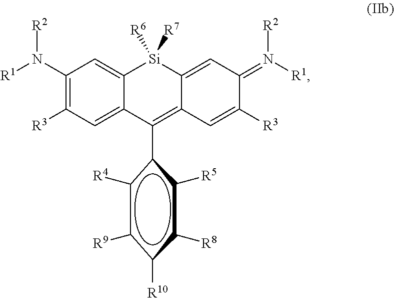

or a tautomer thereof, or a salt of the foregoing. Values for the variables (e.g., R¹, R², R³, R⁴, R⁵, R⁶, R⁷, R⁸, R⁹, R¹⁰) are as described in the first embodiment, or any aspect thereof.

A seventh embodiment is a compound of Formula (III):

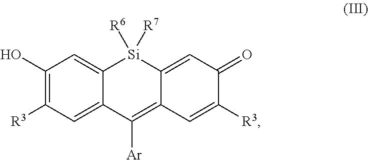

or a tautomer thereof, or a salt of the foregoing. Values for the variables (e.g., Ar, R³, R⁶, R⁷) are as described in the first embodiment, or any aspect thereof.

An eighth embodiment is a compound of Formula (IV):

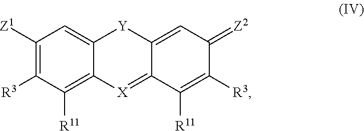

or a tautomer thereof, or a salt of the foregoing, wherein Y is Si(R⁶)(R⁷), Ge(R⁶)(R⁷), or P(O)R⁶. Values for the remaining variables (e.g., X, R³, R⁶, R⁷, Z¹, Z², R¹¹) are as described in the first embodiment, or any aspect thereof.

In a first aspect of the eighth embodiment, when Y is P(O)R⁶, R⁶ is not phenyl, methyl, or ethoxy. Values for the remaining variables are as described in the first embodiment, or any aspect thereof, or eighth embodiment.

A ninth embodiment is a compound of any one of Formulas I-IV, or a tautomer thereof, or a salt of the foregoing, wherein R⁶ is (C₁-C₂₅)aliphatic or (C₂-C₂₅)heteroaliphatic substituted with a leaving group. Values for the remaining variables are as described in the first embodiment, or any aspect thereof.

A tenth embodiment is a compound of any one of Formulas I-IV, or a tautomer thereof, or a salt of the foregoing, wherein R⁶ is (C₁-C₂₅)aliphatic, (C₂-C₂₅)heteroaliphatic, (C₆-C₁₅)aryl or (C₅-C₁₅)heteroaryl, provided that R⁶ and R⁷ are not both CH₃. Values for the remaining variables are as described in the first embodiment, or any aspect thereof.

Representative examples of compounds of the present disclosure are depicted in Table 1. One embodiment is a compound of a structural formula depicted in Table 1, or a tautomer thereof (e.g., ring-closed tautomer thereof), or a salt of the foregoing.

TABLE 1
Representative compounds of the present disclosure.

| Compound No. | Compound Structure |
|---|---|
| 002 | 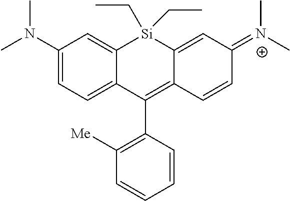 |
| 003 | 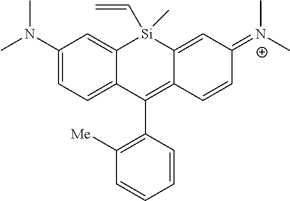 |
| 004 | 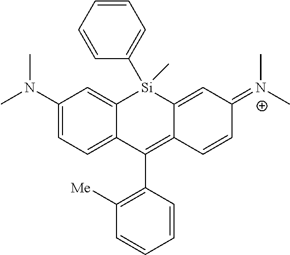 |
| 005 | 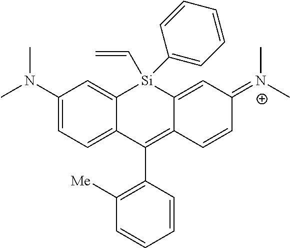 |
| 006 | 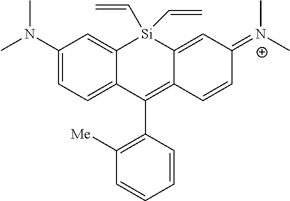 |
| 007 | 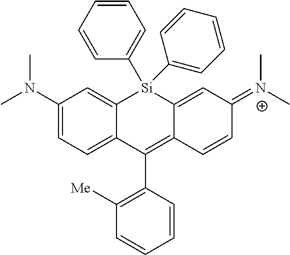 |
| 008 | 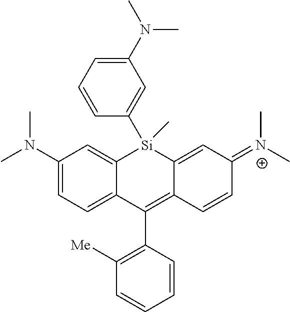 |
| 009 | 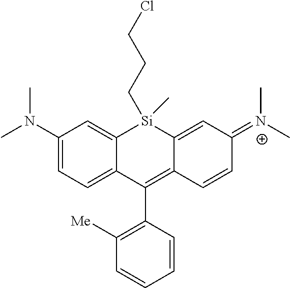 |
| 010 | 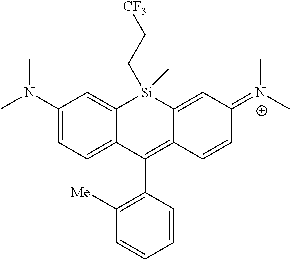 |
| 011 | 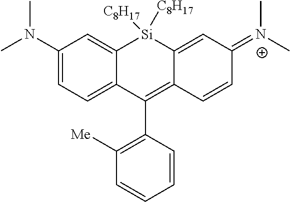 |
| 012 | 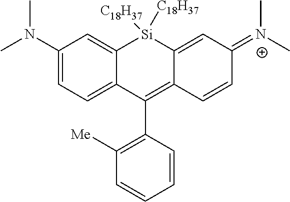 |
| 013 | 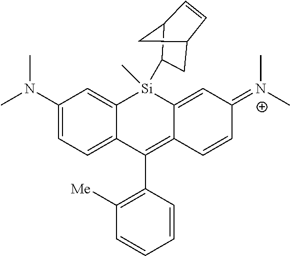 |
| 014 | 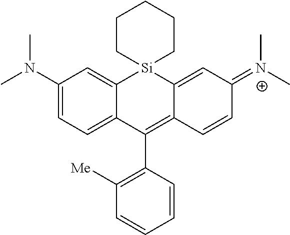 |
| 015 | 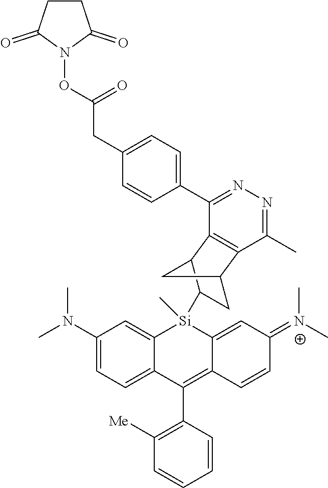 [SMILES: Cc1ccccc1C1=C2C=CC(=[N+](C)C)C=C2[Si](C)(C2CC3CC2c2c(-c4ccc(CC(=O)ON5C(=O)CCC5=O)cc4)nnc(C)c23)c2cc(N(C)C)ccc21] |
| 017 | 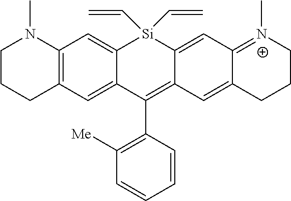 [SMILES: C=C[Si]1(C=C)c2cc3c(cc2C(c2ccccc2C)=c2cc4c(cc21)=[N+](C)CCC4)CCCN3C] |
| 018 | 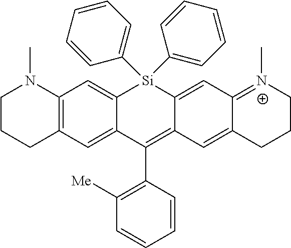 [SMILES: Cc1ccccc1C1=c2cc3c(cc2[Si](c2ccccc2)(c2ccccc2)c2cc4c(cc21)CCCN4C)=[N+](C)CCC3] |
| 019 | 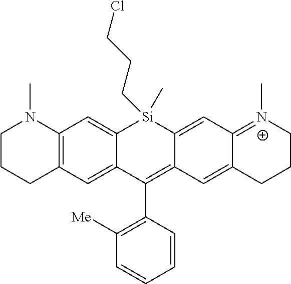 [SMILES: Cc1ccccc1C1=c2cc3c(cc2[Si](C)(CCCCl)c2cc4c(cc21)CCCN4C)=[N+](C)CCC3] |
| 021 | 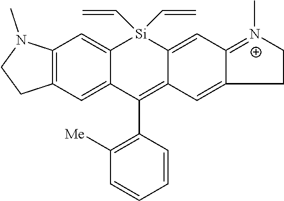 [SMILES: C=C[Si]1(C=C)c2cc3c(cc2C(c2ccccc2C)=c2cc4c(cc21)=[N+](C)CC4)CCN3C] |
| 022 | 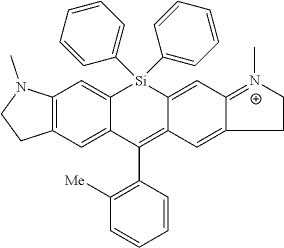 [SMILES: Cc1ccccc1C1=c2cc3c(cc2[Si](c2ccccc2)(c2ccccc2)c2cc4c(cc21)CCN4C)=[N+](C)CC3] |
| 023 | 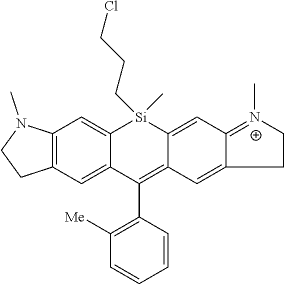 [SMILES: Cc1ccccc1C1=c2cc3c(cc2[Si](C)(CCCCl)c2cc4c(cc21)CCN4C)=[N+](C)CC3] |
| 025 | 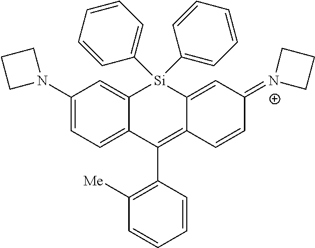 [SMILES: Cc1ccccc1C1=C2C=CC(=[N+]3CCC3)C=C2[Si](c2ccccc2)(c2ccccc2)c2cc(N3CCC3)ccc21] |
| 026 | 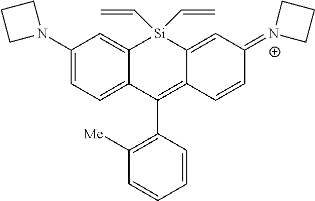 [SMILES: C=C[Si]1(C=C)C2=CC(=[N+]3CCC3)C=CC2=C(c2ccccc2C)c2ccc(N3CCC3)cc21] |
| 027 | 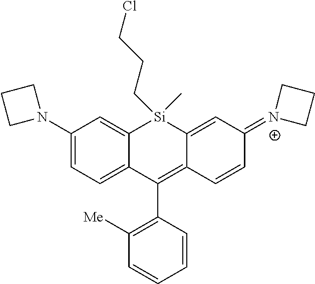 [SMILES: Cc1ccccc1C1=C2C=CC(=[N+]3CCC3)C=C2[Si](C)(CCCCl)c2cc(N3CCC3)ccc21] |
| 028 | 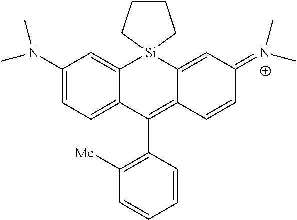 |
| 029 | 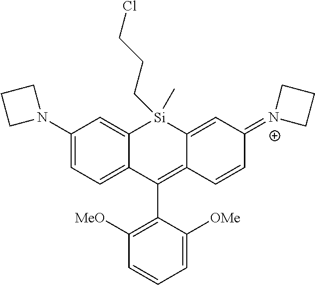 [SMILES: COc1cccc(OC)c1C1=C2C=CC(=[N+]3CCC3)C=C2[Si](C)(CCCCl)c2cc(N3CCC3)ccc21] |
| 030 | 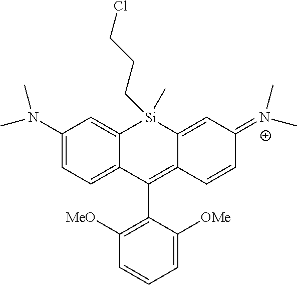 |
| 033-O | 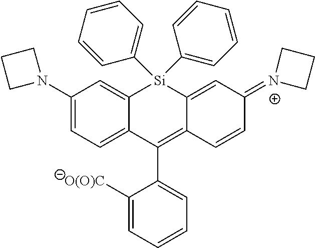 |
| 034-O | 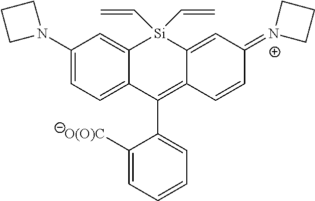 |
| 035-O | 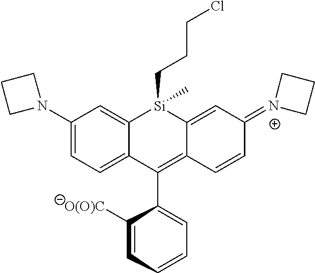 |
| 036-O | 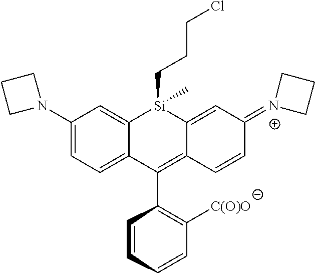 |
| 037-O | 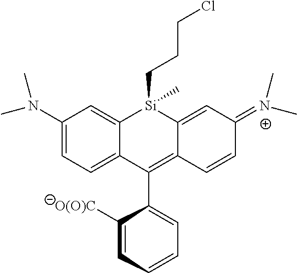 |
| 038-O | 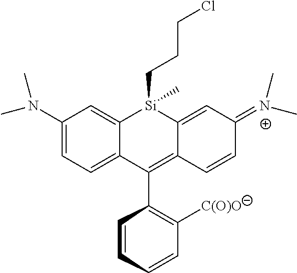 |
| 039-O | 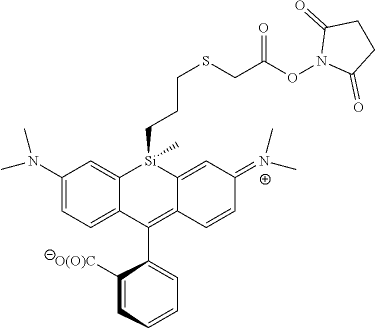 |
| 040-O | 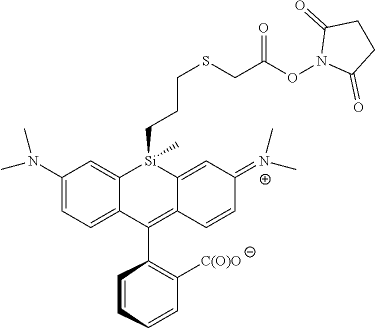 |
| 041 | 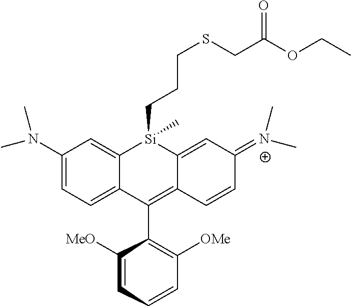 |
| 042-O | 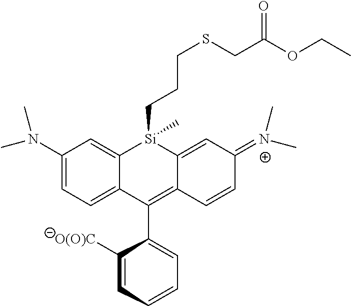 |
| 043-O | 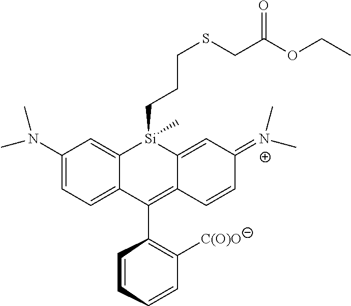 |
| 044-O | 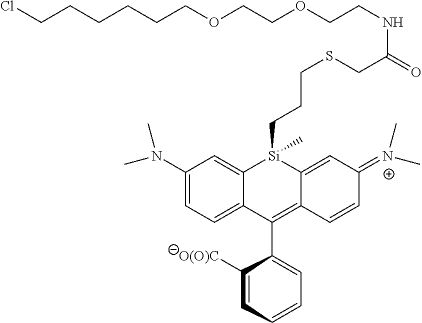 |
| 045-O | 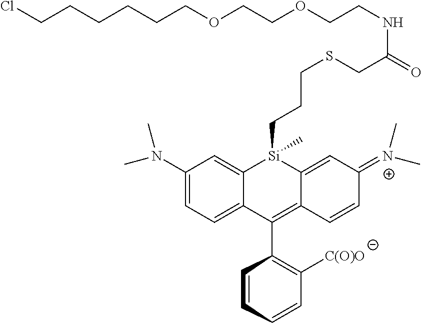 |
| 046 | 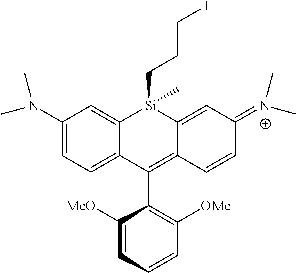 |
| 047-O | 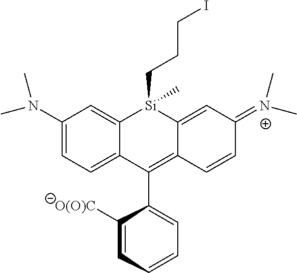 |
| 048-O | 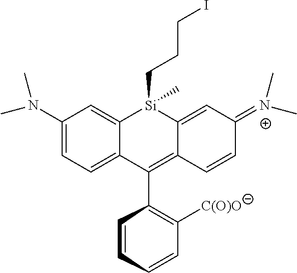 |
| 049 | 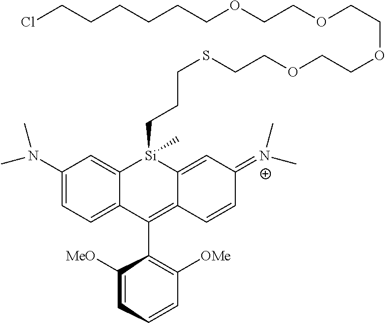 |
| 050-O | 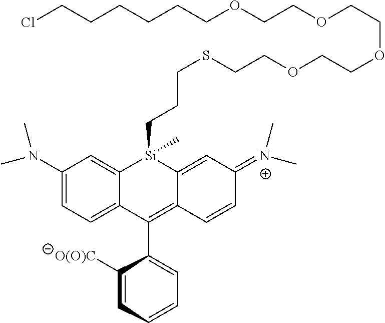 |
| 051-O | 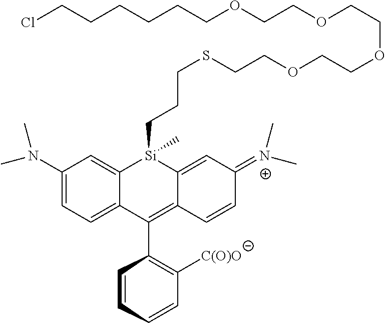 |
| 052 | 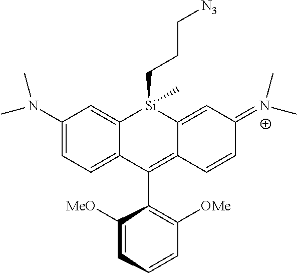 |
| 053-O | 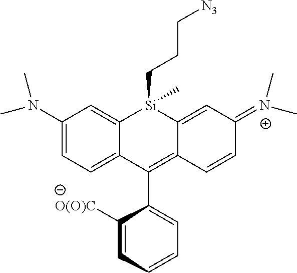 |
| 054-O | 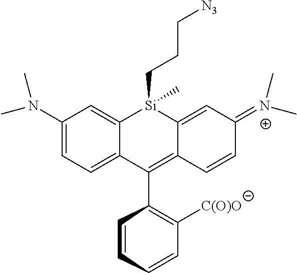 |
| 055 | 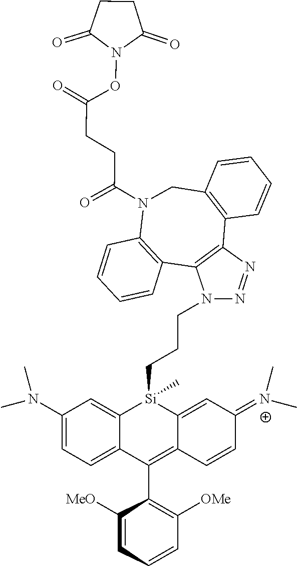 |
| 056-O | 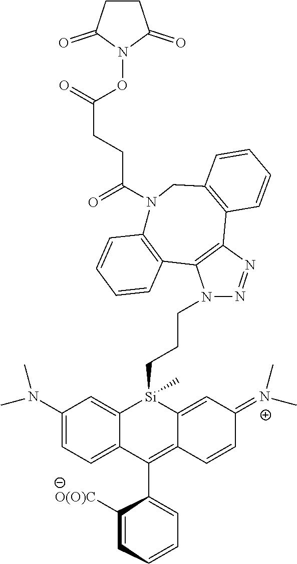 |
| 057-O | 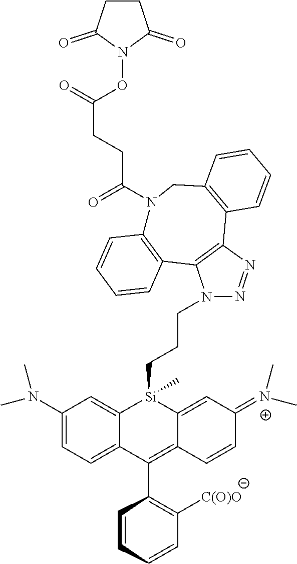 |
| 058 | 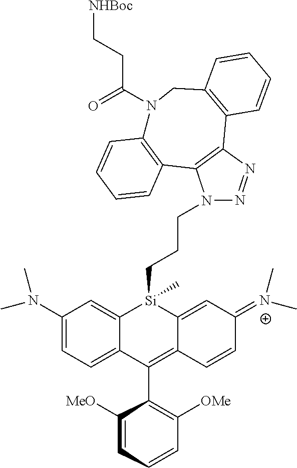 |
| 059-O | 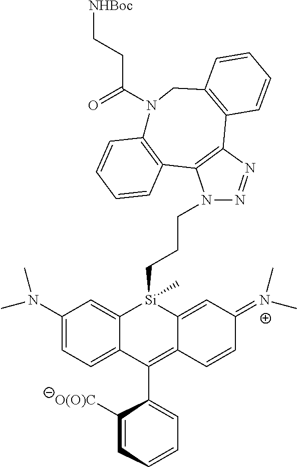 |
| 060-O | 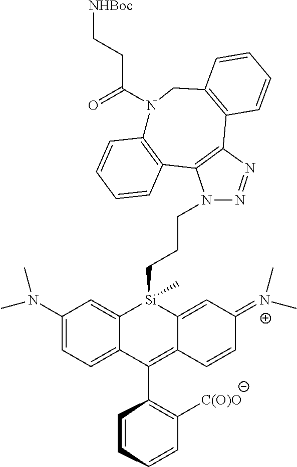 |
| 061-O | 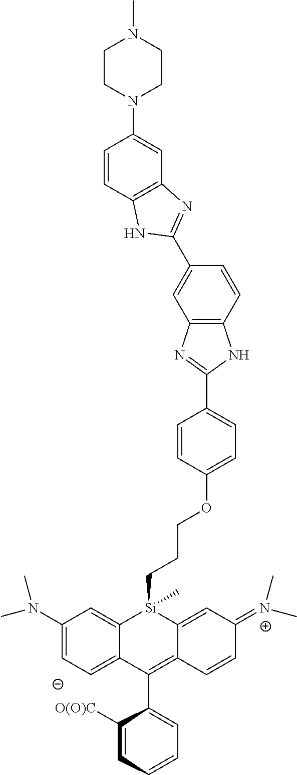 |
| 062-O | 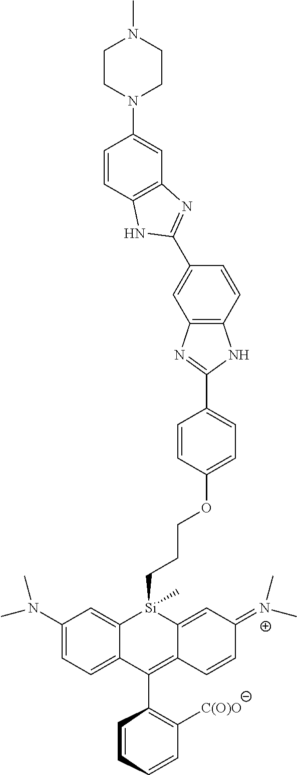 |
| 063-O | 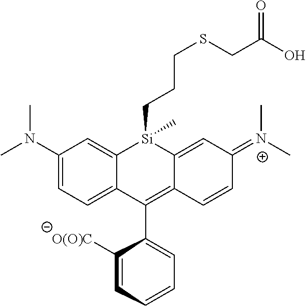 |
| 064-O | 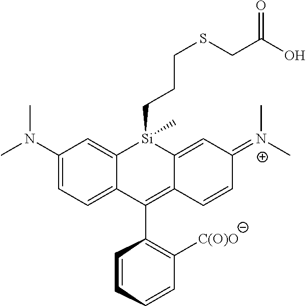 |
| 065-O | 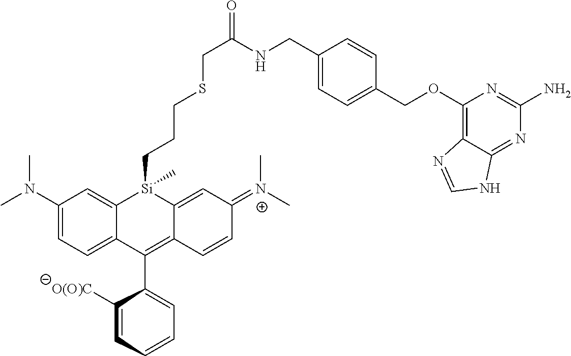 |
| 066-O | 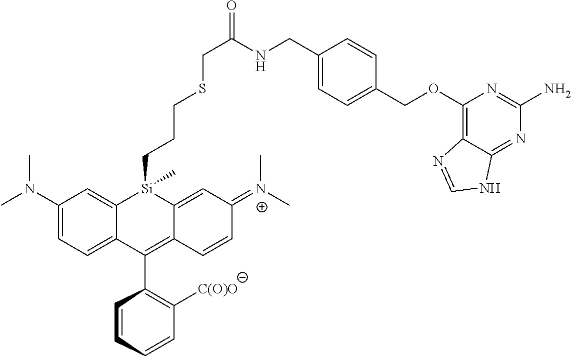 |
| 067 | 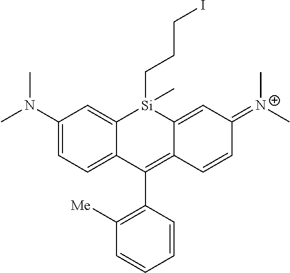 |
| 068-O | 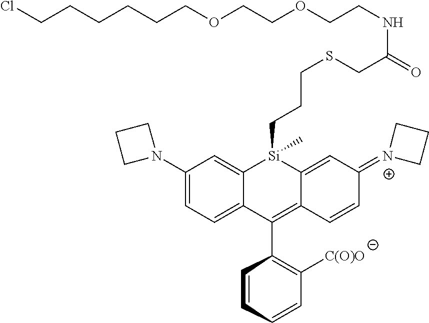 |
| 069-O | 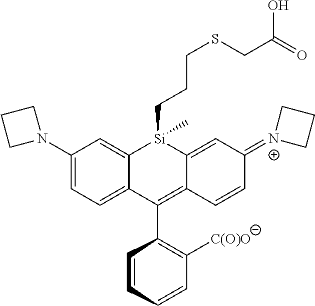 |
| 070-O | 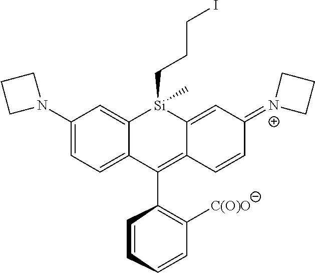 |

Other specific examples of values for the variables of any one of Formulas I-IV described herein, in particular X and Q, can be found, for example, in Wang et al. (A general strategy to develop cell permeable and fluorogenic probes for multicolour nanoscopy. Nat. Chem. 12, 165-172 (2020)) and Butkevich (Modular Synthetic Approach to Silicon-Rhodamine Homologues and Analogues via Bis-aryllanthanum Reagents. Organic Letters 2021 23 (7), 2604-2609), the entire contents of which are incorporated herein by reference.

### Methods

The compounds described herein can be synthesized and modified using methods set forth herein, as well as techniques known in the art. Substituents and combinations of substituents in the methods described herein are preferably those that are not only chemically stable, but also chemically compatible with the conditions to which the compound is being subjected and/or the desired modification and/or use.

Another embodiment is a method of modifying a compound of the present disclosure comprising a leaving group, e.g., a compound of any one of Formulas I-IV, or a tautomer thereof, or a salt of the foregoing, wherein R⁶ is (C₁-C₂₅)aliphatic or (C₂-C₂₅)heteroaliphatic substituted with a leaving group. The method comprises reacting the compound of the present disclosure, or a tautomer thereof, or a salt of the foregoing, or an appropriately protected derivative of any of the foregoing, with a nucleophile under conditions suitable for the nucleophile to displace the leaving group, thereby modifying the compound. In some aspects, the compound of the present disclosure has the following structural formula:

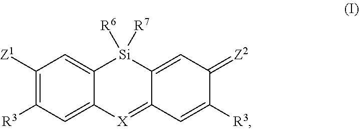

or a tautomer thereof, or a salt of the foregoing, wherein:
   X is C-Q or N, and when X is C-Q, each R¹¹ is H, and when X is N, each R¹¹ is independently H, (C₁-C₆)alkyl or halo;
   Z¹ and Z² are —N(R¹)(R²); and
   R¹ is —H, (C₁-C₆)alkyl, (C₆-C₁₅)aryl or (C₅-C₁₅)heteroaryl; R² is —H, (C₁-C₆)alkyl, (C₆-C₁₅)aryl or (C₅-C₁₅)heteroaryl; and R³ is —H, fluoro, or choro; or
   R¹ is —H, (C₁-C₆)alkyl, (C₆-C₁₅)aryl or (C₅-C₁₅)heteroaryl; and R² and R³, taken together with their intervening atoms, form a (C₄-C₈)heterocyclyl; or
   R¹ and R², taken together with the N atom to which they are attached, form a (C₃-C₈)heterocyclyl; and R³ is —H, fluoro, or chloro; or
   Z¹ is OH and Z² is O; and R³ is —H, fluoro, or chloro;
   Q is Ar, (C₁-C₆)alkyl, (C₁-C₆)alkenyl, (C₁-C₆)alkynyl, (C₁-C₆)alkyl-Ar, (C₁-C₆)alkenyl-Ar, (C₁-C₆)alkynyl-Ar, or (C₅-C₁₂)cycloalkenyl-Ar, wherein the alkyl, alkenyl, or alkynyl is optionally substituted with one or more groups independently selected from —P(O)OH(OR⁵⁰), —O—P(O)OH(OR⁵¹), SO₃H, —C(O)NH(R⁴⁰), (C₁-C₆)alkyl-OH, —OH, or —C(O)OH;
   Ar is a ring having the structure:

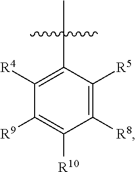

wherein:
R⁴ and R⁵ are each independently —H, —CO₂H, halo, cyano, —OH, (C₁-C₆)alkyl-OH, —SO₃H, nitro, triflate, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl, —C(O)NH(R⁴⁰), —P(O)(OR⁵⁰)₂, —O—P(O)(OR⁵¹)₂, or (C₃-C₈)cyclic amino;
each R⁴⁰ is independently —H, cyano, or SO₂(R⁴¹);
each R⁴¹ is independently (C₁-C₆)alkyl, NH₂, NH((C₁-C₆)alkyl), or N((C₁-C₆)alkyl)₂; each R⁵⁰ is independently-H, (C₁-C₆)alkyl, or acetoxymethyl;
each R⁵¹ is independently-H, (C₁-C₆)alkyl, or acetoxymethyl;
R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R⁸⁰;
   each R⁸⁰ is independently selected from oxo, halo, —CO₂H, —C(O)O—N-succinimide, maleimido, azido, propargyl, norbornenyl or tetrazinyl, or a sensor or targeting group;
R⁹ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; and
R¹⁰ —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R¹⁰⁰;
   each R¹⁰⁰ is independently selected from oxo, halo, —CO₂H, —C(O)O—N-succinimide, maleimido, azido, propargyl, norbornenyl, or tetrazinyl, or a sensor or targeting group; or
Ar is a ring having the structure:

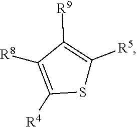

wherein one of R⁴, R⁵, R⁸, and R⁹ is covalently attached to X, and the rest of R⁴, R⁵, R⁸, and R⁹ are as defined above; and
R⁶ is (C₁-C₂₅)aliphatic, (C₂-C₂₅)heteroaliphatic substituted with a leaving group;
R⁷ is (C₁-C₂₅)aliphatic, (C₂-C₂₅)heteroaliphatic, (C₆-C₁₅)aryl or (C₅-C₁₅)heteroaryl,
wherein:
   the aliphatic and heteroaliphatic of R⁶ and R⁷ are optionally substituted with one or more R⁶⁰, and the aryl or heteroaryl of R⁷ is optionally substituted with one or more R⁶¹
   each R⁶⁰ is independently selected from oxo, halo, —CO₂H, —C(O)O—N-succinimide, maleimido, amino, hydroxyl, thiol, azido, propargyl, norbornenyl, or tetrazinyl, or a sensor or targeting group; and
   each R⁶¹ is independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H.

Alternative values for the variables are as described in the first through tenth embodiments, or any aspect thereof.

Protecting groups, such as those described herein, are often used to render otherwise chemically incompatible chemical moieties (e.g., substituent(s), functional group(s)) chemically compatible with a particular set of reaction conditions and/or a desired transformation. Accordingly, some aspects of any of the methods described herein further comprise protecting a chemically incompatible chemical moiety(ies) (e.g., substituent(s), functional group(s)) to form a protected chemical moiety(ies) (e.g., substituent(s), functional group(s)). Non-limiting examples of chemical moieties that can conveniently be protected and thereby rendered chemically compatible include hydroxyls, free aminos, aldehydes, thiols and carboxylic acids.

Deprotection of chemically incompatible chemical moiety(ies) (e.g., substituent(s), functional group(s)) results in removal of protecting group(s), and exposure of the original moity(ies). Accordingly, some aspects of any of the methods described herein further comprise deprotecting the protected chemical moiety(ies).

Orthogonal protecting group strategies can be employed when there are two or more chemical moieties in a compound that potentially share common reactivity and it is desired to derivatize or transform one (or more) chemical moiety(ies) independently of the one or more other chemical moiety(ies). Methods for protecting and deprotecting particular functional groups, as well as orthogonal protecting group strategies are known in the art and can be found, for example, in Wuts, P.G.M. Protecting Groups in Organic Synthesis, 5^{th} Ed., New York, John Wiley & Sons, 2014, the entirety of which is incorporated herein by reference.

Examples of suitably protected hydroxyl groups include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl ethers include trimethylsilyl, triethylsilyl, t-butyldimethylsilyl, t-butyldiphenylsilyl, triisopropylsilyl ether, and other trialkylsilyl ethers. Examples of alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Examples of mono-protected aminos include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Di-protected aminos include aminos that are substituted with two substituents independently selected from those described above as mono-protected aminos, and further include cyclic imides, such as phthalimide, maleimide, succinimide, and the like. Di-protected aminos also include pyrroles and the like, 2,2,5,5-tetramethyl-[1,2,5]azadisilolidine and the like, and azide.

Protected aldehydes include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl esters, wherein each group is optionally substituted. Additional protected carboxylic acids include oxazolines and ortho esters.

Protected thiols include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester.

Typically, a reaction (e.g., modification reaction, protection and/or deprotection reaction) described herein is carried out in an appropriate solvent. As used herein, “solvent” refers to a liquid that serves as a medium for a chemical reaction or other procedure in which compounds are being manipulated (e.g., purification). Typically, the solvent in the methods disclosed herein is an organic solvent or water, or a combination thereof. Examples of organic solvents include polar, protic solvents (e.g., an alcohol such as methanol, ethanol, butanol, such as tert-butanol), polar aprotic solvents (e.g., acetonitrile, dimethylformamide, tetrahydrofuran, ethyl acetate, acetone, methyl ethyl ketone) or nonpolar solvents (e.g., diethyl ether).

In some aspects, the leaving group is iodo or chloro.

In some aspects, the nucleophile is a thiol, amine, hydroxyl, phosphine, carbanion, sulfinite, azide, cyano, or phosphite. In some aspects, the nucleophile comprises a sensor, a targeting group or a clickable moiety.

Spirolactonizable Si-rhodamines have been found to be particularly valuable for live cell imaging. Thus, another embodiment is a method of imaging a cell (e.g., a live cell), comprising contacting the cell with a compound of the present disclosure or a tautomer (e.g., a ring-closed tautomer) thereof, or a salt of the foregoing; illuminating the cell; and detecting fluorescence from the cell. Methods of conducting live-cell imaging are known in the art, and are described herein.

Another embodiment is a method of labeling a biomolecule or cell (e.g., in a multicellular organism) comprising contacting the biomolecule or cell with a compound of the present disclosure, or a tautomer (e.g., a ring-closed tautomer) thereof, or a salt of the foregoing, thereby labeling the biomolecule or cell. In embodiments, the biomolecule is a protein, a nucleic acid, or a lipid. In embodiments, the multicellular organism is a mouse, a rat, a zebrafish, or C. elegans.

Yet another embodiment is a method of detecting a target in a sample, comprising contacting the sample with a compound of the present disclosure, or a tautomer (e.g., a ring-closed tautomer) thereof, or a salt of the foregoing, comprising a targeting group for the target; illuminating the sample; and detecting fluorescence from the sample. In some aspects, the sample comprises a cell (e.g., a live cell). Methods of detecting a target in a sample, for example, using fluorescence spectroscopy, are known in the art, and are described herein.

### EXEMPLIFICATION

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon, as appreciated by those skilled in the art. Preferred methods include, but are not limited to, those described below. The reactions are performed in a solvent or solvent mixture appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one particular process scheme over another in order to obtain the desired compound.

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd} ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in view of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

All reactions were performed in oven-dried round bottomed flasks fitted with rubber septa under argon atmosphere, unless otherwise noted. All reagents and solvents, including anhydrous solvents, were purchased from commercial sources and used as received. Flash column chromatography was performed on an ISCO CombiFlash Rf+ instrument using RediSep Gold, Silicycle, or Biotage columns. Thin-layer chromatography (TLC) was performed using silica gel (60 F-254) coated aluminum plates (EMD Millipore), and spots were visualized by exposure to ultraviolet light (UV), exposure to iodine adsorbed on silica gel, and/or exposure to an acidic solution of p-anisaldehyde (anisaldehyde) or phosphomolybdic acid (PMA) followed by brief heating. ¹H NMR and ¹³C NMR spectra were acquired on a Bruker Avance III HD 500 MHz NMR instrument. Chemical shifts are reported in ppm (S scale) with the residual solvent signal used as reference and coupling constant (J) values are reported in hertz (Hz). Data are presented as follows: chemical shift, multiplicity (s=singlet, d=doublet, dd=doublet of doublet, t=triplet, q=quartet, m=multiplet, br s=broad singlet), coupling constant in Hz, and integration. High-resolution mass spectra (HRMS) were recorded on a Thermo Scientific Orbitrap Velos Pro mass spectrometer coupled with a Thermo Scientific Accela 1250 UPLC and an autosampler using electrospray ionization (ESI) in the positive mode. Preparatory HPLC was performed on a Varian ProStar equipped with Agilent 10-Prep C18 21.2×250 mm Column. Small molecule x-ray crystallography was performed at the UMass Dartmouth X-ray Diffraction Facility.

Absorption and fluorescence spectra were measured on a Horiba Duetta fluorescence and absorbance spectrometer in quartz cuvettes (Starna Cells, catalog #3-Q-10). Extinction coefficients were calculated from plots of absorption versus concentration. Quantum yields were measured on a Hamamatsu Quantaurus QY C-11347-11 absolute quantum yield integrating sphere spectrometer at absorption values of <0.1 in side-arm quartz cuvettes (Hamamatsu cat #A10095-02). All measurements were performed in PBS (9.0 g/l NaCl, 0.795 g/l Na₂HPO₄, 0.144 g/l KH₂PO₄, pH 7.4, Corning cat #21-040-CV), ethanol, or 0.1% TFA/ethanol and prepared from stock solutions of dyes in DMSO, with final DMSO <1%. The photophysical properties of 061 and 062 were also measured after a 2 h incubation in 0.1% SDS/PBS and after 2 h treatment with 30 μM hairpin DNA (hpDNA). Selected KLZ values were determined in 1:1 v/v dioxane:water as previously described.

A colorless crystal of 037, recrystallized from 1:1 DCM/EtOAc, was mounted on a Cryoloop with oil. Data were collected at 24° C. on a Bruker D8 Venture X-ray single crystal instrument using Mo K alpha radiation and data were corrected for absorption with SADAS. The structure was solved by direct methods (intrinsic phasing), and all non-hydrogen atoms were refined by full matrix least squares on F². All hydrogen atoms were placed in calculate positions with appropriate riding parameters.

### Synthetic Methods

Rapid access to novel Si-substituted dyes, began with dibromo scaffold 1-1 (Scheme 1A). Lithium-halogen exchange chemistry followed by reaction with different commercially-available dichlorosilanes afforded the Si-leuco dyes, which were directly oxidized with p-chloranil to yield the desired Si-rhodamines, purified as the TFA salt

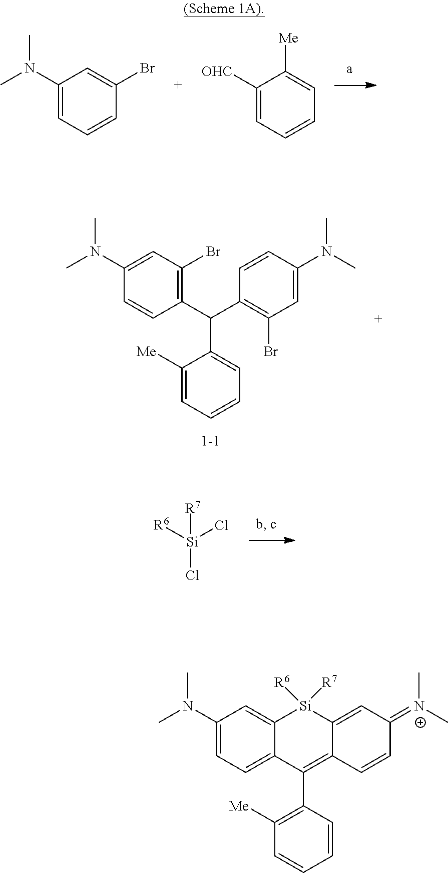

Scheme 1A. (a) p-toluenesulfonic acid (PTSA), toluene, 135° C., overnight, 52%; (b) s-BuLi (1.4M in cyclohexane), THF, −78° C. to RT, RT 12 h; (c) p-Chloranil, DCM, RT, 2 h.

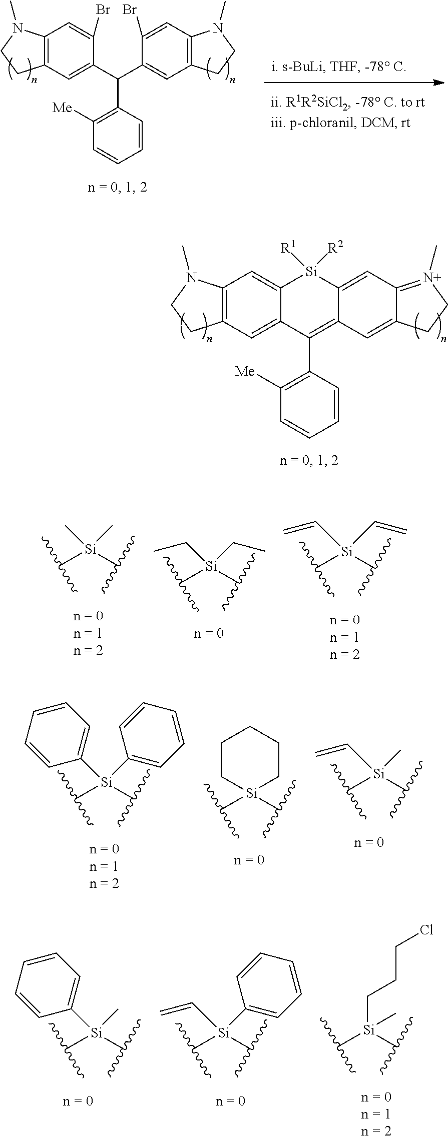

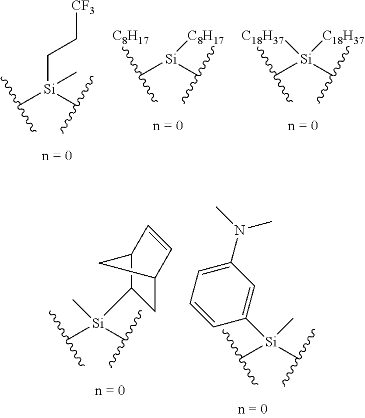

Scheme 1B. Synthesis of Si-bridge rhodamines, wherein R¹ and R² in Scheme 1B correspond to R⁶ and R⁷, respectively, in the compounds of Formula (I).

Gratifyingly, most dichlorosilanes yielded the expected Si-rhodamine dye, with the exception of cyclobutyl and cyclopentyl dichlorosilanes, which form strained and likely unstable reaction products. Asymmetrically substituted Si-dyes led to two isomers that were evident by NMR but not separated by chromatography.

Simple six-membered and five-membered simple siloles are known to be stable in solution. However, silacyclopentyl Si-rhodamine dyes were not stable in solution, in contrast with prior synthesized simple silole compounds. Thus, simple siloles are not predictive of success in Si-rhodamine dyes.

### Example 1: 4,4′-(o-Tolylmethylene)bis(3-bromo-N,N-dimethylaniline) (1-1)

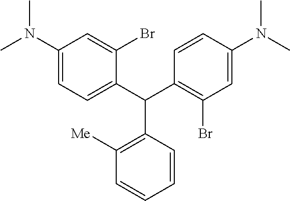

A solution of 3-bromo-N,N-dimethylaniline (8.0 g, 40.0 mmol) in anhydrous toluene (20.0 mL) was treated with 2-methylbenzaldehyde (1.16 mL, 10.0 mmol) and p-toluenesulfonic acid monohydrate (1.90 g, 10.0 mmol) at room temperature and then the mixture solution was refluxed in a Dean-Stark apparatus. After 3 h, another 20 mL of anhydrous toluene was added into the reaction and the reaction mixture was refluxed overnight, then cooled to room temperature. The solvent was evaporated under reduced pressure, and the residue was dried under high vacuum for an hour. The resulting oil was then dissolved in dichloromethane (200 mL), washed with saturated aqueous NaHCO₃ (100 mL), dried (Na₂SO₄), filtered, and evaporated under reduced pressure. The residue was purified by flash column chromatography (RediSep Gold column, 80 g, gradient elution with 0-60% DCM/hexanes) to provide 1 (2.60 g, 52%) as a white foamy solid. ¹H NMR (500 MHz, CDCl₃) δ 7.17-7.11 (m, 2H), 7.07 (td, J=7.5, 2.5 Hz, 1H), 6.94 (d, J=2.5 Hz, 2H), 6.72 (d, J=7.5 Hz, 1H), 6.62 (d, J=9.0 Hz, 2H), 6.53 (dd, J=8.5, 2.5 Hz, 2H), 5.96 (s, 1H), 2.91 (s, 12H), 2.20 (s, 3H) ppm; ¹³C NMR (125 MHz, CDCl₃) δ 150.04, 141.75, 137.12, 131.09, 130.37, 129.63, 128.85, 126.65, 126.35, 125.72, 116.60, 111.34, 51.52, 40.53, 19.67 ppm; IRMS (ESI) m/z: [M+H]⁺ calcd for C₂₄H₂₇Br₂N₂, 503.0515; found 503.0509.

### Example 2: N-(7-(Dimethylamino)-5,5-dimethyl-10-(o-tolyl)dibenzo[b,e]silin-3(5H)-ylidene)-N-methylmethanaminium (001)

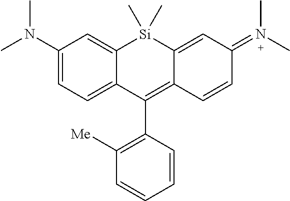

A degassed solution of 4,4′-(o-tolylmethylene)bis(3-bromo-N,N-dimethylaniline) (0.10 g, 0.20 mmol) in anhydrous THE (5.0 mL) under argon atmosphere was cooled to −78° C. in an acetone/dry ice bath. After 15 min, s-BuLi (1.4M in cyclohexane) (0.32 mL, 0.44 mmol) was added dropwise over 10 min. The resulting reaction mixture was stirred at −78° C. for additional one hour. At the same temperature, dichlorodimethylsilane (40.0 μL, 0.30 mmol) dissolved in anhydrous THE (5.0 mL) was added dropwise over 10 min. The reaction mixture was then slowly warmed to room temperature and stirred overnight. The reaction mixture was then cooled to ˜5° C. and quenched by addition of 2 N HCl (1.0 mL) and stirred at room temperature for 10 min. NaHCO₃ (10.0 mL) was added and then extracted with dichloromethane (25.0 mL), which was dried (Na₂SO₄), filtered, and evaporated under reduced pressure. The residue was redissolved in anhydrous DCM (10.0 mL) and treated with p-chloranil (0.10 g, 0.40 mmol) at room temperature, and then the mixture solution was stirred for 2 h. The solvent was then evaporated under reduced pressure, and the residue purified by flash column chromatography (Silicycle column, 12 g, 0-15% MeOH in 1% v/v TFA/DCM, linear gradient for 20 minutes) to yield (60.0 mg, 68%) of the trifluoroacetate salt of 001 as a dark blue color solid. An analytically pure (>99%) sample was obtained through further purification by reverse-phase HPLC (30-95% MeCN/H₂O, linear gradient, with constant 0.1% v/v TFA additive); this material (the TFA salt) was used for all characterization purposes. ¹H NMR (500 MHz, CDCl₃) δ 7.45-7.41 (m, 1H), 7.36-7.32 (m, 2H), 7.18 (d, J=2.5 Hz, 2H), 7.10-7.07 (m, 3H), 6.59 (dd, J=10.0, 3.0 Hz, 2H), 3.43 (s, 12H), 2.03 (s, 3H), 0.60 (s, 3H), 0.58 (s, 3H) ppm; ¹³C NMR (125 MHz, CDCl₃) δ 170.44, 154.29, 148.83, 141.77, 138.54, 135.83, 130.41, 129.08, 129.02, 127.79, 125.78, 120.88, 114.03, 40.97, 19.50, −0.84, −1.17 ppm; ¹⁹F NMR (470 MHz, CDCl₃); −75.65 ppm; IRMS (ESI) m/z: [M]⁺ calcd for C₂₆H₃₁N₂Si, 399.2251; found 399.2243.

### Example 3: N-(7-(Dimethylamino)-5,5-diethyl-10-(o-tolyl)dibenzo[b,e]silin-3(5H)-ylidene)-N-methylmethanaminium (002)

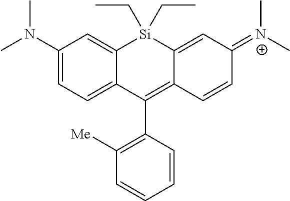

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorodiethylsilane (70.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 002 (90.0 mg, 71%) as a dark blue color solid. ¹H NMR (500 MHz, CDCl₃) δ 7.46-7.40 (m, 1H), 7.38-7.30 (m, 2H), 7.13 (d, J=2.0 Hz, 2H), 7.12-7.06 (m, 3H), 6.62 (dd, J=9.5, 2.5 Hz, 2H), 3.34 (s, 12H), 2.01 (s, 3H), 1.14-1.03 (m, 4H), 1.00 (t, J=8.0 Hz, 3H), 0.93 (t, J=7.5 Hz, 3H) ppm; ¹³C NMR (125 MHz, CDCl₃) δ 170.99, 154.12, 146.90, 141.90, 138.53, 135.73, 130.43, 129.11, 129.01, 128.58, 125.78, 120.61, 114.13, 40.93, 19.43, 7.31, 7.21, 6.32, 5.78 ppm; ¹⁹F NMR (470 MHz, CDCl₃); −75.72 ppm; HRMS (ESI) m/z: [M]⁺ calcd for C₂₈H₃₅N₂Si, 427.2564; found 427.2557.

### Example 4: N-(7-(Dimethylamino)-5-methyl-10-(o-tolyl)-5-vinyldibenzo[b,e]silin-3(5H)-ylidene)-N-methylmethanaminium (003)

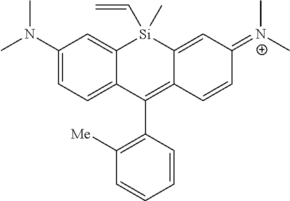

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(methyl)(vinyl)silane (63.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 003 as an inseparable mixture of diastereomers (72.0 mg, 58%) in dark blue color solid. ¹H NMR (500 MHz, CDCl₃) δ 7.46-7.41 (m, 1H), 7.37-7.31 (m, 2H), 7.13-7.06 (m, 5H), 6.61 (dd, J=9.5, 3.0 Hz, 2H), 6.37-6.21 (m, 2H), 6.00 (dd, J=19.5, 3.5 Hz, 1H), 3.33 (s, 12H), 2.04 and 2.01 (2×s, 3H), 0.68 and 0.65 (2×s, 3H) ppm; ¹³C NMR (125 MHz, CDCl₃) δ 170.55, 154.25, 146.46, 141.89, 138.35, 138.01, 137.69, 135.81 (2 signals), 133.06, 132.72, 130.45 (2 signals), 129.15, 129.05, 128.97, 127.95, 125.80 (2 signals), 121.53 (2 signals), 114.18, 40.94, 19.49 (2 signals), −3.26 (2 signals) ppm; ¹⁹F NMR (470 MHz, CDCl₃); −75.63 ppm; HRMS (ESI) m/z: [M]⁺ calcd for C₂₇H₃₁N₂Si, 411.2251; found 411.2244.

### Example 5: N-(7-(Dimethylamino)-5-methyl-5-phenyl-10-(o-tolyl)dibenzo[b,e]silin-3(5H)-ylidene)-N-methylmethanaminium (004)

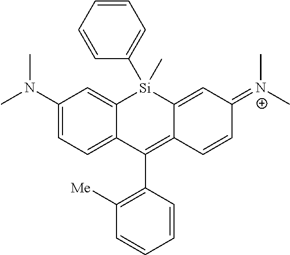

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(methyl)(phenyl)silane (85.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 004 as an inseparable mixture of diastereomers (0.11 g, 76%) in dark blue color solid. ¹H NMR (500 MHz, CDCl₃) δ 7.59-7.54 (m, 2H), 7.51-7.40 (m, 4H), 7.39-7.32 (m, 2H), 7.15-7.09 (m, 3H), 7.08-7.05 (m, 2H), 6.61 (dd, J=9.5, 2.5 Hz, 2H), 3.26 (s, 12H), 2.07 and 2.05 (2×s, 3H), 0.93 and 0.90 (2×s, 3H) ppm; ¹³C NMR (125 MHz, CDCl₃) δ 170.73, 154.29 (2 signals), 146.83, 141.90 (2 signals), 138.34, 135.91, 134.66, 134.53, 133.34, 131.00, 130.50, 129.22, 128.94, 128.89, 128.10 (2 signals), 125.84 (2 signals), 121.71, 114.25, 40.89, 19.50 (2 signals), −3.25 (2 signals) ppm; ¹⁹F NMR (470 MHz, CDCl₃); −75.85 ppm; HRMS (ESI) m/z: [M]⁺ calcd for C₃₁H₃₃N₂Si, 461.2408; found 461.2401.

### Example 6: N-(7-(Dimethylamino)-5-phenyl-10-(o-tolyl)-5-vinyldibenzo[b,e]silin-3(5H)-ylidene)-N-methylmethanaminium (005)

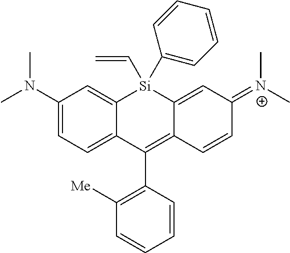

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(phenyl)(vinyl)silane (92.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 005 as an inseparable mixture of diastereomers (0.12 g, 80%) in dark blue color solid. ¹H NMR (500 MHz, CDCl₃) δ 7.62-7.48 (m, 3H), 7.47-7.41 (m, 3H), 7.38-7.31 (m, 2H), 7.16-7.05 (m, 5H), 6.63 (dd, J=10.0, 3.0 Hz, 2H), 6.58-6.42 (m, 2H), 5.95 (dd, J=19.5, 3.5 Hz, 1H), 3.28 (s, 12H), 2.04 and 2.01 (2×s, 3H) ppm; ¹³C NMR (125 MHz, CDCl₃) δ 170.59, 154.17 (2 signals), 144.72 (2 signals), 142.08, 139.97, 138.16, 135.88, 135.41, 135.23, 131.25, 131.10, 131.00, 130.50, 129.24, 128.96, 128.24, 125.81 (2 signals), 122.45, 114.36, 40.91, 19.45 (2 signals) ppm; ¹⁹F NMR (470 MHz, CDCl₃); −75.77 ppm; IRMS (ESI) m/z: [M]⁺ calcd for C₃₂H₃₃N₂Si, 473.2408; found 473.2401.

### Example 7: N-(7-(Dimethylamino)-10-(o-tolyl)-5,5-divinyldibenzo[b,e]silin-3(5H)-ylidene)-N-methylmethanaminium (006)

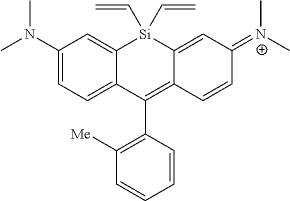

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorodivinylsilane (70.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-15% MeOH in 1% v/v TFA/DCM, linear gradient for 20 min) to yield (70.0 mg, 52%) of the trifluoroacetate salt of 006 as a dark blue color solid. An analytically pure sample was obtained through further purification by reverse-phase HPLC (30-95% MeCN/H2O, linear gradient, with constant 0.1% v/v TFA additive). ¹H NMR (500 MHz, CDCl₃) δ 7.46-7.41 (m, 1H), 7.36-7.32 (m, 2H), 7.12-7.07 (m, 5H), 6.62 (dd, J=9.5, 3.0 Hz, 2H), 6.44-6.28 (m, 4H), 6.02 (dd, J=19.5, 3.5 Hz, 1H), 5.94 (dd, J=18.0, 5.0 Hz, 1H), 3.33 (s, 12H), 2.07 (s, 3H) ppm; ¹³C NMR (125 MHz, CDCl₃) δ 170.39, 154.18, 144.32, 142.00, 139.66, 139.25, 138.22, 135.83, 131.05, 130.68, 130.47, 129.19, 129.04, 128.13, 125.80, 122.23, 114.32, 40.98, 19.48 ppm; ¹⁹F NMR (470 MHz, CDCl₃); −75.64 ppm; HRMS (ESI) m/z: [M]⁺ calcd for C₂₈H₃₁N₂Si, 423.2251; found 423.2244.

### Example 8: N-(7-(Dimethylamino)-5,5-diphenyl-10-(o-tolyl)dibenzo[b,e]silin-3(5H)-ylidene)-N-methylmethanaminium (007)

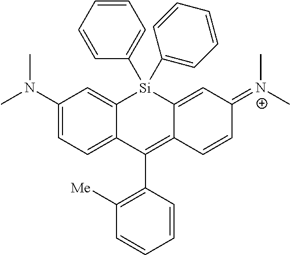

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorodiphenylsilane (94.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-15% MeOH in 1% v/v TFA/DCM, linear gradient for 20 min) to yield (80.0 mg, 51%) of the trifluoroacetate salt of 007 as a dark blue color solid. An analytically pure sample was obtained through further purification by reverse-phase HPLC (30-95% MeCN/H₂O, linear gradient, with constant 0.1% v/v TFA additive). ¹H NMR (500 MHz, CDCl₃) δ 7.64 (dd, J=8.0, 1.0 Hz, 2H), 7.58 (dd, J=8.0, 1.0 Hz, 2H), 7.56-7.51 (m, 2H), 7.50-7.43 (m, 5H), 7.37-7.33 (m, 2H), 7.15 (d, J=9.5 Hz, 2H), 7.13-7.10 (m, 3H), 6.65 (dd, J=10.0, 3.0 Hz, 2H), 3.25 (s, 12H), 2.09 (s, 3H) ppm; ¹³C NMR (125 MHz, CDCl₃) δ 170.46, 154.15, 145.16, 142.09, 138.14, 135.98, 135.82, 131.44, 131.39, 131.32, 130.89, 130.54, 129.28, 129.06, 129.03, 129.01, 128.43, 125.85, 122.74, 114.49, 40.96, 19.48 ppm; ¹⁹F NMR (470 MHz, CDCl₃); −75.66 ppm; HRMS (ESI) m/z: [M]⁺ calcd for C₃₆H₃₅N₂Si, 523.2564; found 523.2552.

### Example 9: N-(7-(Dimethylamino)-5-(3-(dimethylamino)phenyl)-5-methyl-10-(o-tolyl)dibenzo[b,e]silin-3(5H)-ylidene)-N-methylmethanaminium (008)

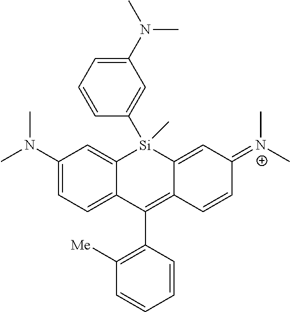

A degassed solution of 3-bromo-N,N-dimethylaniline (1.37 g, 6.82 mmol) in anhydrous Et₂O (20.0 mL) under argon atmosphere was cooled to −78° C. in an acetone/dry ice bath. After 15 min, s-BuLi (1.4M in cyclohexane) (5.35 mL, 7.16 mmol) was added dropwise over 10 min. The resulting reaction mixture was stirred at −78° C. for additional one hour and was then added dropwise to a solution of methyltrichlorosilane (4.02 mL, 34.1 mmol) in anhydrous Et2O (20.0 mL) under argon atmosphere at −78° C. The resulting reaction mixture was then slowly warmed to room temperature and stirred for 2 h. The resulting reaction mixture was filtered through Celite and concentrated under reduced pressure. The crude dichloro(methyl)(3-(dimethylamino)phenyl)silane was used without further purification to prepare 008.

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(methyl)(3-(dimethylamino)phenyl)silane (0.11 g, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in DCM for 10 min, hold at 5% MeOH isocratic for 5 min, and then increase to 15% gradient over 5 min) to provide 008 as an inseparable mixture of diastereomers (50.0 mg, 34%) in dark blue color solid. ¹H NMR (500 MHz, CDCl₃) δ 7.45-7.40 (m, 1H), 7.37-7.27 (m, 3H), 7.14-7.06 (m, 5H), 6.92-6.78 (m, 3H), 6.66 (dd, J=9.5, 3.0 Hz, 2H), 3.34 (s, 12H), 2.95 and 2.92 (2×s, 6H), 2.06 and 2.05 (2×s, 3H), 0.93 and 0.89 (2×s, 3H) ppm; ¹³C NMR (125 MHz, CDCl₃) δ 170.03, 154.23 (2 signals), 147.01, 141.70 (2 signals), 138.46, 135.85, 133.62, 131.00, 130.46 (2 signals), 129.62, 129.12, 128.95, 128.04, 125.85 (2 signals), 122.41, 121.70, 117.79, 114.90, 114.39, 41.22, 40.63, 19.59 (2 signals), −2.79 (2 signals) ppm; HRMS (ESI) m/z: [M]⁺ calcd for C₃₃H₃₈N₃Si, 504.2830; found 504.2824.

### Example 10: N-(5-(3-Chloropropyl)-7-(dimethylamino)-5-methyl-10-(o-tolyl)dibenzo[b,e]silin-3(5H)-ylidene)-N-methylmethanaminium (009)

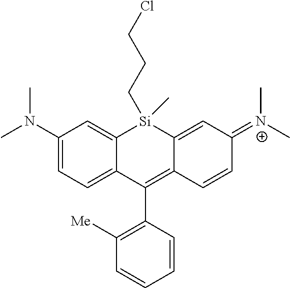

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(3-chloropropyl)(methyl)silane (70.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-15% MeOH in 1% v/v TFA/DCM, linear gradient for 20 min) to yield (70.0 mg, 50%) of the trifluoroacetate salt of 009 as an inseparable mixture of diastereomers in dark blue color solid. An analytically pure sample was obtained through further purification by reverse phase HPLC (30-95% MeCN/H2O, linear gradient, with constant 0.1% v/v TFA additive). ¹H NMR (500 MHz, CDCl₃) δ 7.46-7.41 (m, 1H), 7.37-7.32 (m, 2H), 7.19 (t, J=3.0 Hz, 2H), 7.11-7.06 (m, 3H), 6.60 (dd, J=9.5, 2.5 Hz, 2H), 3.47 and 3.43 (2×t, J=7.0 Hz, 2H), 3.35 (s, 12H), 2.03 and 2.02 (2×s, 3H), 1.77-1.63 (m, 2H), 1.22-1.16 (m, 2H), 0.67 and 0.65 (2×s, 3H) ppm; ¹³C NMR (125 MHz, CDCl₃) δ 170.47 (2 signals), 154.24 (2 signals), 147.14 (2 signals), 141.80 (2 signals), 138.46 (2 signals), 135.92, 135.66, 130.44 (2 signals), 129.18, 129.13, 129.12, 128.12, 128.02, 125.83 (2 signals), 121.06 (2 signals), 114.17, 47.39 (2 signals), 41.01, 26.99 (2 signals), 19.52, 13.68 (2 signals), −3.37 (2 signals) ppm; ¹⁹F NMR (470 MHz, CDCl₃); −75.67 ppm; HRMS (ESI) m/z: [M]⁺ calcd for C₂₈H₃₄ClN₂Si, 461.2174; found 461.2168.

### Example 11: N-(7-(Dimethylamino)-5-methyl-10-(o-tolyl)-5-(3,3,3-trifluoropropyl)dibenzo[b,e]silin-3(5H)-ylidene)-N-methylmethanaminium (010)

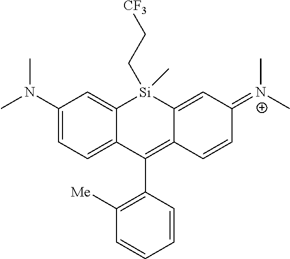

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(3,3,3-trifluoropropyl)(methyl)silane (95.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 010 as an inseparable mixture of diastereomers (90.0 mg, 63%) in dark blue color solid. ¹H NMR (500 MHz, CDCl₃) δ 7.45-7.39 (m, 1H), 7.37-7.29 (m, 2H), 7.20-7.12 (m, 2H), 7.10-7.02 (m, 3H), 6.62 (dd, J=9.5, 2.5 Hz, 2H), 3.33 (s, 12H), 2.01 and 2.00 (2×s, 3H), 1.99-1.84 (m, 2H), 1.30-1.17 (m, 2H), 0.70 and 0.69 (2×s, 3H) ppm; ¹³C NMR (125 MHz, CDCl₃) δ 170.43 (2 signals), 154.25 (2 signals), 145.58 (2 signals), 141.80 (2 signals), 140.15, 138.19 (2 signals), 135.87, 135.38, 130.45, 130.40, 129.15 (2 signals), 128.65, 127.91, 127.80, 127.25 (q, J=269.3 Hz), 125.81, 120.92 (2 signals), 114.34, 40.92 (2 signals), 28.14 (2×q, J=30.2 Hz), 19.36 (2 signals), 8.49 (2 signals), −3.92 (2 signals) ppm; ¹⁹F NMR (470 MHz, CDCl₃); −68.14, −68.23, −75.65 ppm; HRMS (ESI) m/z: [M]⁺ calcd for C₂₈H₃₂F₃N₂Si, 481.2281; found 481.2273.

### Example 12: N-(7-(Dimethylamino)-5,5-dioctyl-10-(o-tolyl)dibenzo[b,e]silin-3(5H)-ylidene)-N-methylmethanaminium (011)

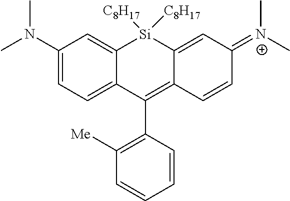

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorodioctylsilane (0.16 mL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-15% MeOH in 1% v/v TFA/DCM, linear gradient for 20 min) to yield (0.10 g, 56%) of the trifluoroacetate salt of 011 as a dark blue color solid. An analytically pure sample was obtained through further purification by reverse-phase HPLC (30-95% MeCN/H2O, linear gradient, with constant 0.1% v/v TFA additive). ¹H NMR (500 MHz, CDCl₃) δ 7.46-7.41 (m, 1H), 7.37-7.31 (m, 2H), 7.13-7.04 (m, 5H), 6.62 (dd, J=10.0, 3.0 Hz, 2H), 3.34 (s, 12H), 2.00 (s, 3H), 1.29-1.12 (m, 24H), 1.10-1.04 (m, 4H), 0.87-0.81 (m, 6H) ppm; ¹³C NMR (125 MHz, CDCl₃) δ 170.96, 154.07, 147.58, 141.95, 138.51, 135.70, 130.46, 129.16, 128.97, 128.41, 125.81, 120.58, 114.16, 40.91, 33.18, 33.10, 31.94, 29.28, 29.22, 29.20, 23.65, 23.60, 22.76, 22.75, 19.43, 14.60, 14.20, 14.19, 14.16 ppm; ¹⁹F NMR (470 MHz, CDCl₃); −75.72 ppm; HRMS (ESI) m/z: [M]⁺ calcd for C₄₀H₅₉N₂Si, 595.4442; found 595.4430.

### Example 13: N-(7-(Dimethylamino)-5,5-dioctadecyl-10-(o-tolyl)dibenzo[b,e]silin-3(5H)-ylidene)-N-methylmethanaminium (012)

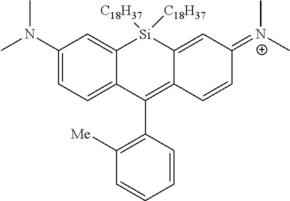

A degassed solution of tetrachlorosilane (1.48 g, 8.73 mmol) in anhydrous Et₂O (20.0 mL) under argon atmosphere was cooled to −78° C. in an acetone/dry ice bath. After 15 min, 0.5M solution of octadecylmagnesium chloride in THE (35.0 mL, 17.5 mmol) was added dropwise over 20 min. The resulting reaction mixture was then slowly warmed to room temperature and stirred for 2 h. The resulting reaction mixture was filtered through celite and concentrated under reduced pressure. The crude dichlorodioctadecylsilane was used without further purification to prepare 012.

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorodioctadecylsilane (0.27 mL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 012 (26.0 mg, 10%) as a dark blue color solid. ¹H NMR (500 MHz, CDCl₃) δ 7.47-7.42 (m, 1H), 7.37-7.32 (m, 2H), 7.14-7.04 (m, 5H), 6.63 (dd, J=10.0, 3.0 Hz, 2H), 3.33 (s, 12H), 2.00 (s, 3H), 1.30-1.04 (m, 64H), 0.90-0.85 (m, 10H) ppm; ¹³C NMR (125 MHz, CDCl₃) δ 171.03, 154.07, 147.59, 141.98, 138.49, 135.69, 132.65, 130.46, 129.18, 128.98, 128.43, 125.82, 120.57, 114.16, 40.93, 33.22, 33.16, 32.07, 29.89, 29.85, 29.80, 29.69, 29.64, 29.50, 29.31, 23.68, 23.64, 22.84, 19.44, 14.63, 14.26, 14.17 ppm; ¹⁹F NMR (470 MHz, CDCl₃); −75.84 ppm; HRMS (ESI) m/z: [M]⁺ calcd for C₆₀H₉₉N₂Si, 875.7572; found 875.7550.

### Example 14: N-(5-((2S)-Bicyclo[2.2.1]hept-5-en-2-yl)-7-(dimethylamino)-5-methyl-10-(o-tolyl)dibenzo[b,e]silin-3(5H)-ylidene)-N-methylmethanaminium (013)

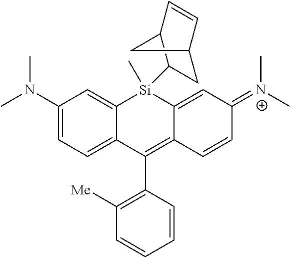

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichloro(methyl)(5-bicyclo[2.2.1]hept-5-en-2-yl)silane (93.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 013 as an inseparable mixture of endo and exo isomers (80.0 mg, 56%) in dark blue color solid. ¹H NMR (500 MHz, CDCl₃) (mixture of endo and exo isomers) δ 7.46-7.41 (m, 1H), 7.37-7.31 (m, 2H), 7.15-7.02 (m, 5H), 6.64-6.57 (m, 2H), 6.15-5.64 (m, 2H), 3.34 (2×s, 12H), 2.99-2.67 (m, 2H), 2.11-1.97 (m, 3H), 1.61-1.02 (m, 4H), 0.77-0.67 (m, 3H), 0.65-0.55 (m, 1H) ppm; ¹⁹F NMR (470 MHz, CDCl₃); −75.81 ppm; HRMS (ESI) m/z: [M]⁺ calcd for C₃₂H₃₇N₂Si, 477.2721; found 477.2714.

### Example 15: N-(7-(Dimethylamino)-10-(o-tolyl)-3H-spiro[dibenzo[b,e]siline-5,1′-silinan]-3-ylidene)-N-methylmethanaminium (014)

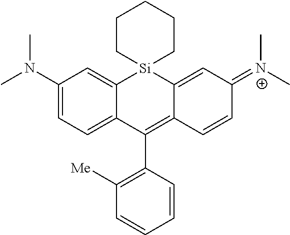

The same procedure was used as described above for compound 001. 4,4′-(o-tolylmethylene)bis(3-bromo-N,N-dimethylaniline) 1-1 (0.15 g, 0.30 mmol) in anhydrous THE (8.0 mL) was treated with s-BuLi (1.4M in cyclohexane) (0.47 mL, 0.66 mmol) and dichlorocyclohexylsilane (80.0 μL, 0.45 mmol). The resulting residue was redissolved in DCM (10.0 mL), followed by treatment with p-chloranil (0.15 g, 0.60 mmol). The residue was purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to provide 014 (50.0 mg, 39%) as a dark blue color solid. ¹H NMR (500 MHz, CDCl₃) δ 7.45-7.40 (m, 1H), 7.36-7.30 (m, 4H), 7.11-7.04 (m, 3H), 6.67 (dd, J=9.5, 3.0 Hz, 2H), 3.42 (s, 12H), 2.08-2.02 (m, 4H), 2.01 (s, 3H), 1.83-1.76 (m, 2H), 1.17-1.09 (m, 4H) ppm; ¹³C NMR (125 MHz, CDCl₃) δ 170.04, 153.97, 148.13, 141.90, 138.56, 135.78, 130.43, 129.08, 129.02, 128.02, 125.81, 121.23, 114.29, 41.28, 29.12, 24.38, 24.30, 19.59, 13.07, 12.87 ppm; HRMS (ESI) m/z: [M]⁺ calcd for C₂₉H₃₅N₂Si, 439.2564; found 439.2556.

Norbornenes are used for inverse-electron demand Diels-Alder (IEDDA) click chemistry with tetrazines. Norbornene-functionalized dye compound 013 (Scheme 2A) was synthesized as a mixture of four isomers (exo/endo norbornene and two atropisomers). It was found that the norbornene dye isomers react with tetrazines under mild conditions (Scheme 2A).

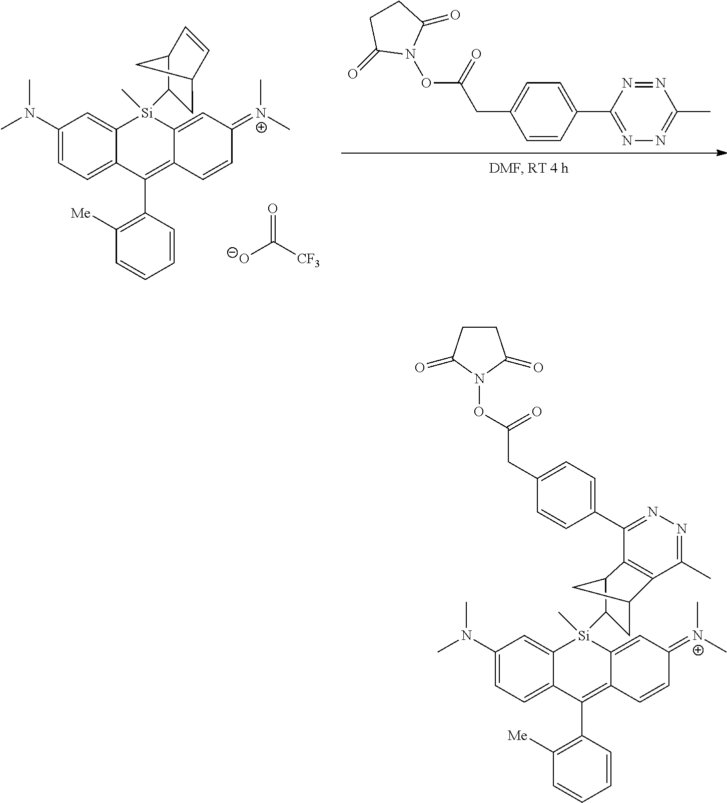

Scheme 2A. Inverse-electron demand Diels-Alder, DMF RT, 4 h.

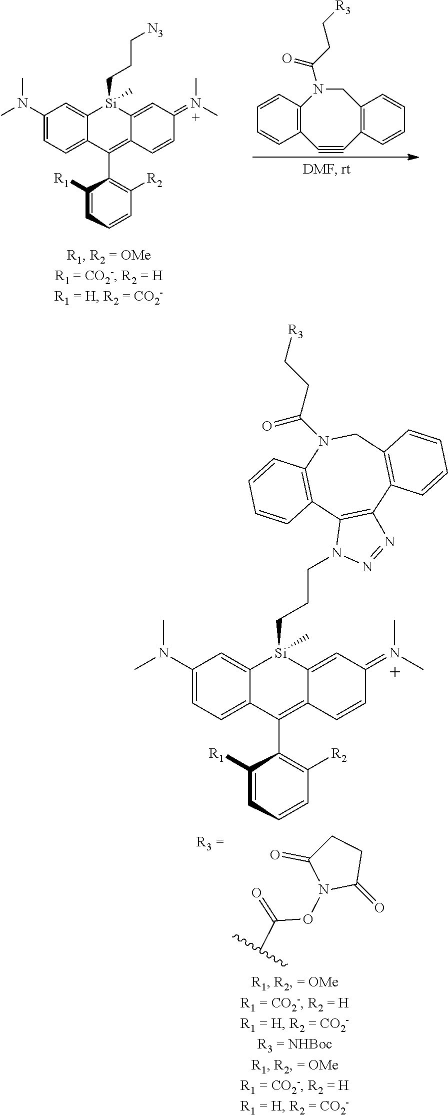

Scheme 2B. Applications of clickable dyes with DBCO NHS ester/NHBoc reagents.

Although the NMR is difficult to interpret due to the presence of multiple isomers, HRMS indicates that the expected cycloaddition products are formed, consistent with the oxidized aromatic pyridazine product rather than the dihydropyridazine (Scheme 2A).

### Example 16: N-(7-(Dimethylamino)-5-((6S)-4-(4-(2-((2,5-dioxopyrrolidin-1-yl)oxy)-2-oxoethyl)phenyl)-1-methyl-5,6,7,8-tetrahydro-5,8-methanophthalazin-6-yl)-5-methyl-10-(o-tolyl)dibenzo[b,e]silin-3(5H)-ylidene)-N-methylmethanaminium (015)

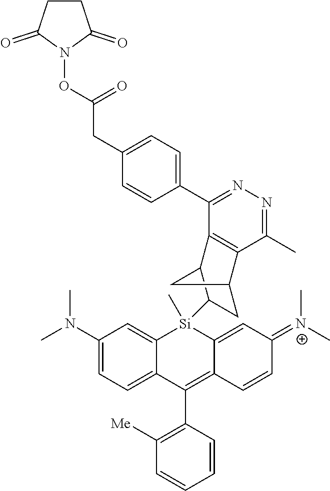 [SMILES: Cc1ccccc1C1=C2C=CC(=[N+](C)C)C=C2[Si](C)(C2CC3CC2c2c(-c4ccc(CC(=O)ON5C(=O)CCC5=O)cc4)nnc(C)c23)c2cc(N(C)C)ccc21]

A solution of 013 (18.0 mg, 0.031 mmol) in anhydrous DMF (0.5 mL) under argon atmosphere was treated with methyltetrazine-NHS ester (12.0 mg, 0.037 mmol) at room temperature and reaction mixture was stirred at room temperature for 4 h. After completion of the reaction, solvent was evaporated under reduced pressure. The resulting residue was purified by flash column chromatography (Biotage column, 10 g, gradient elution with 0-15% MeOH/DCM, linear gradient for 20 min) to provide 015 as an inseparable mixture of exo and endo isomers (20.0 mg, 83%) in dark blue color solid. Although the NMR spectra were not interpretable, HRMS was consistent with the expected product mixture; HRMS (ESI) m/z: [M]⁺ calcd for C₄₇H₄₈N₅O₄Si, 774.3470; found 774.3470.

The effect of divinyl, diphenyl, and chloropropyl silyl groups were studied in a broader range of Si-rhodamine dyes, with different amine donors (Scheme 3).

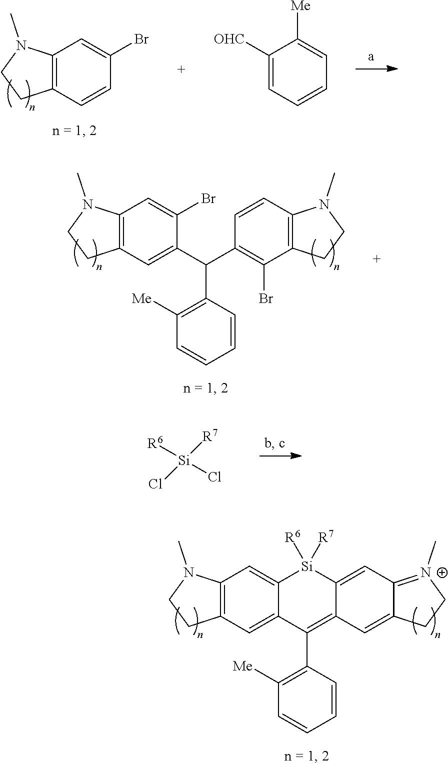

Scheme 3. (a) 2N HCl, reflux, overnight, 60-77%; (b) s-BuLi (1.4M in cyclohexane), THF, −78° C. to RT, 12 h; (c) p-Chloranil, DCM, RT, 2 h, 24-47% over two steps.

### Example 17: 7-Bromo-1-methyl-1,2,3,4-tetrahydroquinoline (2-1)

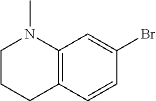

A solution of 7-bromoquinoline (5.0 g, 24.0 mmol) in acetic acid (80.0 mL) was treated with paraformaldehyde (7.21 g, 240 mmol) under an argon atmosphere was cooled to 0° C. in an ice-water bath. After 10 min, NaBH₃CN (3.77 g, 60.0 mmol) was added in small portions. The resulting reaction mixture was warmed to room temperature and stirred for 4 h. The reaction mixture was cooled to 0° C. in an ice-water bath and neutralized with 2M NaOH solution (100 mL). After extraction with DCM (2×150 mL), the combined extracts were washed with saturated NaCl solution (150 mL), dried (Na₂SO₄), filtered, and evaporated under reduced pressure. The resulting residue was purified by flash column chromatography (Silicycle column, 50 g, gradient elution with 0-10% EtOAc/Hexanes) to provide 2-1 (2.90 g, 53%) as a colorless liquid. ¹H NMR (500 MHz, CDCl₃) δ 6.78 (d, J=8.0 Hz, 1H), 6.69 (dd, J=8.0, 2.0 Hz, 1H), 6.66 (d, J=1.5 Hz, 1H), 3.22 (t, J=6.0 Hz, 2H), 2.87 (s, 3H), 2.69 (t, J=6.5 Hz, 2H), 1.95 (p, J=6.5 Hz, 2H) ppm; ¹³C NMR (125 MHz, CDCl₃) δ 147.84, 129.95, 121.62, 120.74, 118.60, 113.37, 51.00, 39.04, 27.50, 22.21 ppm; IRMS (ESI) m/z: [M+H]⁺ calcd for C₁₀H₁₃BrN, 226.0226; found 226.0221.

### Example 18: 6-Bromo-1-methylindoline (3-1)

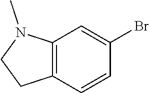

The same procedure was used as described above for compound 2-1. A mixture of 6-bromoindole (6.0 g, 30.6 mmol) and paraformaldehyde (9.19 g, 306 mmol) in AcOH (80.0 mL) was treated with NaBH₃CN (4.80 g, 76.5 mmol) to provide 3-1 (3.80 g, 58%) as a colorless liquid. ¹H NMR (500 MHz, CDCl₃) δ 6.89 (d, J=8.0 Hz, 1H), 6.75 (dd, J=7.5, 1.5 Hz, 1H), 6.55 (d, J=2.0 Hz, 1H), 3.33 (t, J=8.0 Hz, 2H), 2.89 (t, J=8.0 Hz, 2H), 2.74 (s, 3H) ppm; ¹³C NMR (125 MHz, CDCl₃) δ 154.92, 129.39, 125.37, 121.17, 120.17, 110.13, 56.25, 35.79, 28.36 ppm; IRMS (ESI) m/z: [M+H]⁺ calcd for C₉H₁₁BrN, 212.0069; found 212.0067.

### Example 19: 7-Bromo-6-((5-bromo-1-methyl-1,2,3,4-tetrahydroquinolin-6-yl)(o-tolyl)methyl)-1-methyl-1,2,3,4-tetrahydroquinoline (4-1)

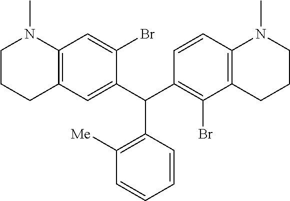 [SMILES: Cc1ccccc1C(c1cc2c(cc1Br)N(C)CCC2)c1ccc2c(c1Br)CCCN2C]

A solution of 7-bromo-1-methyl-1,2,3,4-tetrahydroquinoline 2-1 (1.38 g, 6.10 mmol) in 2N HCl (50.0 mL) was treated with 2-methylbenzaldehyde (0.36 mL, 3.05 mmol) under argon atmosphere was refluxed for overnight. After cooling to room temperature, the reaction mixture was cooled to 0° C. in an ice-water bath and neutralized with saturated NaHCO₃ solution (100 mL) and extraction with DCM (2×100 mL), the combined organic extracts were washed with saturated NaCl solution (100 mL), dried (Na₂SO₄), filtered, and evaporated under reduced pressure. The resulting residue was purified by flash column chromatography (Silicycle column, 50 g, gradient elution with 0-60% DCM/Hexanes) to provide 4-1 (1.30 g, 77%) as a white foamy solid. ¹H NMR (500 MHz, CDCl₃) δ 7.16-7.09 (m, 2H), 7.08-7.04 (m, 1H), 6.78 (s, 2H), 6.73 (d, J=7.5 Hz, 1H), 6.34 (s, 2H), 5.88 (s, 1H), 3.20 (t, J=6.0 Hz, 4H), 2.86 (s, 6H), 2.62-2.51 (m, 4H), 2.20 (s, 3H), 1.91 (p, J=6.0 Hz, 4H) ppm; ¹³C NMR (125 MHz, CDCl₃) δ 141.93, 137.15, 130.73, 130.26, 128.91, 126.19, 125.64, 123.92, 122.02, 114.97, 51.54, 51.06, 39.29, 27.57, 22.15, 19.75 ppm; HRMS (ESI) m/z: [M+H]⁺ calcd for C₂₈H₃₁Br₂N₂, 553.0849; found 553.0839.

### Example 20: 6-Bromo-5-((4-bromo-1-methylindolin-5-yl)(o-tolyl)methyl)-1-methylindoline (5-1)

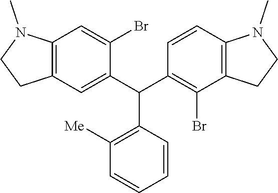 [SMILES: Cc1ccccc1C(c1cc2c(cc1Br)N(C)CC2)c1ccc2c(c1Br)CCN2C]

The same procedure was used as described above for compound 4-1. A solution of 6-bromo-1-methylindoline 3-1 (1.35 g, 6.36 mmol) in 2N HCl (50.0 mL) was treated with 2-methylbenzaldehyde (0.37 mL, 3.19 mmol) to provide 5-1 (1.0 g, 60%) as a white foamy solid. ¹H NMR (500 MHz, CDCl₃) δ 7.18-7.11 (m, 2H), 7.10-7.05 (m, 1H), 6.73 (d, J=7.5 Hz, 1H), 6.68 (s, 2H), 6.49 (s, 2H), 5.96 (s, 1H), 3.32 (t, J=6.5 Hz, 4H), 2.81 (t, J=6.5 Hz, 4H), 2.74 (s, 6H), 2.19 (s, 3H) ppm; ¹³C NMR (125 MHz, CDCl₃) δ 141.84, 137.23, 130.38, 129.92, 128.90, 126.42, 126.37, 125.73, 124.44, 113.96, 56.40, 52.33, 36.42, 28.57, 19.71 ppm; IRMS (ESI) m/z: [M+H]⁺ calcd for C₂₆H₂₇Br₂N₂, 525.0536; found 525.0528.

### Example 21: 1,11,13,13-Tetramethyl-6-(o-tolyl)-2,3,4,8,9,10,11,13-octahydrosilino[3,2-g:5,6-g′]diquinolin-1-ium (016)

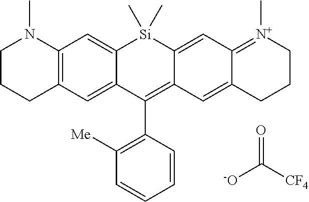

A degassed solution of 4-1 (0.20 g, 0.36 mmol) in anhydrous THE (10.0 mL) under argon atmosphere was cooled to −78° C. in an acetone/dry ice bath. After 15 min, s-BuLi (1.4M in cyclohexane) (0.57 mL, 0.79 mmol) was added dropwise over 5 min. The resulting reaction mixture was stirred at −78° C. for additional 30 min. At the same temperature, dichlorodimethylsilane (57.0 μL, 0.47 mmol) dissolved in anhydrous THE (10.0 mL) was added dropwise over 10 min. The reaction mixture was then slowly warmed to room temperature and stirred overnight. The reaction mixture was then cooled to ˜5° C. and quenched by addition of 2 N HCl (2.0 mL) and stirred at room temperature for 10 min. NaHCO₃ (25.0 mL) was added and then extracted with dichloromethane (50.0 mL), which was dried (Na₂SO₄), filtered, and evaporated under reduced pressure. The residue was redissolved in anhydrous DCM (20.0 mL) and treated with p-chloranil (0.18 g, 0.72 mmol) at room temperature, and then the mixture solution was stirred for 2 h. The solvent was then evaporated under reduced pressure, and the residue purified by flash column chromatography (Silicycle column, 12 g, 0-10% MeOH/DCM, linear gradient, with constant 1% v/v TFA/DCM for 10 min, hold at 5% MeOH isocratic for 5 min, and then increase to 15% gradient over 5 min)) to yield (40.0 mg, 25%) of the trifluoroacetate salt of 016 as a dark blue-green solid. ¹H NMR (500 MHz, CDCl₃) δ 7.45-7.40 (m, 1H), 7.37-7.30 (m, 2H), 7.06 (d, J=7.5 Hz, 1H), 7.01 (s, 2H), 6.66 (s, 2H), 3.58 (t, J=5.5 Hz, 4H), 3.32 (s, 6H), 2.50 (t, J=6.0 Hz, 4H), 2.20 (s, 3H), 1.98-1.89 (m, 4H), 0.55 (s, 3H), 0.53 (s, 3H) ppm; ¹³C NMR (125 MHz, CDCl₃) δ 168.74, 152.05, 147.75, 138.83, 138.36, 135.70, 130.36, 129.06, 128.91, 127.98, 125.75, 124.68, 119.63, 52.70, 39.88, 27.51, 21.00, 19.61, −0.67, −1.11 ppm; ¹⁹F NMR (470 MHz, CDCl₃); −75.85 ppm; HRMS (ESI) m/z: [M]⁺ calcd for C₃₀H₃₅N₂Si, 451.2564; found 451.2559.

### Example 22: 1,11-Dimethyl-6-(o-tolyl)-13,13-divinyl-2,3,4,8,9,10,11,13-octahydrosilino[3,2-g:5,6-g′]diquinolin-1-ium (17)

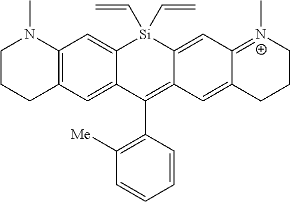 [SMILES: C=C[Si]1(C=C)c2cc3c(cc2C(c2ccccc2C)=c2cc4c(cc21)=[N+](C)CCC4)CCCN3C]

The same procedure was used as described above for compound 016. A solution of 4-1 (0.20 g, 0.36 mmol) in anhydrous THF (10.0 mL) was treated with s-BuLi (1.4M in cyclohexane) (0.57 mL, 0.79 mmol) and dichlorodivinyllsilane (67.0 μL, 0.47 mmol). The resulting residue was re-dissolved in DCM (20.0 mL), followed by treatment with p-chloranil (0.18 g, 0.72 mmol) to provide 017 (45.0 mg, 26%) as a green solid. ¹H NMR (500 MHz, CDCl₃) δ 7.45-7.40 (m, 1H), 7.36-7.30 (m, 2H), 7.06 (d, J=8.0 Hz, 1H), 6.96 (s, 2H), 6.67 (s, 2H), 6.42-6.26 (m, 4H), 6.01 (dd, J=19.5, 3.5 Hz, 1H), 5.93 (dd, J=16.5, 6.0 Hz, 1H), 3.60 (t, J=5.5 Hz, 4H), 3.30 (s, 6H), 2.51 (t, J=6.0 Hz, 4H), 2.02 (s, 3H), 1.99-1.90 (m, 4H) ppm; ¹³C NMR (125 MHz, CDCl₃) δ 168.48, 151.96, 143.18, 139.28, 138.83, 138.60, 138.53, 135.73, 131.44, 131.07, 130.40, 129.10, 128.98, 128.31, 125.75, 124.99, 121.09, 52.78, 39.89, 27.49, 20.94, 19.60 ppm; ¹⁹F NMR (470 MHz, CDCl₃); −75.80 ppm; HRMS (ESI) m/z: [M]⁺ calcd for C₃₂H₃₅N₂Si, 475.2564; found 475.2557.

### Example 23: 1,11-Dimethyl-13,13-diphenyl-6-(o-tolyl)-2,3,4,8,9,10,11,13-octahydrosilino[3,2-g:5,6-g′]diquinolin-1-ium (018)

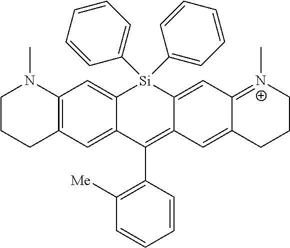 [SMILES: Cc1ccccc1C1=c2cc3c(cc2[Si](c2ccccc2)(c2ccccc2)c2cc4c(cc21)CCCN4C)=[N+](C)CCC3]

The same procedure was used as described above for compound 016. A solution of 4-1 (0.20 g, 0.36 mmol) in anhydrous THF (10.0 mL) was treated with s-BuLi (1.4M in cyclohexane) (0.57 mL, 0.79 mmol) and dichlorodiphenylsilane (0.10 mL, 0.47 mmol). The resulting residue was re-dissolved in DCM (20.0 mL), followed by treatment with p-chloranil (0.18 g, 0.72 mmol) to provide 018 (50.0 mg, 24%) as a green solid. ¹H NMR (500 MHz, CDCl₃) δ 7.67-7.62 (m, 2H), 7.61-7.57 (m, 2H), 7.56-7.42 (m, 7H), 7.38-7.32 (m, 2H), 7.10 (d, J=7.0 Hz, 1H), 6.99 (s, 2H), 6.71 (s, 2H), 3.58 (t, J=5.0 Hz, 4H), 3.17 (s, 6H), 2.52 (t, J=5.5 Hz, 4H), 2.03 (s, 3H), 1.99-1.89 (m, 4H) ppm; ¹³C NMR (125 MHz, CDCl₃) δ 168.50, 151.96, 143.99, 138.56, 135.98, 135.80, 135.73, 131.95, 131.39, 131.23, 131.16, 130.47, 129.13, 129.06, 128.96, 128.94, 128.62, 125.81, 125.17, 121.61, 52.79, 39.85, 27.47, 20.89, 19.62 ppm; ¹⁹F NMR (470 MHz, CDCl₃); −75.83 ppm; HRMS (ESI) m/z: [M]⁺ calcd for C₄₀H₃₉N₂Si, 575.2877; found 575.2871.

### Example 24: 13-(3-Chloropropyl)-1,11,13-trimethyl-6-(o-tolyl)-2,3,4,8,9,10,11,13-octahydrosilino[3,2-g:5,6-g′]diquinolin-1-ium (019)

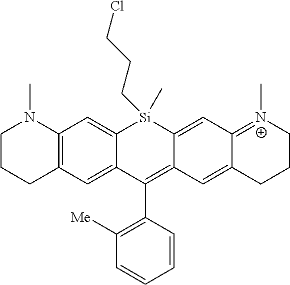 [SMILES: Cc1ccccc1C1=c2cc3c(cc2[Si](C)(CCCCl)c2cc4c(cc21)CCCN4C)=[N+](C)CCC3]

The same procedure was used as described above for compound 016. A solution of 4-1 (0.20 g, 0.36 mmol) in anhydrous THF (10.0 mL) was treated with s-BuLi (1.4M in cyclohexane) (0.57 mL, 0.79 mmol) and dichloro(3-chloropropyl)(methyl)silane (73.0 μL, 0.47 mmol). The resulting residue was re-dissolved in DCM (20.0 mL), followed by treatment with p-chloranil (0.18 g, 0.72 mmol) to provide 019 as an inseparable mixture of diastereomers (48.0 mg, 26%) in green color solid. ¹H NMR (500 MHz, CDCl₃) δ 7.46-7.40 (m, 1H), 7.37-7.30 (m, 2H), 7.06 (d, J=8.0 Hz, 1H), 7.04 (s, 2H), 6.65 (s, 2H), 3.59 (t, J=5.5 Hz, 4H), 3.51-3.41 (m, 2H), 3.34 (s, 6H), 2.51 (t, J=6.0 Hz, 4H), 2.03 and 2.02 (2×s, 3H), 1.99-1.88 (m, 4H), 1.78-1.64 (m, 2H), 1.21-1.10 (m, 2H), 0.62 and 0.60 (2×s, 3H) ppm; ¹³C NMR (125 MHz, CDCl₃) δ 168.54 (2 signals), 152.04 (2 signals), 146.01 (2 signals), 138.79 (2 signals), 138.33 (2 signals), 135.80, 135.57, 130.38 (2 signals), 129.23, 128.96, 128.94, 128.91, 128.32, 128.22, 125.78 (2 signals), 124.83, 119.91 (2 signals), 52.79, 47.57 (2 signals), 40.07, 27.51, 27.07 (2 signals), 20.96, 19.67, 13.87 (2 signals), −3.09 (2 signals) ppm; ¹⁹F NMR (470 MHz, CDCl₃); −75.68 ppm; HRMS (ESI) m/z: [M]⁺ calcd for C₃₂H₃₈ClN₂Si, 513.2487; found 513.2483.

### Example 25: 1,9,11,11-Tetramethyl-5-(o-tolyl)-2,3,7,8,9,11-hexahydrosilino[3,2-f:5,6-f]diindol-1-ium (020)

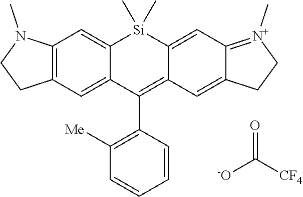

The same procedure was used as described above for compound 016. A solution of 5 (0.20 g, 0.38 mmol) in anhydrous THE (10.0 mL) was treated with s-BuLi (1.4M in cyclohexane) (0.60 mL, 0.84 mmol) and dichlorodimethyllsilane (60.0 μL, 0.50 mmol). The resulting residue was redissolved in DCM (20.0 mL), followed by treatment with p-chloranil (0.19 g, 0.76 mmol) to provide 020 (60.0 mg, 37%) as a green solid. ¹H NMR (500 MHz, CDCl₃) δ 7.46-7.41 (m, 1H), 7.38-7.31 (m, 2H), 7.06 (d, J=7.5 Hz, 1H), 6.88 (s, 2H), 6.67 (s, 2H), 3.82 (t, J=7.0 Hz, 4H), 3.21 (s, 6H), 2.95 (t, J=7.5 Hz, 4H), 2.03 (s, 3H), 0.56 (s, 3H), 0.53 (s, 3H) ppm; ¹³C NMR (125 MHz, CDCl₃) δ 166.99, 157.08, 151.09, 139.58, 135.70, 133.22, 132.95, 130.49, 128.96, 128.90, 128.79, 125.97, 114.44, 54.80, 33.87, 26.57, 19.50, −0.94, −1.36 ppm; ¹⁹F NMR (470 MHz, CDCl₃); −75.85 ppm; HRMS (ESI) m/z: [M]⁺ calcd for C₂₈H₃₁N₂Si, 423.2251; found 423.2246.

### Example 26: 1,9-Dimethyl-5-(o-tolyl)-11,11-divinyl-2,3,7,8,9,11-hexahydrosilino[3,2-f:5,6-f]diindol-1-ium (021)

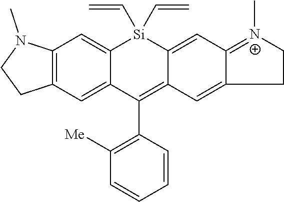 [SMILES: C=C[Si]1(C=C)c2cc3c(cc2C(c2ccccc2C)=c2cc4c(cc21)=[N+](C)CC4)CCN3C]

The same procedure was used as described above for compound 016. A solution of 5-1 (0.20 g, 0.38 mmol) in anhydrous THF (10.0 mL) was treated with s-BuLi (1.4M in cyclohexane) (0.60 mL, 0.84 mmol) and dichlorodivinyllsilane (70.0 μL, 0.50 mmol). The resulting residue was re-dissolved in DCM (20.0 mL), followed by treatment with p-chloranil (0.19 g, 0.76 mmol) to provide 021 (80.0 mg, 47%) as a green solid. ¹H NMR (500 MHz, CDCl₃) δ 7.46-7.41 (m, 1H), 7.38-7.32 (m, 2H), 7.05 (d, J=7.5 Hz, 1H), 6.80 (s, 2H), 6.68 (s, 2H), 6.43-6.24 (m, 4H), 6.02 (dd, J=19.5, 3.0 Hz, 1H), 5.93 (dd, J=19.0, 4.0 Hz, 1H), 3.85 (t, J=8.0 Hz, 4H), 3.20 (s, 6H), 2.96 (t, J=7.5 Hz, 4H), 2.02 (s, 3H) ppm; ¹³C NMR (125 MHz, CDCl₃) δ 166.84, 157.00, 146.48, 139.54, 139.32, 139.06, 135.72, 133.40, 133.30, 131.04, 130.66, 130.54, 129.26, 128.98, 128.87, 125.97, 115.72, 54.83, 33.85, 26.56, 19.46 ppm; ¹⁹F NMR (470 MHz, CDCl₃); −75.85 ppm; IRMS (ESI) m/z: [M]⁺ calcd for C₃₀H₃₁N₂Si, 447.2251; found 447.2246.

### Example 27: 1,9-Dimethyl-11,11-diphenyl-5-(o-tolyl)-2,3,7,8,9,11-hexahydrosilino[3,2-f:5,6-f′]diindol-1-ium (022)

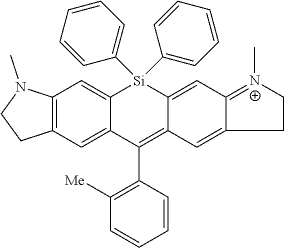 [SMILES: Cc1ccccc1C1=c2cc3c(cc2[Si](c2ccccc2)(c2ccccc2)c2cc4c(cc21)CCN4C)=[N+](C)CC3]

The same procedure was used as described above for compound 016. A solution of 5-1 (0.20 g, 0.38 mmol) in anhydrous THF (10.0 mL) was treated with s-BuLi (1.4M in cyclohexane) (0.60 mL, 0.84 mmol) and dichlorodiphenyllsilane (0.11 mL, 0.50 mmol). The resulting residue was re-dissolved in DCM (20.0 mL), followed by treatment with p-chloranil (0.19 g, 0.76 mmol) to provide 022 (78.0 mg, 38%) as a green solid. ¹H NMR (500 MHz, CDCl₃) δ 7.67-7.62 (m, 2H), 7.61-7.56 (m, 2H), 7.55-7.42 (m, 7H), 7.39-7.33 (m, 2H), 7.09 (d, J=7.0 Hz, 1H), 6.79 (s, 2H), 6.73 (s, 2H), 3.85 (t, J=7.5 Hz, 4H), 3.10 (s, 6H), 2.97 (t, J=7.5 Hz, 4H), 2.02 (s, 3H) ppm; ¹³C NMR (125 MHz, CDCl₃) δ 166.87, 156.98, 147.42, 139.28, 136.12, 135.91, 135.70, 133.51, 133.44, 131.48, 131.30, 131.22, 130.91, 130.61, 129.57, 129.02, 128.98, 128.95, 126.03, 116.19, 54.93, 33.94, 26.59, 19.49 ppm; ¹⁹F NMR (470 MHz, CDCl₃); −75.85 ppm; HRMS (ESI) m/z: [M]⁺ calcd for C₃₈H₃₅N₂Si, 547.2564; found 547.2557.

### Example 28: 11-(3-Chloropropyl)-1,9,11-trimethyl-5-(o-tolyl)-2,3,7,8,9,11-hexahydrosilino[3,2-f:5,6-f]diindol-1-ium (023)

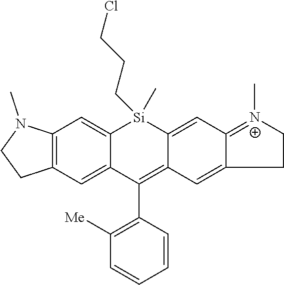 [SMILES: Cc1ccccc1C1=c2cc3c(cc2[Si](C)(CCCCl)c2cc4c(cc21)CCN4C)=[N+](C)CC3]

The same procedure was used as described above for compound 016. A solution of 5-1 (0.20 g, 0.38 mmol) in anhydrous THF (10.0 mL) was treated with s-BuLi (1.4M in cyclohexane) (0.60 mL, 0.84 mmol) and dichloro(3-chloropropyl)(methyl)silane (77.0 μL, 0.50 mmol). The resulting residue was re-dissolved in DCM (20.0 mL), followed by treatment with p-chloranil (0.19 g, 0.76 mmol) to provide 023 as an inseparable mixture of diastereomers (75.0 mg, 41%) in green color solid. ¹H NMR (500 MHz, CDCl₃) δ 7.46-7.40 (m, 1H), 7.38-7.32 (m, 2H), 7.04 (t, J=7.5 Hz, 1H), 6.9 and 6.90 (2×s, 2H), 6.66 and 6.65 (2×s, 2H), 3.83 (t, J=8.0 Hz, 4H), 3.46 and 3.42 (2×t, J=7.0 Hz, 2H), 3.23 (s, 6H), 2.95 (t, J=8.0 Hz, 4H), 2.03 and 2.02 (2×s, 3H), 1.74-1.61 (m, 2H), 1.18-1.10 (m, 2H), 0.63 and 0.61 (2×s, 3H) ppm; ¹³C NMR (125 MHz, CDCl₃) δ 166.91 (2 signals), 157.06 (2 signals), 149.35 (2 signals), 139.55 (2 signals), 135.81, 135.54, 133.21 (2 signals), 133.15, 130.55, 130.46, 129.26, 129.17, 129.12, 128.84, 128.81, 126.01 (2 signals), 114.59 (2 signals), 54.79, 47.37 (2 signals), 33.90, 26.98 (2 signals), 26.56, 19.58 (2 signals), 13.88 (2 signals), −3.59 (2 signals) ppm; ¹⁹F NMR (470 MHz, CDCl₃); −75.81 ppm; HRMS (ESI) m/z: [M]⁺ calcd for C₃₀H₃₄ClN₂Si, 485.2174; found 485.2170.

Rhodamine dyes that can spirolactonize are valuable for live cell imaging, as the spirolactone form is nonfluorescent and cell permeable, whereas the zwitterionic form is brightly fluorescent and can selectively form when bound to particular target biomolecules. Therefore a series of Si-modified Si-rhodamine spirolactones were synthesized (Scheme 4).

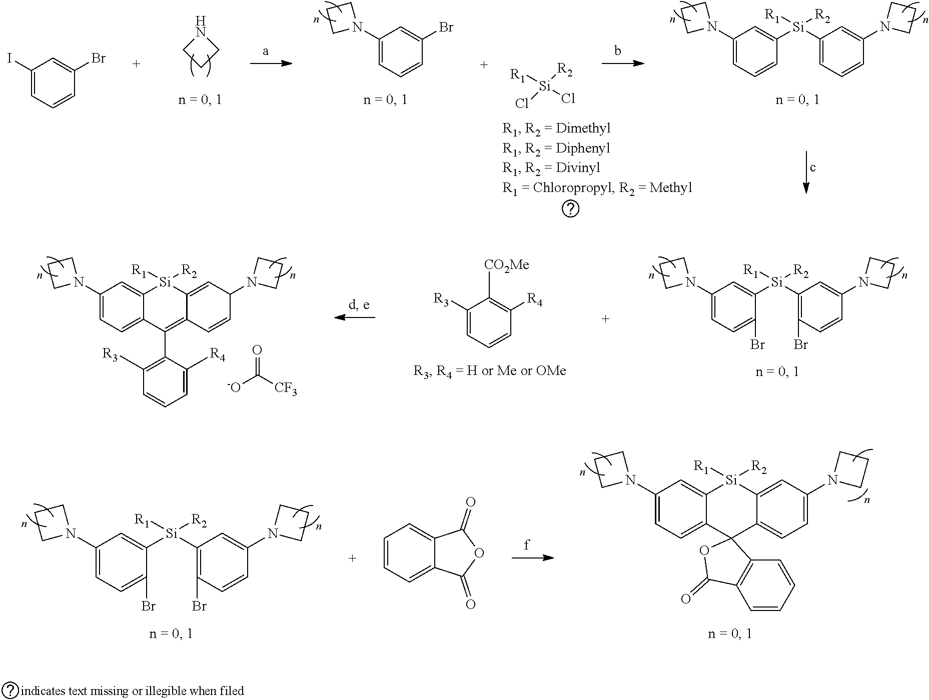

Scheme 4. (a) CuI, K₃PO₄, Ethylene glycol, 1-Butanol, 100° C., 18 h, 78%; (b) n-BuLi (2.5M in hexanes), THF, −78° C. to RT, RT 3 h, 87-95%; (c) NBS, ACN/DCM (2:1), 0° C., 1 h, 63-87%; (d) s-BuLi (1.4M in cyclohexane), THF, −78° C. to −20° C., −20° C. to RT, RT, 18 h; (e) MeOH, AcOH, RT, 10 min, 17-78%, over two steps; (f) s-BuLi (1.4M in cyclohexane), THF, −78° C. to −20° C., −20° C. to RT, RT, 18 h, 12-25%. R¹ and R² in Scheme 4 correspond to R⁶ and R⁷ in Formula (I).

### Example 29:1-(3-Bromophenyl) azetidine (6-1)

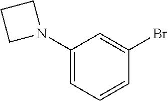 [SMILES: Brc1cccc(N2CCC2)c1]

An oven-dried sealed tube was charged with CuI (0.41 g, 2.12 mmol) and K₃PO₄ (13.6 g, 63.9 mmol). The vial was capped with rubber septum and evacuated/backfilled with argon. Anhydrous 1-butanol (40.0 mL) was added, followed by ethylene glycol (2.89 mL, 51.1 mmol), 3-bromoiodobenzene (2.71 mL, 21.3 mmol), and azetidine (1.72 mL, 25.6 mmol). The vial was sealed with teflon cap under argon and the reaction mixture was stirred at 100° C. for 18 h. The reaction mixture was cooled to room temperature and diluted with saturated NH₄Cl solution (100 mL). After extraction with EtOAc (2×150 mL), the combined extracts were washed with saturated NaCl solution (150 mL), dried (Na₂SO₄), filtered, and evaporated under reduced pressure. The resulting residue was purified by flash column chromatography (Silicycle column, 50 g, gradient elution with 0-10% EtOAc/Hexanes, linear gradient for 20 min) to provide 6-1 (3.50 g, 78%) as a colorless oil. ¹H NMR (500 MHz, CDCl₃) δ 7.04 (t, J=8.0 Hz, 1H), 6.84-6.80 (m, 1H), 6.55 (t, J=2.0 Hz, 1H), 6.33 (dd, J=8.0, 2.0 Hz, 1H), 3.87 (t, J=7.0 Hz, 4H), 2.37 (p, J=7.0 Hz, 2H) ppm; ¹³C NMR (125 MHz, CDCl₃) δ 153.34, 130.30, 123.16, 120.03, 114.20, 109.95, 52.43, 16.99 ppm; HRMS (ESI) m/z: [M+H]⁺ calcd for C₉H₁₁BrN, 212.0069; found 212.0068.

### Example 30: Bis(3-(azetidin-1-yl)phenyl)dimethylsilane (7-1)

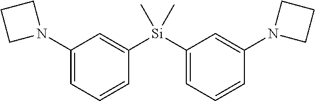 [SMILES: C[Si](C)(c1cccc(N2CCC2)c1)c1cccc(N2CCC2)c1]

A degassed solution of 6-1 (1.20 g, 5.66 mmol) in anhydrous THE (25.0 mL) under argon atmosphere was cooled to −78° C. in an acetone/dry ice bath. After 15 min, n-BuLi (2.5M in hexanes) (2.26 mL, 5.66 mmol) was added dropwise over 10 min. The resulting reaction mixture was stirred at −78° C. for additional 30 min. At the same temperature, dichlorodimethylsilane (0.29 mL, 2.38 mmol) dissolved in anhydrous THE (5.0 mL) was added dropwise over 5 min. The dry ice bath was removed, and reaction mixture was stirred at room temperature for 3 h. It was subsequently quenched with saturated NH₄Cl (25.0 mL), diluted with water (25.0 mL), and then extracted with EtOAc (2×50.0 mL), the combined extracts were washed with saturated NaCl solution (50.0 mL), dried (Na₂SO₄), filtered, and evaporated under reduced pressure. The resulting residue was purified by flash column chromatography (Silicycle column, 50 g, gradient elution with 0-15% EtOAc/Hexanes, linear gradient for 20 min) to provide 7-1 (0.72 g, 94%) as a colorless gummy solid. ¹H NMR (500 MHz, CDCl₃) δ 7.20 (t, J=7.5 Hz, 2H), 6.90 (dt, J=7.0, 1.0 Hz, 2H), 6.61 (d, J=2.5 Hz, 2H), 6.47 (dd, J=8.0, 2.0 Hz, 2H), 3.86 (t, J=7.5 Hz, 8H), 2.34 (p, J=7.0 Hz, 4H), 0.50 (s, 6H) ppm; ¹³C NMR (125 MHz, CDCl₃) δ 151.60, 138.94, 128.36, 123.48, 116.91, 112.31, 52.63, 17.16, −2.10 ppm; HRMS (ESI) m/z: [M+H]⁺ calcd for C₂₀H₂₇N₂Si, 323.1938; found 323.1935.

### Example 31: 3,3′-(Dimethylsilanediyl)bis(N,N-dimethylaniline) (8-1)

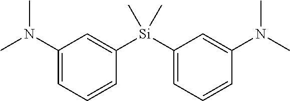

The same procedure was used as described above for compound 7-1. A solution of 3-bromo-N,N-dimethylaniline (2.50 g, 12.5 mmol) in anhydrous THF (30.0 mL) was treated with n-BuLi (2.5M in hexanes) (5.0 mL, 12.5 mmol) and dichlorodimethylsilane (0.63 mL, 5.25 mmol) to provide 8-1 (1.50 g, 95%) as a colorless gummy solid. ¹H NMR (500 MHz, CDCl₃) δ 7.30-7.27 (m, 2H), 6.98 (d, J=2.5 Hz, 2H), 6.95 (d, J=7.5 Hz, 2H), 6.80 (dd, J=8.0, 2.0 Hz, 2H), 2.96 (s, 12H), 0.57 (s, 6H) ppm; ¹³C NMR (125 MHz, CDCl₃) δ 150.06, 139.09, 128.61, 122.87, 118.48, 113.70, 40.83, −2.02 ppm; HRMS (ESI) m/z: [M+H]⁺ calcd for C₁₈H₂₇N₂Si, 299.1938; found 299.1935.

### Example 32: Bis(3-(azetidin-1-yl)phenyl)diphenylsilane (9-1)

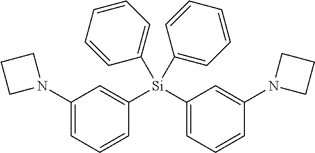 [SMILES: c1ccc([Si](c2ccccc2)(c2cccc(N3CCC3)c2)c2cccc(N3CCC3)c2)cc1]

The same procedure was used as described above for compound 7-1. A solution of 6-1 (1.10 g, 5.19 mmol) in anhydrous THE (25.0 mL) was treated with n-BuLi (2.5M in hexanes) (2.10 mL, 5.19 mmol) and dichlorodiphenylsilane (0.46 mL, 2.18 mmol) to provide 9-1 (0.85 g, 87%) as a colorless gummy solid. ¹H NMR (500 MHz, CDCl₃) δ 7.61-7.56 (m, 4H), 7.42-7.37 (m, 2H), 7.36-7.32 (m, 4H), 7.21 (t, J=7.5 Hz, 2H), 6.92 (d, J=7.0 Hz, 2H), 6.67 (d, J=2.0 Hz, 2H), 6.52 (dd, J=8.0, 2.0 Hz, 2H), 3.80 (t, J=7.5 Hz, 8H), 2.31 (p, J=7.5 Hz, 4H) ppm; ¹³C NMR (125 MHz, CDCl₃) δ 151.59, 136.58, 135.72, 134.94, 134.72, 130.20, 129.46, 128.37, 127.79, 125.77, 119.29, 112.69, 52.58, 17.14 ppm; HRMS (ESI) m/z: [M+H]⁺ calcd for C₃₀H₃₁N₂Si, 447.2251; found 447.2248.

### Example 33: Bis(3-(azetidin-1-yl)phenyl)divinylsilane (10-1)

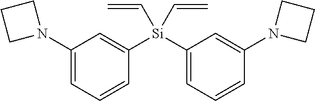 [SMILES: C=C[Si](C=C)(c1cccc(N2CCC2)c1)c1cccc(N2CCC2)c1]

The same procedure was used as described above for compound 7-1. A solution of 6-1 (1.10 g, 5.19 mmol) in anhydrous THE (25.0 mL) was treated with n-BuLi (2.5M in hexanes) (2.10 mL, 5.19 mmol) and dichlorodivinylsilane (0.31 mL, 2.18 mmol) to provide 10-1 (0.70 g, 92%) as a colorless gummy solid. ¹H NMR (500 MHz, CDCl₃) δ 7.21 (t, J=7.5 Hz, 2H), 6.90 (d, J=7.5 Hz, 2H), 6.63 (d, J=2.0 Hz, 2H), 6.49 (dd, J=8.5, 2.0 Hz, 2H), 6.46 (dd, J=20.0, 14.5 Hz, 2H), 6.22 (dd, J=14.5, 3.5 Hz, 2H), 5.81 (dd, J=20.0, 3.5 Hz, 2H), 3.85 (t, J=7.0 Hz, 8H), 2.34 (p, J=7.0 Hz, 4H) ppm; ¹³C NMR (125 MHz, CDCl₃) δ 151.66, 136.11, 134.84, 134.42, 128.38, 124.76, 118.21, 112.65, 52.61, 17.16 ppm; HRMS (ESI) m/z: [M+H]⁺ calcd for C₂₂H₂₇N₂Si, 347.1938; found 347.1932.

### Example 34: 1,1′-(((3-Chloropropyl)(methyl)silanediyl)bis(3,1-phenylene))bis(azetidine) (11-1)

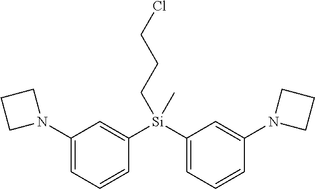 [SMILES: C[Si](CCCCl)(c1cccc(N2CCC2)c1)c1cccc(N2CCC2)c1]

The same procedure was used as described above for compound 7-1. A solution of 6-1 (2.16 g, 10.2 mmol) in anhydrous THE (30.0 mL) was treated with n-BuLi (2.5M in hexanes) (4.07 mL, 10.2 mmol) and dichloro(3-chloropropyl)(methyl)silane (0.67 mL, 4.27 mmol) to provide 11-1 (1.50 g, 91%) as a colorless gummy solid. ¹H NMR (500 MHz, CDCl₃) δ 7.20 (t, J=7.5 Hz, 2H), 6.88 (d, J=7.5 Hz, 2H), 6.60 (d, J=2.0 Hz, 2H), 6.48 (dd, J=8.0, 2.0 Hz, 2H), 3.87 (t, J=7.0 Hz, 8H), 3.50 (t, J=7.0 Hz, 2H), 2.35 (p, J=7.5 Hz, 4H), 1.87-1.79 (m, 2H), 1.16-1.06 (m, 2H), 0.51 (s, 3H) ppm; ¹³C NMR (125 MHz, CDCl₃) δ 151.58, 137.21, 128.46, 123.71, 117.13, 112.52, 52.63, 48.22, 27.78, 17.15, 12.19, −4.17 ppm; HRMS (ESI) m/z: [M+H]⁺ calcd for C₂₂H₃₀ClN₂Si, 385.1861; found 385.1858.

### Example 35: 3,3′-((3-Chloropropyl)(methyl)silanediyl)bis(N,N-dimethylaniline) (12-1)

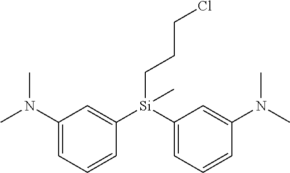

The same procedure was used as described above for compound 7-1. A solution of 3-bromo-N,N-dimethylaniline (5.0 g, 25.0 mmol) in anhydrous THF (50.0 mL) was treated with n-BuLi (2.5M in hexanes) (10.0 mL, 25.0 mmol) and dichloro(3-chloropropyl)(methyl)silane (1.64 mL, 10.5 mmol) to provide 12-1 (3.60 g, 95%) as a colorless gummy solid. ¹H NMR (500 MHz, CDCl₃) δ 7.25 (t, J=8.0 Hz, 2H), 6.93 (d, J=2.0 Hz, 2H), 6.90 (d, J=7.5 Hz, 2H), 6.78 (dd, J=8.0, 2.0 Hz, 2H), 3.52 (t, J=7.0 Hz, 2H), 2.94 (s, 12H), 1.92-1.83 (m, 2H), 1.20-1.13 (m, 2H), 0.55 (s, 3H) ppm; ¹³C NMR (125 MHz, CDCl₃) δ 150.07, 137.36, 128.71, 123.04, 118.64, 113.84, 48.25, 40.80, 27.85, 12.31, −4.09 ppm; HRMS (ESI) m/z: [M+H]⁺ calcd for C₂H₃₀ClN₂Si, 361.1861; found 361.1855.

### Example 36: 3,3′-(Silolane-1,1-diyl)bis(N,N-dimethylaniline) (13-1)

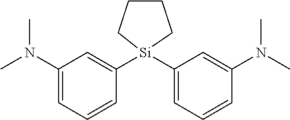

The same procedure was used as described above for compound 7-1. A solution of 3-bromo-N,N-dimethylaniline (2.0 g, 10.0 mmol) in anhydrous THF (30.0 mL) was treated with n-BuLi (2.5M in hexanes) (4.0 mL, 10.0 mmol) and cyclopentyldichlorosilane (0.65 g, 4.19 mmol) to provide 13-1 (1.20 g, 88%) as a colorless gummy solid. ¹H NMR (500 MHz, CDCl₃) δ 7.29-7.23 (m, 2H), 6.97 (d, J=2.5 Hz, 2H), 6.75 (d, J=7.0 Hz, 2H), 6.79 (dd, J=8.5, 3.0 Hz, 2H), 2.94 (s, 12H), 1.81 (p, J=3.5 Hz, 4H), 1.12 (t, J=7.0 Hz, 4H) ppm; ¹³C NMR (125 MHz, CDCl₃) δ 150.06, 137.79, 128.65, 123.37, 118.97, 113.75, 40.79, 27.98, 12.52 ppm; IRMS (ESI) m/z: [M+H]⁺ calcd for C₂₀H₂₉N₂Si, 325.2095; found 325.2089.

### Example 37: Bis(5-(azetidin-1-yl)-2-bromophenyl)dimethylsilane (14-1)

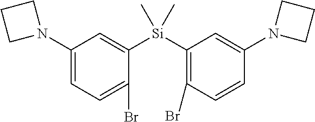 [SMILES: C[Si](C)(c1cc(N2CCC2)ccc1Br)c1cc(N2CCC2)ccc1Br]

A solution of 7-1 (0.70 g, 2.17 mmol) in a mixture of anhydrous ACN/DCM (2:1, 30.0 mL) under argon atmosphere was cooled to 0° C. in an ice-water bath. After 10 min, NBS (0.78 g, 4.38 mmol) was added in small portions over 10 min. The resulting reaction mixture was stirred at 0° C. for 1 h. Saturated NaHCO₃ solution (25.0 mL) was added to the reaction mixture. After extraction with DCM (3×25.0 mL), the combined extracts were washed with saturated NaCl solution (25.0 mL), dried (Na₂SO₄), filtered, and evaporated under reduced pressure. The resulting residue was purified by flash column chromatography (Silicycle column, 25 g, gradient elution with 0-15% EtOAc/Hexanes) to provide 14-1 (0.65 g, 63%) as a white solid. ¹H NMR (500 MHz, CDCl₃) δ 7.31 (d, J=8.5 Hz, 2H), 6.51 (d, J=2.5 Hz, 2H), 6.31 (dd, J=8.5, 3.0 Hz, 2H), 3.81 (t, J=7.5 Hz, 8H), 2.33 (p, J=7.5 Hz, 4H), 0.71 (s, 6H) ppm; ¹³C NMR (125 MHz, CDCl₃) δ 150.59, 138.93, 132.95, 120.41, 117.55, 114.16, 52.59, 17.03, −0.90 ppm; IRMS (ESI) m/z: [M+H]⁺ calcd for C₂₀H₂₅Br₂N₂Si, 479.0148; found 479.0150.

### Example 38: 3,3′-(Dimethylsilanediyl)bis(4-bromo-N,N-dimethylaniline) (15-1)

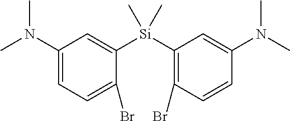

The same procedure was used as described above for compound 14-1. A solution of 8-1 (1.50 g, 5.02 mmol) in a mixture of anhydrous ACN/DCM (2:1, 46.0 mL) was treated with NBS (1.81 g, 10.2 mmol) to provide 15-1 (1.90 g, 83%) as a white solid. ¹H NMR (500 MHz, CDCl₃) δ 7.35 (d, J=8.5 Hz, 2H), 6.84 (d, J=3.5 Hz, 2H), 6.60 (dd, J=8.5, 3.0 Hz, 2H), 2.88 (s, 12H), 0.76 (s, 6H) ppm; ¹³C NMR (125 MHz, CDCl₃) δ 149.04, 138.88, 133.11, 121.93, 116.94, 115.40, 40.71, −0.79 ppm; HRMS (ESI) m/z: [M+H]⁺ calcd for C₁₈H₂₅Br₂N₂Si, 455.0148; found 455.0151.

### Example 39: Bis(5-(azetidin-1-yl)-2-bromophenyl)diphenylsilane (16-1)

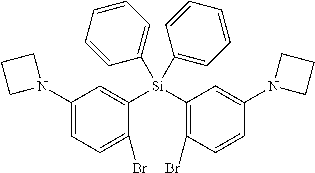 [SMILES: Brc1ccc(N2CCC2)cc1[Si](c1ccccc1)(c1ccccc1)c1cc(N2CCC2)ccc1Br]

The same procedure was used as described above for compound 14-1. A solution of 9-1 (0.78 g, 1.75 mmol) in a mixture of anhydrous ACN/DCM (2:1, 30.0 mL) was treated with NBS (0.64 g, 3.58 mmol) to provide 16-1 (0.80 g, 76%) as a white solid. ¹H NMR (500 MHz, CDCl₃) δ 7.67-7.61 (m, 4H), 7.42-7.31 (m, 8H), 6.53 (d, J=3.0 Hz, 2H), 6.36 (dd, J=8.5, 3.0 Hz, 2H), 3.71 (t, J=7.5 Hz, 8H), 2.27 (p, J=7.0 Hz, 4H) ppm; ¹³C NMR (125 MHz, CDCl₃) δ 150.47, 136.86, 135.81, 133.83, 133.41, 129.40, 127.65, 123.34, 118.34, 114.50, 52.35, 16.96 ppm; HRMS (ESI) m/z: [M+H]⁺ calcd for C₃₀H₂₉Br₂N₂Si, 603.0461; found 603.0460.

### Example 40: Bis(5-(azetidin-1-yl)-2-bromophenyl)divinylsilane (17-1)

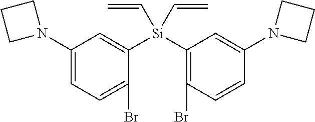 [SMILES: C=C[Si](C=C)(c1cc(N2CCC2)ccc1Br)c1cc(N2CCC2)ccc1Br]

The same procedure was used as described above for compound 14-1. A solution of 10-1 (0.67 g, 1.93 mmol) in a mixture of anhydrous ACN/DCM (2:1, 24.0 mL) was treated with NBS (0.71 g, 3.96 mmol) to provide 17-1 (0.75 g, 77%) as a white solid. ¹H NMR (500 MHz, CDCl₃) δ 7.31 (d, J=8.5 Hz, 2H), 6.66 (dd, J=20.0, 14.5 Hz, 2H), 6.61 (d, J=3.0 Hz, 2H), 6.33 (dd, J=8.5, 3.0 Hz, 2H), 6.26 (dd, J=14.5, 3.5 Hz, 2H), 5.84 (dd, J=20.5, 3.5 Hz, 2H), 3.82 (t, J=7.0 Hz, 8H), 2.33 (p, J=7.5 Hz, 4H) ppm; ¹³C NMR (125 MHz, CDCl₃) δ 150.59, 136.40, 136.25, 133.51, 132.97, 121.62, 117.58, 114.44, 52.57, 17.02 ppm; HRMS (ESI) m/z: [M+H]⁺ calcd for C₂₂H₂₅Br₂N₂Si, 503.0148; found 503.0151.

### Example 41: 1,1′-(((3-Chloropropyl)(methyl)silanediyl)bis(4-bromo-3,1-phenylene))bis(azetidine) (18-1)

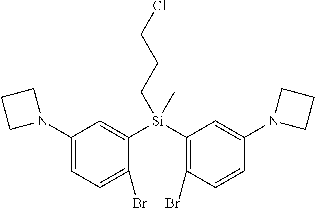 [SMILES: C[Si](CCCCl)(c1cc(N2CCC2)ccc1Br)c1cc(N2CCC2)ccc1Br]

The same procedure was used as described above for compound 14-1. A solution of 11-1 (1.0 g, 2.60 mmol) in a mixture of anhydrous ACN/DCM (2:1, 30.0 mL) was treated with NBS (0.94 g, 5.25 mmol) to provide 18-1 (1.16 g, 82%) as a white gummy solid. ¹H NMR (500 MHz, CDCl₃) δ 7.31 (d, J=8.5 Hz, 2H), 6.49 (d, J=2.5 Hz, 2H), 6.33 (dd, J=8.5, 2.0 Hz, 2H), 3.82 (t, J=7.5 Hz, 8H), 3.54 (t, J=7.0 Hz, 2H), 2.34 (p, J=7.5 Hz, 4H), 1.83-1.74 (m, 2H), 1.46-1.40 (m, 2H), 0.71 (s, 3H) ppm; ¹³C NMR (125 MHz, CDCl₃) δ 150.53, 137.58, 133.04, 120.62, 117.48, 114.32, 52.60, 48.26, 27.98, 17.01, 12.05, −2.64 ppm; HRMS (ESI) m/z: [M+H]⁺ calcd for C₂₂H₂₈Br₂ClN₂Si, 541.0072; found 541.0076.

### Example 42: 3,3′-((3-Chloropropyl)(methyl)silanediyl)bis(4-bromo-N,N-dimethylaniline) (19-1)

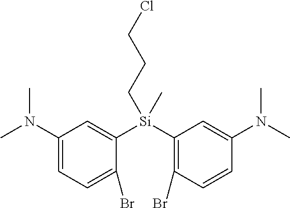

The same procedure was used as described above for compound 14-1. A solution of 12-1 (3.60 g, 9.97 mmol) in a mixture of anhydrous ACN/DCM (2:1, 60.0 mL) was treated with NBS (3.58 g, 20.2 mmol) to provide 19-1 (4.50 g, 87%) as a white gummy solid. ¹H NMR (500 MHz, CDCl₃) δ 7.35 (d, J=9.0 Hz, 2H), 6.80 (d, J=2.5 Hz, 2H), 6.60 (dd, J=7.0, 2.0 Hz, 2H), 3.55 (t, J=6.5 Hz, 2H), 2.88 (s, 12H), 1.86-1.78 (m, 2H), 1.51-1.45 (m, 2H), 0.75 (s, 3H) ppm; ¹³C NMR (125 MHz, CDCl₃) δ 149.05, 137.53, 133.21, 122.04, 116.78, 115.48, 48.28, 40.70, 28.09, 12.26, −2.54 ppm; IRMS (ESI) m/z: [M+H]⁺ calcd for C₂₀H₂₈Br₂ClN₂Si, 519.0051; found 519.0048.

### Example 43: 3,3′-(Silolane-1,1-diyl)bis(4-bromo-N,N-dimethylaniline) (20-1)

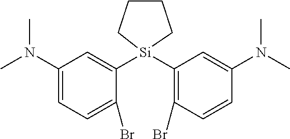

The same procedure was used as described above for compound 14-1. A solution of 13-1 (0.50 g, 1.54 mmol) in a mixture of anhydrous ACN/DCM (2:1, 21.0 mL) was treated with NBS (0.56 g, 3.11 mmol) to provide 20-1 (0.60 g, 81%) as a white solid. ¹H NMR (500 MHz, CDCl₃) δ 7.32 (d, J=8.5 Hz, 2H), 7.02 (d, J=3.0 Hz, 2H), 6.59 (dd, J=8.5, 3.0 Hz, 2H), 2.89 (s, 12H), 1.80 (p, J=3.5 Hz, 4H), 1.34 (t, J=7.0 Hz, 4H) ppm; ¹³C NMR (125 MHz, CDCl₃) δ 148.95, 137.76, 132.84, 122.33, 117.46, 115.54, 40.78, 27.21, 12.82 ppm; HRMS (ESI) m/z: [M+H]⁺ calcd for C₂₀H₂₇Br₂N₂Si, 481.0305; found 481.0308.

### Example 44: 1-(7-(Azetidin-l-yl)-5,5-dimethyl-10-(o-tolyl)dibenzo[b,e]silin-3(5H)-ylidene)azetidin-1-ium (024)

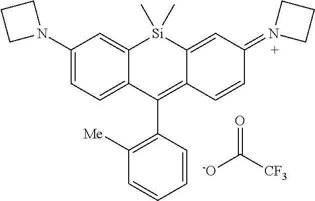

A degassed solution of 14-1 (0.30 g, 0.63 mmol) in anhydrous THE (15.0 mL) under argon atmosphere was cooled to −78° C. in an acetone/dry ice bath. After 15 min, s-BuLi (1.4M in cyclohexane) (1.78 mL, 2.50 mmol) was added dropwise over 5 min. The resulting reaction mixture was stirred at −78° C. for additional 30 min. It was then warmed to −20° C., and a solution of methyl 2-methylbenzoate (0.19 mL, 1.37 mmol) in THE (10.0 mL) was added dropwise over 30 min. The reaction was allowed to warm to room temperature and stirred for overnight (18 h). It was subsequently quenched with saturated NH₄Cl (25.0 mL), diluted with water (25.0 mL), and then extracted with EtOAc (2×50.0 mL), the combined extracts were washed with saturated NaCl solution (25.0 mL), dried (Na₂SO₄), filtered, and evaporated under reduced pressure. The resulting residue was re-dissolved in anhydrous MeOH (15.0 mL), and treated with AcOH (150 μL) at room temperature (immediate dark blue color), and then the mixture solution was stirred for 10 min. The solvent was then evaporated under reduced pressure, and the residue purified by flash column chromatography (Silicycle column, 12 g, 0-5% MeOH in 1% v/v TFA/DCM for 10 min, hold at 5% MeOH isocratic for 5 min, and then increase to 15% gradient over 5 min) to yield (0.11 g, 41%) of the trifluoroacetate salt of 024 as a dark blue solid. ¹H NMR (500 MHz, CDCl₃) δ 7.43-7.39 (m, 1H), 7.35-7.29 (m, 2H), 7.06 (d, J=7.5 Hz, 1H), 7.00 (d, J=9.5 Hz, 2H), 6.75 (d, J=2.5 Hz, 2H), 6.20 (dd, J=9.5, 2.5 Hz, 2H), 4.34 (t, J=7.5 Hz, 8H), 2.59 (p, J=7.5 Hz, 4H), 2.00 (s, 3H), 0.55 (s, 3H), 0.53 (s, 3H) ppm; ¹³C NMR (125 MHz, CDCl₃) δ 169.89, 153.03, 148.28, 141.39, 138.75, 135.78, 130.39, 128.98, 127.59, 125.78, 118.82, 117.09, 114.79, 111.90, 51.99, 19.40, 16.09, −1.01, −1.32 ppm; ¹⁹F NMR (470 MHz, CDCl₃); −75.77 ppm; HRMS (ESI) m/z: [M]⁺ calcd for C₂₈H₃₁N₂Si, 423.2251; found 423.2249.

### Example 45: 1-(7-(Azetidin-1-yl)-5,5-diphenyl-10-(o-tolyl)dibenzo[b,e]silin-3(5H)-ylidene)azetidin-1-ium (025)

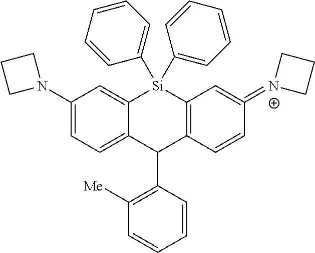

The same procedure was used as described above for compound 024. A solution of 16-1 (0.15 g, 0.25 mmol) in anhydrous THF (10.0 mL) was treated with s-BuLi (1.4M in cyclohexane) (0.71 mL, 0.99 mmol) and methyl 2-methylbenzoate (76 μL, 0.55 mmol) in THE (10 mL). The resulting residue was re-dissolved in MeOH (10.0 mL), followed by treatment with AcOH (100 μL) to provide 025 (50.0 mg, 37%) as a dark blue solid. ¹H NMR (500 MHz, CDCl₃) δ 7.68-7.39 (m, 11H), 7.37-7.30 (m, 2H), 7.13-7.01 (m, 3H), 6.75-6.62 (m, 2H), 6.26 (d, J=8.5 Hz, 2H), 4.26 (t, J=7.5 Hz, 8H), 2.54 (p, J=7.5 Hz, 4H), 2.00 (s, 3H) ppm; ¹³C NMR (125 MHz, CDCl₃) δ 169.83, 152.91, 144.69, 141.67, 138.40, 136.01, 135.85, 131.36, 131.32, 131.25, 130.81, 130.51, 129.15, 129.04, 128.96, 128.94, 128.21, 125.85, 120.70, 112.41, 52.10, 19.41, 16.04 ppm; ¹⁹F NMR (470 MHz, CDCl₃); −75.79 ppm; HRMS (ESI) m/z: [M]⁺ calcd for C₃₈H₃₅N₂Si, 547.2564; found 547.2558.

### Example 46: 1-(7-(Azetidin-1-yl)-10-(o-tolyl)-5,5-divinyldibenzo[b,e]silin-3(5H)-ylidene)azetidin-1-ium (026)

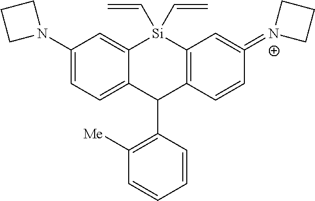

The same procedure was used as described above for compound 024. A solution of 17-1 (0.10 g, 0.20 mmol) in anhydrous THF (5.0 mL) was treated with s-BuLi (1.4M in cyclohexane) (0.36 mL, 0.50 mmol) and methyl 2-methylbenzoate (61.0 μL, 0.44 mmol) in THE (5.0 mL). The resulting residue was re-dissolved in MeOH (5.0 mL), followed by treatment with AcOH (50.0 μL) to provide 026 (15.0 mg, 17%) as a dark blue solid. ¹H NMR (500 MHz, CDCl₃) δ 7.44-7.39 (m, 1H), 7.35-7.29 (m, 2H), 7.06 (d, J=7.5 Hz, 1H), 7.01 (d, J=9.5 Hz, 2H), 6.73-6.65 (m, 2H), 6.43-6.25 (m, 4H), 6.23 (dd, J=9.5, 2.0 Hz, 2H), 5.99 (dd, J=20.0, 3.0 Hz, 1H), 5.91 (dd, J=19.0, 4.0 Hz, 1H), 4.34 (t, J=7.5 Hz, 8H), 2.59 (p, J=7.5 Hz, 4H), 1.99 (s, 3H) ppm; ¹³C NMR (125 MHz, CDCl₃) δ 169.63, 152.94, 143.84, 141.55, 139.52, 139.13, 138.51, 135.83, 130.93, 130.56, 130.44, 129.05, 127.95, 127.31, 125.80, 120.28, 112.20, 52.09, 19.41, 16.10 ppm; ¹⁹F NMR (470 MHz, CDCl₃); −75.62 ppm; HRMS (ESI) m/z: [M]⁺ calcd for C₃₀H₃₁N₂Si, 447.2251; found 447.2250.

### Example 47: 1-(7-(Azetidin-1-yl)-5-(3-chloropropyl)-5-methyl-10-(o-tolyl)dibenzo[b,e]silin-3(5H)-ylidene)azetidin-1-ium (027)

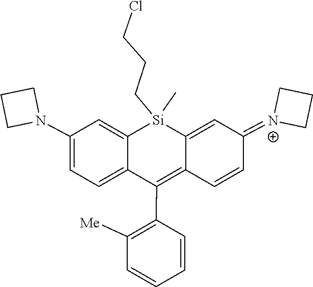 [SMILES: Cc1ccccc1C1=C2C=CC(=[N+]3CCC3)C=C2[Si](C)(CCCCl)c2cc(N3CCC3)ccc21]

The same procedure was used as described above for compound 024. A solution of 18-1 (0.20 g, 0.37 mmol) in anhydrous THF (10.0 mL) was treated with s-BuLi (1.4M in cyclohexane) (0.92 mL, 1.29 mmol) and methyl 2-methylbenzoate (0.12 mL, 0.81 mmol) in THE (10.0 mL). The resulting residue was re-dissolved in MeOH (10.0 mL), followed by treatment with AcOH (100 μL) to provide 027 as an inseparable mixture of diastereomers (65.0 mg, 36%) in dark blue color solid. ¹H NMR (500 MHz, CDCl₃) δ 7.44-7.39 (m, 1H), 7.36-7.29 (m, 2H), 7.04 (d, J=7.5 Hz, 1H), 7.02-6.97 (m, 2H), 6.85-6.80 (m, 2H), 6.21 (dd, J=9.5, 2.0 Hz, 2H), 4.38 (t, J=7.5 Hz, 8H), 3.47 and 3.43 (2×t, J=6.5 Hz, 2H), 2.59 (p, J=7.5 Hz, 4H), 2.01 and 2.00 (2×s, 3H), 1.76-1.61 (m, 2H), 1.20-1.13 (m, 2H), 0.64 and 0.62 (2×s, 3H) ppm; ¹³C NMR (125 MHz, CDCl₃) δ 169.92 (2 signals), 152.96 (2 signals), 146.57 (2 signals), 141.43 (2 signals), 138.67 (2 signals), 135.89, 135.59, 130.43 (2 signals), 129.16, 129.04, 128.82, 127.93, 127.82, 125.84 (2 signals), 119.00 (2 signals), 112.06, 52.07, 47.34 (2 signals), 26.93 (2 signals), 19.42, 16.10, 13.74 (2 signals), −3.57 (2 signals) ppm; ¹⁹F NMR (470 MHz, CDCl₃); −75.79 ppm; HRMS (ESI) m/z: [M]⁺ calcd for C₃₀H₃₄ClN₂Si, 485.2174; found 485.2173.

### Example 48: N-(7-(Dimethylamino)-10-(o-tolyl)-3H-spiro[dibenzo[b,e]siline-5,1′-silolan]-3-ylidene)-N-methylmethanaminium (028)

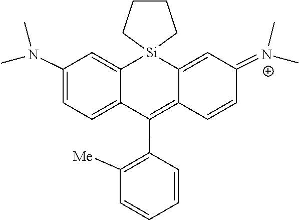

The same procedure was used as described above for compound 024. A solution of 20-1 (0.30 g, 0.62 mmol) in anhydrous THF (15.0 mL) was treated with s-BuLi (1.4M in cyclohexane) (1.95 mL, 2.74 mmol) and methyl 2-methylbenzoate (0.20 mL, 1.37 mmol) in THE (10.0 mL). The resulting residue was re-dissolved in MeOH (15.0 mL), followed by treatment with AcOH (150 μL) to provide 028 (0.11 g, 41%) as a dark blue solid. ¹H NMR (500 MHz, CDCl₃) δ 7.46-7.41 (m, 1H), 7.37-7.31 (m, 2H), 7.12-7.05 (m, 5H), 6.61 (dd, J=10.0, 2.5 Hz, 2H), 3.32 (s, 12H), 2.06-2.00 (m, 4H), 2.03 (s, 3H, overlapping), 1.23-1.14 (m, 4H) ppm; ¹³C NMR (125 MHz, CDCl₃) δ 170.82, 154.16, 148.07, 141.92, 138.40, 135.84, 130.43, 129.17, 128.92, 128.34, 125.82, 120.73, 116.88, 114.59, 114.25, 40.91, 28.81, 28.74, 19.55, 16.11, 15.75 ppm; ¹⁹F NMR (470 MHz, CDCl₃); −75.87 ppm; HRMS (ESI) m/z: [M]⁺ calcd for C₂₈H₃₃N₂Si, 425.2408; found 425.2404.

### Example 49: 1-(7-(Azetidin-1-yl)-5-(3-chloropropyl)-10-(2,6-dimethoxyphenyl)-5-methyldibenzo[b,e]silin-3(5H)-ylidene)azetidin-1-ium (029)

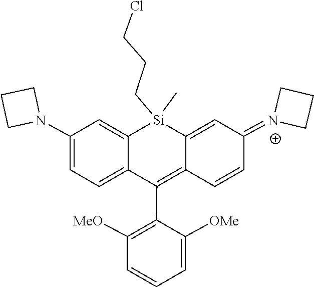 [SMILES: COc1cccc(OC)c1C1=C2C=CC(=[N+]3CCC3)C=C2[Si](C)(CCCCl)c2cc(N3CCC3)ccc21]

The same procedure was used as described above for compound 024. A solution of 18-1 (1.30 g, 2.39 mmol) in anhydrous THF (40.0 mL) was treated with s-BuLi (1.4M in cyclohexane) (6.84 mL, 9.56 mmol) and methyl 2,6-dimethoxybenzoate (1.04 g, 5.27 mmol) in THE (10.0 mL). The resulting residue was re-dissolved in MeOH (50.0 mL), followed by treatment with AcOH (1.0 mL) to provide 029 (1.0 g, 78%) as a dark blue solid. ¹H NMR (500 MHz, CDCl₃) 7.46 (t, J=8.5 Hz, 1H), 7.16 (d, J=9.5 Hz, 2H), 6.71 (d, J=3.0 Hz, 2H), 6.69 (t, J=8.5 Hz, 2H), 6.23 (dd, J=9.0, 2.5 Hz, 2H), 4.33 (t, J=7.5 Hz, 8H), 3.66 (s, 3H), 3.64 (s, 3H), 3.38 (t, J=7.0 Hz, 2H), 2.58 (p, J=8.0 Hz, 4H), 1.69-1.58 (m, 2H), 1.11-1.03 (m, 2H), 0.62 (s, 3H) ppm; ¹³C NMR (125 MHz, CDCl₃) δ 166.82, 157.47, 157.38, 153.11, 146.33, 140.91, 131.10, 128.82, 118.44, 116.07, 112.07, 104.09, 103.86, 56.19, 56.16, 51.99, 47.30, 26.85, 16.15, 14.45, −4.07 ppm; ¹⁹F NMR (470 MHz, CDCl₃); −75.78 ppm; HRMS (ESI) m/z: [M]⁺ calcd for C₃₁H₃₆ClN₂O₂Si, 531.2229; found 531.2226.

### Example 50: N-(5-(3-Chloropropyl)-10-(2,6-dimethoxyphenyl)-7-(dimethylamino)-5-methyldibenzo[b,e]silin-3(5H)-ylidene)-N-methylmethanaminium (030)

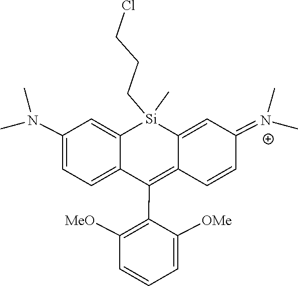

The same procedure was used as described above for compound 024. A solution of 19-1 (1.10 g, 2.12 mmol) in anhydrous THF (40.0 mL) was treated with s-BuLi (1.4M in cyclohexane) (6.06 mL, 8.48 mmol) and methyl 2,6-dimethoxybenzoate (0.92 g, 4.66 mmol) in THE (10.0 mL). The resulting residue was re-dissolved in MeOH (50.0 mL), followed by treatment with AcOH (1.0 mL) to provide 030 (0.80 g, 74%) as a dark blue solid. ¹H NMR (500 MHz, CDCl₃) 7.48 (t, J=8.5 Hz, 1H), 7.25 (d, J=9.5 Hz, 2H), 7.10 (d, J=3.0 Hz, 2H), 6.71 (t, J=8.5 Hz, 2H), 6.62 (dd, J=9.5, 3.0 Hz, 2H), 3.67 (s, 3H), 3.65 (s, 3H), 3.39 (t, J=6.5 Hz, 2H), 3.32 (s, 12H), 1.71-1.62 (m, 2H), 1.15-1.08 (m, 2H), 0.66 (s, 3H) ppm; ¹³C NMR (125 MHz, CDCl₃) δ 167.91, 157.48, 157.40, 154.30, 146.76, 141.40, 131.26, 128.96, 120.24, 116.89, 115.74, 114.58, 114.14, 104.08, 103.89, 56.19, 56.17, 47.23, 40.84, 26.82, 14.42, −3.95 ppm; ¹⁹F NMR (470 MHz, CDCl₃); −75.82 ppm; HRMS (ESI) m/z: [M]⁺ calcd for C₂₉H₃₆ClN₂O₂Si, 507.2229; found 507.2221.

### Example 53: 3,7-Di(azetidin-1-yl)-5,5-dimethyl-3′H,5H-spiro[dibenzo[b,e]siline-10,1′-isobenzofuran]-3′-one (031, JF646)

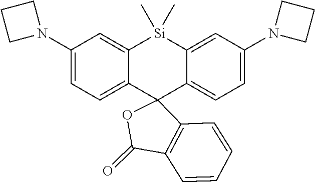 [SMILES: C[Si]1(C)c2cc(N3CCC3)ccc2C2(OC(=O)c3ccccc32)c2ccc(N3CCC3)cc21]

A degassed solution of 14-1 (0.26 g, 0.54 mmol) in anhydrous THE (15.0 mL) under argon atmosphere was cooled to −78° C. in an acetone/dry ice bath. After 15 min, s-BuLi (1.4M in cyclohexane) (1.55 mL, 2.17 mmol) was added dropwise over 5 min. The resulting reaction mixture was stirred at −78° C. for additional 30 min. It was then warmed to −20° C., and a solution of phthalic anhydride (0.18 g, 1.19 mmol) in THE (10.0 mL) was added dropwise over 30 min. The reaction was allowed to warm to room temperature and stirred for overnight (18 h). It was subsequently quenched with saturated NH₄Cl (25.0 mL), diluted with water (25.0 mL), and then extracted with EtOAc (2×50.0 mL), the combined extracts were washed with saturated NaHCO₃ solution (25.0 mL), saturated NaCl solution (25.0 mL), dried (Na₂SO₄), filtered, and evaporated under reduced pressure. The resulting residue was purified by flash column chromatography (Silicycle column, 12 g, 0-25% EtOAc/Hexanes, linear gradient, with constant 20% v/v DCM additive) to provide 031 (60.0 mg, 25%) as a light green solid. ¹H NMR (500 MHz, CDCl₃) δ 7.96 (d, J=7.5 Hz, 1H), 7.64 (td, J=7.5, 1.0 Hz, 1H), 7.54 (td, J=7.5, 0.5 Hz, 1H), 7.31 (d, J=7.5 Hz, 1H), 6.76 (d, J=8.5 Hz, 2H), 6.67 (d, J=2.5 Hz, 2H), 6.25 (dd, J=8.5, 2.5 Hz, 2H), 3.89 (t, J=7.5 Hz, 8H), 2.36 (p, J=7.5 Hz, 4H), 0.61 (s, 3H), 0.59 (s, 3H) ppm; ¹³C NMR (125 MHz, CDCl₃) δ 170.72, 154.31, 151.00, 137.08, 133.71, 132.96, 128.84, 128.02, 127.19, 125.83, 124.83, 115.73, 112.28, 92.09, 52.40, 17.04, 0.53, −1.49 ppm; HRMS (ESI) m/z: [M+H]⁺ calcd for C₂₈H₂₉N₂O₂Si, 453.1993; found 453.1988.

### Example 54: 3,7-Bis(dimethylamino)-5,5-dimethyl-3′H,5H-spiro[dibenzo[b,e]siline-10,1′-isobenzofuran]-3′-one (032, SiR)

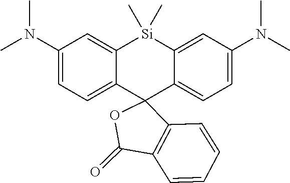

The same procedure was used as described above for compound 031. A solution of 15-1 (0.40 g, 0.88 mmol) in anhydrous THF (20.0 mL) was treated with s-BuLi (1.4M in cyclohexane) (2.50 mL, 3.51 mmol) and phthalic anhydride (0.29 g, 1.93 mmol) in THE (10.0 mL) to provide 032 (90.0 mg, 24%) as a light green solid. ¹H NMR (500 MHz, CDCl₃) δ 7.97 (d, J=7.5 Hz, 1H), 7.64 (td, J=7.5, 1.5 Hz, 1H), 7.54 (td, J=7.5, 1.0 Hz, 1H), 7.30 (d, J=8.0 Hz, 1H), 6.97 (d, J=2.5 Hz, 2H), 6.79 (d, J=8.5 Hz, 2H), 6.55 (dd, J=9.0, 2.5 Hz, 2H), 2.96 (s, 12H), 0.65 (s, 3H), 0.61 (s, 3H) ppm; ¹³C NMR (125 MHz, CDCl₃) δ 170.82, 154.55, 149.41, 137.10, 133.77, 132.06, 128.79, 128.29, 127.16, 125.76, 124.69, 116.72, 113.44, 91.97, 40.41, 0.56, −1.36 ppm; HRMS (ESI) m/z: [M+H]⁺ calcd for C₂₆H₂₉N₂O₂Si, 429.1993; found 429.1989.

### Example 55: 3,7-Di(azetidin-1-yl)-5,5-diphenyl-3′H,5H-spiro[dibenzo[b,e]siline-10,1′-isobenzofuran]-3′-one (033)

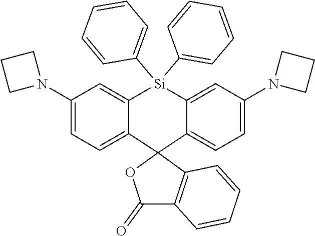 [SMILES: O=C1OC2(c3ccccc31)c1ccc(N3CCC3)cc1[Si](c1ccccc1)(c1ccccc1)c1cc(N3CCC3)ccc12]

The same procedure was used as described above for compound 031. A solution of 16-1 (0.20 g, 0.33 mmol) in anhydrous THF (10.0 mL) was treated with s-BuLi (1.4M in cyclohexane) (0.95 mL, 1.32 mmol) and phthalic anhydride (0.11 g, 0.73 mmol) in THE (10.0 mL) to provide 033 (30.0 mg, 16%) as a light green solid. ¹H NMR (500 MHz, CDCl₃) δ 7.87 (d, J=7.5 Hz, 1H), 7.77-7.72 (m, 2H), 7.59-7.54 (m, 2H), 7.50-7.45 (m, 1H), 7.44-7.39 (m, 3H), 7.37-7.32 (m, 2H), 7.31 (td, J=7.5, 0.5 Hz, 1H), 7.17 (td, J=8.0, 1.0 Hz, 1H), 7.08 (d, J=8.5 Hz, 2H), 6.63 (d, J=2.5 Hz, 2H), 6.61 (d, J=8.0 Hz, 1H), 6.41 (dd, J=8.5, 2.5 Hz, 2H), 3.86-3.75 (m, 8H), 2.30 (p, J=7.0 Hz, 4H) ppm; ¹³C NMR (125 MHz, CDCl₃) δ 171.89, 157.44, 150.75, 136.54, 136.14, 135.07, 134.56, 134.37, 134.04, 130.74, 129.99, 129.96, 128.28, 128.24, 127.98, 127.67, 125.57, 124.17, 123.26, 117.42, 113.74, 90.43, 52.39, 16.96 ppm; HRMS (ESI) m/z: [M+H]⁺ calcd for C₃₈H₃₃N₂O₂Si, 577.2306; found 577.2302.

### Example 56: 3,7-Di(azetidin-1-yl)-5,5-divinyl-3′H,5H-spiro[dibenzo[b,e]siline-10,1′-isobenzofuran]-3′-one (034)

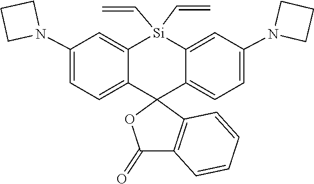 [SMILES: C=C[Si]1(C=C)c2cc(N3CCC3)ccc2C2(OC(=O)c3ccccc32)c2ccc(N3CCC3)cc21]

The same procedure was used as described above for compound 031. A solution of 17-1 (0.25 g, 0.50 mmol) in anhydrous THF (15.0 mL) was treated with s-BuLi (1.4M in cyclohexane) (1.06 mL, 1.49 mmol) and phthalic anhydride (0.17 g, 1.09 mmol) in THE (10.0 mL) to provide 034 (30.0 mg, 12%) as a light green solid. ¹H NMR (500 MHz, CDCl₃) δ 7.91 (d, J=7.5 Hz, 1H), 7.48 (td, J=7.5, 1.0 Hz, 1H), 7.43 (td, J=7.5, 1.0 Hz, 1H), 7.20 (d, J=8.0 Hz, 1H), 6.94 (d, J=8.5 Hz, 2H), 6.63 (d, J=3.0 Hz, 2H), 6.51 (dd, J=20.0, 14.5 Hz, 2H), 6.42-6.26 (m, 4H), 6.03 (dd, J=19.5, 4.0 Hz, 1H), 6.01 (dd, J=20.0, 4.0 Hz, 1H), 3.87 (t, J=7.5 Hz, 8H), 2.35 (p, J=7.5 Hz, 4H) ppm; ¹³C NMR (125 MHz, CDCl₃) δ 171.55, 156.52, 150.80, 137.04, 134.72, 134.31, 134.17, 133.51, 131.18, 128.49, 127.84, 125.71, 125.12, 123.90, 116.87, 113.34, 90.89, 52.41, 17.03 ppm; HRMS (ESI) m/z: [M+H]⁺ calcd for C₃₀H₂₉N₂O₂Si, 477.1993; found 477.1991.

Example 57: 3,7-Di(azetidin-1-yl)-5-(3-chloropropyl)-5-methyl-3′H,5H-spiro[dibenzo[b,e]siline-10,1′-isobenzofuran]-3′-one (035 and 036). The same procedure was used as described above for compound 031. A solution of 18 (0.20 g, 0.37 mmol) in anhydrous THE (10.0 mL) was treated with s-BuLi (1.4M in cyclohexane) (0.80 mL, 1.10 mmol) and phthalic anhydride (0.12 g, 0.81 mmol) in THE (10.0 mL) to provide 035, 036 as a separable mixture of diastereomers (40.0 mg, 21%).

### (5s,10s)-3,7-Di(azetidin-1-yl)-5-(3-chloropropyl)-5-methyl-3′H,5H-spiro[dibenzo[b,e]siline-10,1′-isobenzofuran]-3′-one (035)

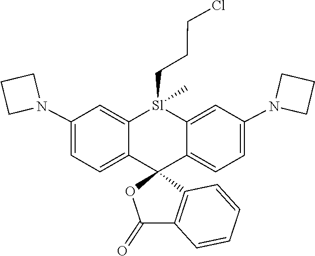

Obtained as a light green color solid. ¹H NMR (500 MHz, CDCl₃) δ 7.96 (d, J=7.5 Hz, 1H), 7.66 (t, J=7.5 Hz, 1H), 7.56 (t, J=7.5 Hz, 1H), 7.31 (d, J=8.0 Hz, 1H), 6.70 (d, J=8.5 Hz, 2H), 6.65 (d, J=2.5 Hz, 2H), 6.26 (dd, J=8.5, 2.5 Hz, 2H), 3.90 (t, J=7.0 Hz, 8H), 3.48 (t, J=7.0 Hz, 2H), 2.37 (p, J=7.5 Hz, 4H), 1.89-1.80 (m, 2H), 1.23-1.16 (m, 2H), 0.62 (s, 3H) ppm; ¹³C NMR (125 MHz, CDCl₃) δ 170.50, 153.99, 150.95, 135.76, 133.75, 133.15, 128.98, 128.41, 127.57, 125.80, 124.98, 115.86, 112.45, 92.18, 52.42, 48.06, 27.74, 17.03, 14.17, −3.64 ppm; HRMS (ESI) m/z: [M+H]⁺ calcd for C₃₀H₃₂ClN₂O₂Si, 515.1916; found 515.1908.

### (5r,10r)-3,7-Di(azetidin-1-yl)-5-(3-chloropropyl)-5-methyl-3′H,5H-spiro[dibenzo[b,e]siline-10,1′-isobenzofuran]-3′-one (036)

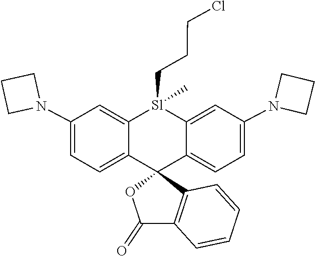

Obtained as an off-white color solid. ¹H NMR (500 MHz, CDCl₃) δ 7.96 (d, J=7.5 Hz, 1H), 7.63 (t, J=7.5 Hz, 1H), 7.54 (t, J=7.5 Hz, 1H), 7.25 (d, J=7.5 Hz, 1H), 6.74 (d, J=9.0 Hz, 2H), 6.64 (d, J=2.5 Hz, 2H), 6.28 (dd, J=8.5, 2.5 Hz, 2H), 3.90 (t, J=7.5 Hz, 8H), 3.54 (t, J=6.5 Hz, 2H), 2.37 (p, J=7.0 Hz, 4H), 1.91-1.81 (m, 2H), 1.30-1.22 (m, 2H), 0.58 (s, 3H) ppm; ¹³C NMR (125 MHz, CDCl₃) δ 170.77, 154.68, 150.92, 134.79, 133.95, 133.12, 128.86, 128.62, 127.08, 125.76, 124.53, 115.45, 112.71, 91.87, 52.40, 48.19, 27.78, 17.04, 12.50, −1.23 ppm; HRMS (ESI) m/z: [M+H]⁺ calcd for C₃₀H₃₂ClN₂O₂Si, 515.1916; found 515.1907.

Example 58: 5-(3-Chloropropyl)-3,7-bis(dimethylamino)-5-methyl-3′H,5H-spiro[dibenzo[b,e]siline-10,1′-isobenzofuran]-3′-one (037 and 038). The same procedure was used as described above for compound 031. A solution of 19 (1.90 g, 3.66 mmol) in anhydrous THE (50.0 mL) was treated with s-BuLi (1.4M in cyclohexane) (10.5 mL, 14.6 mmol) and phthalic anhydride (1.19 g, 8.05 mmol) in THE (10.0 mL) to provide 037, 038 as a separable mixture of diastereomers (0.45 g, 25%).

### (5s,10s)-5-(3-Chloropropyl)-3,7-bis(dimethylamino)-5-methyl-3′H,5H-spiro[dibenzo[b,e]siline-10,1′-isobenzofuran]-3′-one (037)

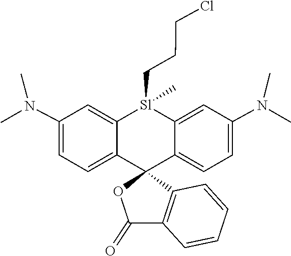

Obtained as a light green color solid. ¹H NMR (500 MHz, CDCl₃) δ 7.97 (dt, J=7.5, 0.5 Hz, 1H), 7.66 (td, J=7.5, 1.5 Hz, 1H), 7.56 (td, J=7.5, 0.5 Hz, 1H), 7.31 (d, J=7.5 Hz, 1H), 6.94 (d, J=3.0 Hz, 2H), 6.73 (d, J=9.0 Hz, 2H), 6.55 (dd, J=9.0, 2.5 Hz, 2H), 3.48 (t, J=6.5 Hz, 2H), 2.97 (s, 12H), 1.91-1.83 (m, 2H), 1.24-1.18 (m, 2H), 0.65 (s, 3H) ppm; ¹³C NMR (125 MHz, CDCl₃) δ 170.62, 154.27, 149.40, 135.76, 133.80, 132.20, 128.92, 128.68, 127.56, 125.72, 124.85, 116.77, 113.52, 92.09, 48.09, 40.39, 27.75, 14.21, −3.50 ppm; HRMS (ESI) m/z: [M+H]⁺ calcd for C₂₈H₃₂ClN₂O₂Si, 491.1916; found 491.1911.

TABLE 2
Crystal data and structure refinement for compound 037.

| Identification code | UMD2365j_a (NM02-54-1) | |
|---|---|---|
| Empirical formula | Empirical formula C28 H31 Cl N2 O2 Si | |
| Formula weight | Formula weight 491.09 | |
| Temperature | Temperature 297(2) K | |
| Wavelength | Wavelength 0.71073 Å | |
| Crystal system | Crystal system Triclinic | |
| Space group | Space group P-1 | |
| Unit cell dimensions | a = 8.2938(5) Å | α = 74.549(2)°. |
| | b = 10.0055(7) Å | β = 88.400(2)°. |
| | c = 17.6368(11) Å | γ = 68.207(2)°. |

| | |
|---|---|
| Volume | 1305.73(15) Å³ |
| Z | 2 |
| Density (calculated) | 1.249 Mg/m³ |
| Absorption coefficient | 0.220 mm⁻¹ |
| F(000) | 520 |
| Crystal size | 0.300 × 0.240 × 0.120 mm³ |
| Crystal color / habit | colorless / plate |
| Theta range for | 2.653 to 26.399°. |
| data collection | |
| Index ranges | −10 <= h <= 10, −12 <= |
| | k <= 12, −22 <= l <= 22 |
| Reflections collected | 49921 |
| Independent reflections | 5289 [R(int) = 0.0284] |
| Completeness to | 98.6% |
| theta = 25.242° | |
| Absorption correction | multi-scan / sadabs |
| Refinement method | Full-matrix least-squares on F2 |
| Data / restraints / parameters | 5289 / 0 / 312 |
| Goodness-of-fit on F² | 1.063 |
| Final R indices | R1 = 0.0404, wR2 = 0.1086 |
| [I > 2sigma(I)] | |
| R indices (all data) | R1 = 0.0487, wR2 = 0.1170 |
| Extinction coefficient | n/a |
| Largest diff. peak and hole | 0.262 and −0.292 e · Å⁻³ |

### (5r,10r)-5-(3-Chloropropyl)-3,7-bis(dimethylamino)-5-methyl-3′H,5H-spiro[dibenzo[b,e]siline-10,1′-isobenzofuran]-3′-one (038)

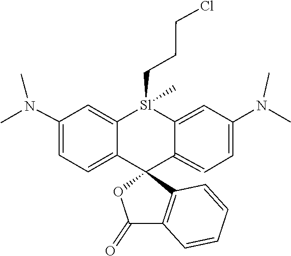

Obtained as a light green color solid. ¹H NMR (500 MHz, CDCl₃) δ 7.97 (dt, J=8.0, 1.0 Hz, 1H), 7.63 (td, J=7.5, 1.5 Hz, 1H), 7.54 (td, J=7.5, 1.0 Hz, 1H), 7.24 (d, J=8.0 Hz, 1H), 6.93 (d, J=2.5 Hz, 2H), 6.77 (d, J=9.0 Hz, 2H), 6.57 (dd, J=9.0, 3.0 Hz, 2H), 3.55 (t, J=6.5 Hz, 2H), 2.97 (s, 12H), 1.94-1.86 (m, 2H), 1.33-1.26 (m, 2H), 0.60 (s, 3H) ppm; ¹³C NMR (125 MHz, CDCl₃) δ 170.86, 154.87, 149.36, 134.87, 133.98, 132.18, 128.51, 128.13, 127.10, 125.70, 124.44, 116.39, 113.78, 91.80, 48.22, 40.40, 27.86, 12.62, −1.18 ppm; HRMS (ESI) m/z: [M+H]⁺ calcd for C₂₈H₃₂ClN₂O₂Si, 491.1916; found 491.1911.

### Example 59: (5r,10r)-3,7-Di(azetidin-1-yl)-5-(3-iodopropyl)-5-methyl-3′H,5H-spiro[dibenzo[b,e]siline-10,1′-isobenzofuran]-3′-one (070)

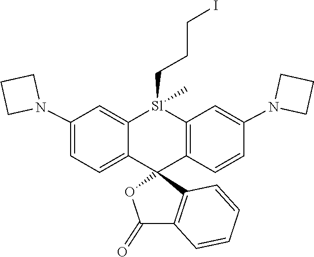

The same procedure was used as described above for compound 0047. A solution of 036 (50.0 mg, 0.097 mmol) in anhydrous acetone (5.0 mL) was treated with NaI (58.0 mg, 0.39 mmol) to provide 070 (48.0 mg, 81%) as a light green color solid. ¹H NMR (500 MHz, CDCl₃) δ 7.96 (d, J=8.0 Hz, 1H), 7.64 (td, J=7.5, 1.0 Hz, 1H), 7.54 (td, J=8.0, 0.5 Hz, 1H), 7.25 (d, J=8.0 Hz, 1H), 6.74 (d, J=8.5 Hz, 2H), 6.63 (d, J=2.0 Hz, 2H), 6.28 (dd, J=8.5, 2.5 Hz, 2H), 3.90 (t, J=7.0 Hz, 8H), 3.24 (t, J=6.5 Hz, 2H), 2.37 (p, J=7.0 Hz, 4H), 1.92-1.83 (m, 2H), 1.28-1.21 (m, 2H), 0.58 (s, 3H) ppm; ¹³C NMR (125 MHz, CDCl₃) δ 170.80, 154.70, 150.91, 134.76, 133.99, 133.08, 128.86, 128.63, 127.04, 125.76, 124.53, 115.43, 112.74, 91.86, 52.43, 28.72, 17.04, 16.65, 12.14, −1.19 ppm; HRMS (ESI) m/z: [M+H]⁺ calcd for C₃₀H₃₂IN₂O₂Si, 607.1272; found 607.1272.

### Example 60: 2-((3-((5r,10r)-3,7-Di(azetidin-1-yl)-5-methyl-3′-oxo-3′H,5H-spiro[dibenzo[b,e]siline-10,1′-isobenzofuran]-5-yl)propyl)thio)acetic acid (069)

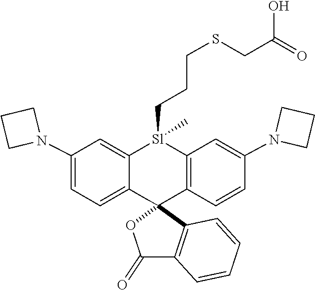

The same procedure was used as described above for compound 063. A solution of 070 (32.0 mg, 0.054 mmol) in a mixture of anhydrous MeOH/THF (1:1, 2.0 mL) was treated with 1M NaOH (0.11 mL, 0.11 mmol) to provide crude acid 069 (30.0 mg, 98%) as a blue color solid. HRMS (ESI) m/z: [M+H]⁺ calcd for C₃₂H₃₅N₂O₄SSi, 571.2082; found 571.2082.

### Example 61: N-(2-(2-((6-Chlorohexyl)oxy)ethoxy)ethyl)-2-((3-((5r,10r)-3,7-di(azetidin-1-yl)-5-methyl-3′-oxo-3′H,5H-spiro[dibenzo[b,e]siline-10,1′-isobenzofuran]-5-yl)propyl)thio)acetamide (068)

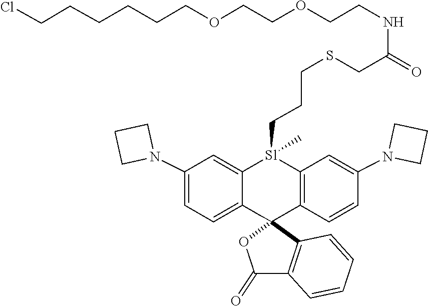

The same procedure was used as described above for compound 050. A solution of 069 (32.0 mg, 0.056 mmol) in anhydrous DMF (1.0 mL) was combined with HaloTag amine (O2) ligand (29.0 mg, 0.084 mmol), treated with HATU (37.0 mg, 0.095 mmol) and DIPEA (50.0 μL, 0.28 mmol) to provide 068 (30.0 mg, 69%) as a light green color solid. ¹H NMR (500 MHz, CDCl₃) δ 7.96 (d, J=7.5 Hz, 1H), 7.63 (t, J=7.5 Hz, 1H), 7.53 (t, J=7.0 Hz, 1H), 7.23 (d, J=8.0 Hz, 1H), 7.12 (t, J=5.5 Hz, 1H), 6.74 (d, J=8.5 Hz, 2H), 6.61 (d, J=2.5 Hz, 2H), 6.27 (dd, J=8.5, 2.5 Hz, 2H), 3.90 (t, J=7.0 Hz, 8H), 3.59-3.50 (m, 8H), 3.48-3.42 (m, 4H), 3.17 (s, 2H), 2.58 (t, J=7.0 Hz, 2H), 2.37 (t, J=7.0 Hz, 4H), 1.80-1.73 (m, 2H), 1.72-1.65 (m, 2H), 1.62-1.55 (m, 2H), 1.49-1.41 (m, 2H), 1.39-1.33 (m, 2H), 1.23-1.18 (m, 2H), 0.56 (s, 3H) ppm; ¹³C NMR (125 MHz, CDCl₃) δ 170.80, 168.96, 150.95, 134.95, 133.99, 132.92, 128.82, 128.74, 126.95, 125.81, 124.51, 115.40, 112.71, 92.00, 71.43, 70.58, 70.17, 69.85, 52.38, 45.18, 39.58, 38.75, 36.58, 36.19, 32.65, 29.61, 26.82, 25.56, 24.08, 17.04, 14.56, −1.33 ppm; IRMS (ESI) m/z: [M+h]⁺ calcd for C₄₂H₅₅ClN₃O₅SSi, 776.3315; found 776.3315.

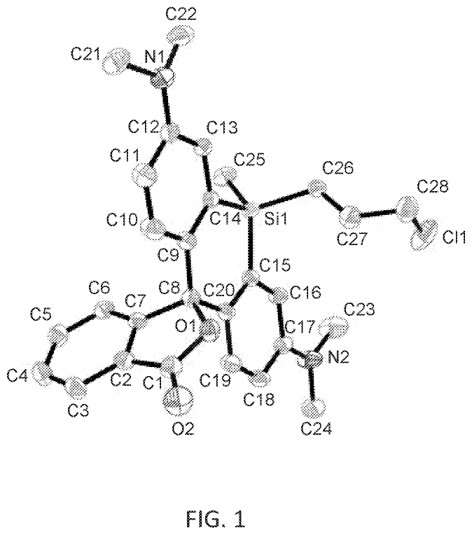

Small molecule X-ray crystallography of compound 037 revealed that it was the “s,s” isomer, where the chloropropyl group and the phenyl of the spirolactone are on opposite faces of the planar dye (FIG. 1).

Displacement of the chloro group in compound 037 and 038 with iodide afforded the corresponding isomeric iodopropylsilyl dyes compounds 047 and 048, which can be further elaborated by reaction with a wide variety of nucleophiles (Scheme 5A). The ability to modify the iodopropyl dyes to incorporate clickable azide groups, amine-reactive NHS esters, and HaloTag® linkers for protein labeling were explored (Scheme 5A). Azide-functionalized dyes were readily synthesized, and reacted rapidly with the strained alkyne DBCO in copper-free click chemistry reactions. Displacement of the iodide with thiols enabled the introduction of an NHS ester, or a HaloTag® chloroalkane ligand. These reagents all hold considerable potential for labeling of biomolecules with bright, photostable near-IR dyes, and it was anticipated that Si-iodopropylsilyl dyes will be easily elaborated with many other nucleophiles, such as amines, phenols, phosphines, and phosphites.

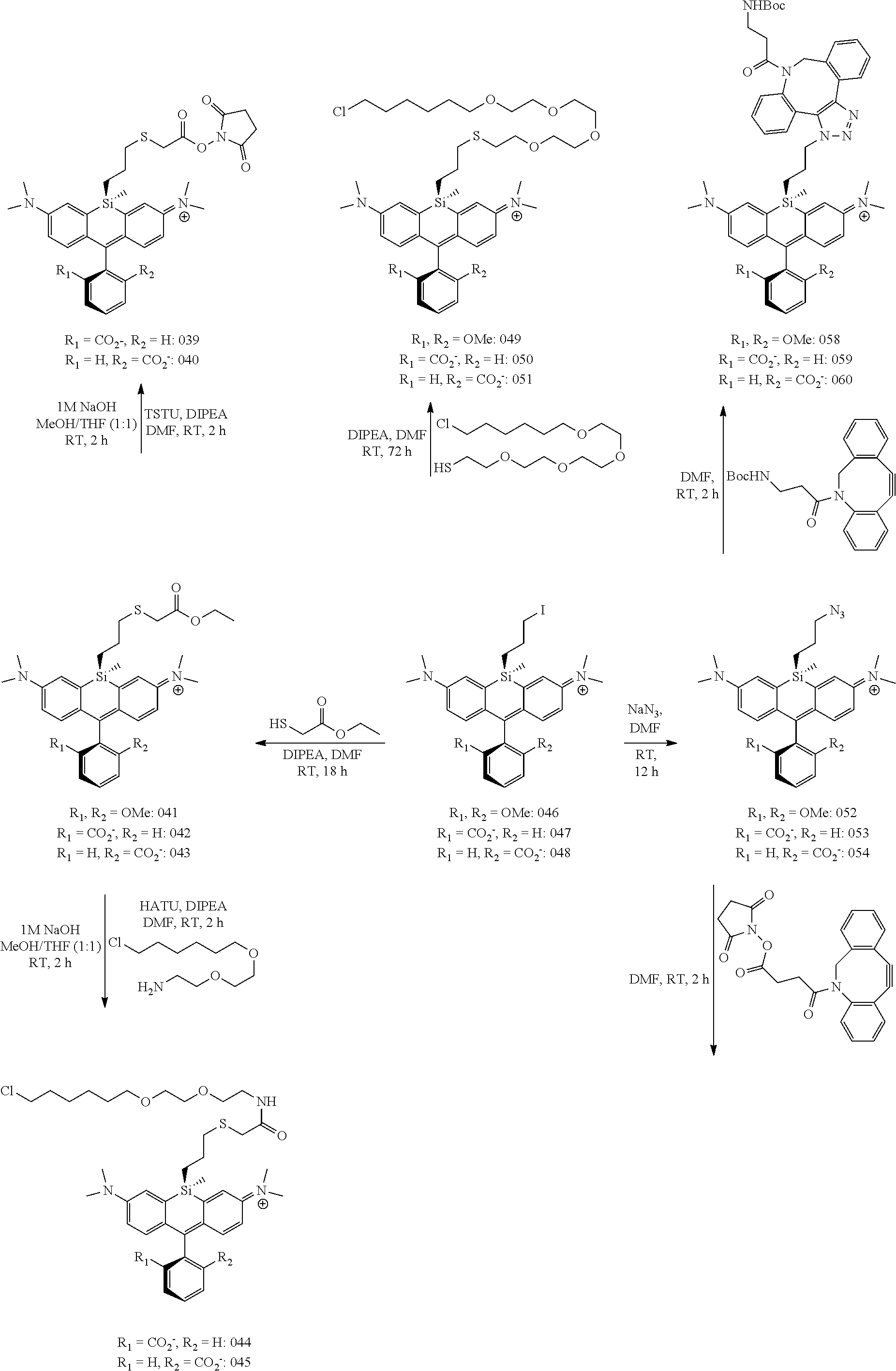

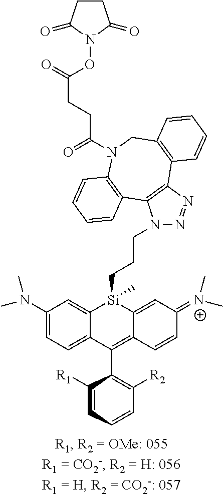

Scheme 5A. Iodopropyl Si-Bridge dyes can be readily elaborated into functionalized dyes with clickable azides, HaloTag® ligands, and amine-reactive NHS esters. All compounds drawn in the ring-opened dye form for simplicity.

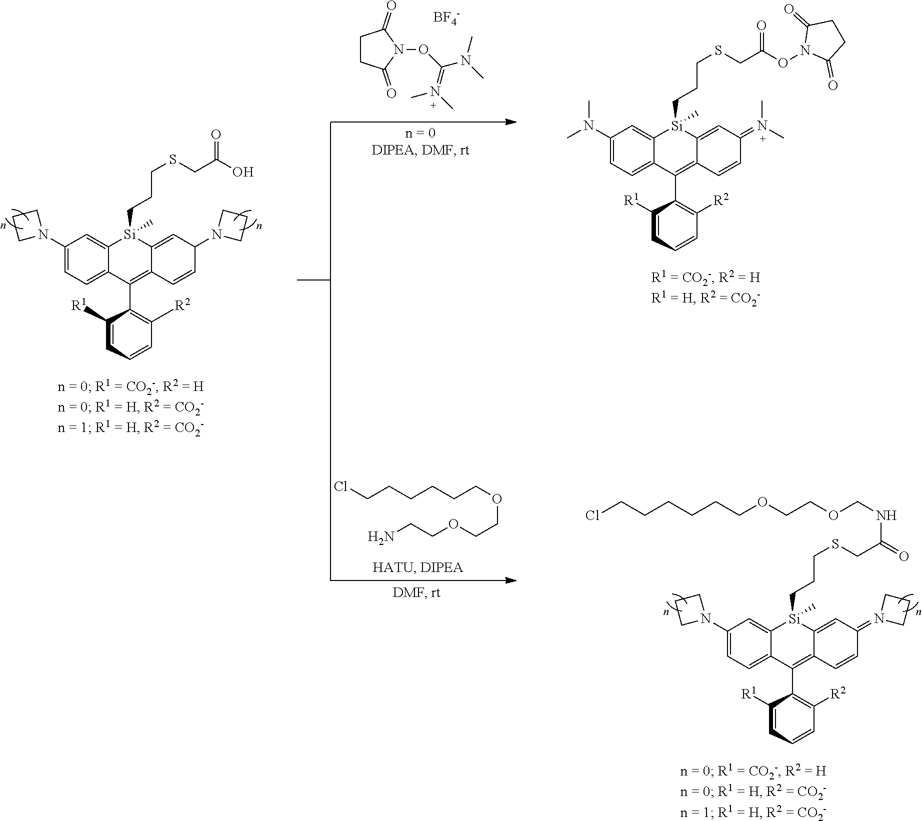

Scheme 5B. Si-bridge NHS esters 039 and 040 and amine (02) HaloTag ligand dyes 044, 045, and 068.

### Example 62: N-(7-(Dimethylamino)-5-(3-iodopropyl)-5-methyl-10-(o-tolyl) dibenzo[b,e]silin-3 (5H)-ylidene)-N-methylmethanaminium (067)

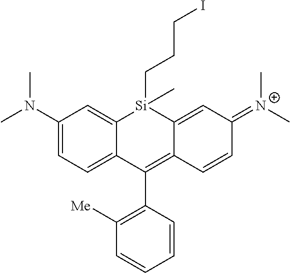

A solution of 009 (0.29 g, 0.63 mmol) in anhydrous acetone (10.0 mL) under argon atmosphere was treated with NaI (0.38 g, 2.51 mmol) at room temperature and reaction mixture was stirred at 80° C. for 18 h. After completion of the reaction, cooled to room temperature and solvent was evaporated under reduced pressure. The resulting residue was purified by flash column chromatography (Silicycle column, 25 g, gradient elution with 0-10% MeOH in 1% v/v TFA/DCM, linear gradient for 20 min) to provide 067 as an inseparable mixture of diastereomers (0.28 g, 81%) in dark blue color solid. 1H NMR (500 MHz, CDCl₃) δ 7.46-7.41 (m, 1H), 7.37-7.31 (m, 2H), 7.16 (t, J=3.0 Hz, 2H), 7.10 (d, J=3.5 Hz, 1H), 7.09-7.06 (m, 2H), 6.61 (dd, J=9.5, 2.5 Hz, 2H), 3.34 (s, 12H), 3.14 and 3.09 (2×t, J=7.0 Hz, 2H), 2.04 and 2.01 (2×s, 3H), 1.80-1.66 (m, 2H), 1.21-1.13 (m, 2H), 0.66 and 0.64 (2×s, 3H) ppm; ¹³C NMR (125 MHz, CDCl₃) δ 170.69 (2 signals), 154.24 (2 signals), 147.00 (2 signals), 141.87 (2 signals), 138.36 (2 signals), 135.88, 135.60, 130.45 (2 signals), 129.17, 129.15, 128.84, 128.09, 127.99, 125.83 (2 signals), 120.93 (2 signals), 119.20, 116.91, 114.61, 114.23, 112.32, 41.01, 28.07 (2 signals), 19.56 (2 signals), 17.80 (2 signals), 10.57 (2 signals), −3.35 (2 signals) ppm; ¹⁹F NMR (470 MHz, CDCl₃); −75.80 ppm; HRMS (ESI) m/z: [M]⁺ calcd for C₂₈H₃₄IN₂Si, 553.1530; found 553.1521.

### Example 63: N-(10-(2,6-Dimethoxyphenyl)-7-(dimethylamino)-5-(3-iodopropyl)-5-methyldibenzo[b,e]silin-3(5H)-ylidene)-N-methylmethanaminium (046)

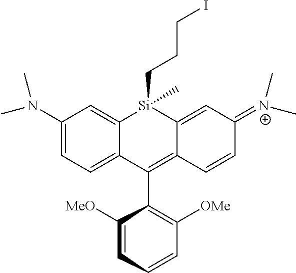

The same procedure was used as described above in Example 59. A solution of 030 (0.40 g, 0.644 mmol) in anhydrous acetone (25.0 mL) was treated with NaI (0.39 g, 2.58 mmol) to provide 046 (0.35 g, 76%) as a dark blue color solid. ¹H NMR (500 MHz, CDCl₃) 7.47 (t, J=8.0 Hz, 1H), 7.23 (d, J=9.5 Hz, 2H), 7.20 (d, J=3.0 Hz, 2H), 6.71 (d, J=6.5 Hz, 1H), 6.69 (d, J=6.0 Hz, 1H), 6.64 (dd, J=9.5, 2.5 Hz, 2H), 3.68 (s, 6H), 3.40 (s, 12H), 3.14 (t, J=7.5 Hz, 2H), 1.79-1.70 (m, 2H), 1.30-1.22 (m, 2H), 0.73 (s, 3H) ppm; ¹³C NMR (125 MHz, CDCl₃) δ 167.14, 157.48, 157.44, 154.28, 146.79, 141.20, 131.15, 128.89, 120.75, 115.90, 114.24, 104.09, 103.91, 56.44, 56.27, 41.59, 28.06, 18.44, 11.23, −2.92 ppm; ¹⁹F NMR (470 MHz, CDCl₃); −75.04 ppm; HRMS (ESI) m/z: [M]⁺ calcd for C₂₉H₃₆IN₂O₂Si, 599.1585; found 599.1577.

### Example 64: (5s,10s)-3,7-Bis(dimethylamino)-5-(3-iodopropyl)-5-methyl-3′H,5H-spiro[dibenzo[b,e]siline-10,1′-isobenzofuran]-3′-one (047)

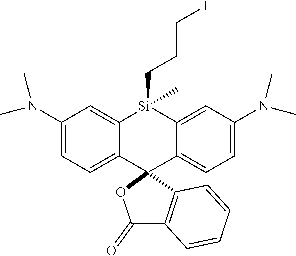

A solution of 037 (0.23 g, 0.47 mmol) in anhydrous acetone (15.0 mL) under argon atmosphere was treated with NaI (0.28 g, 1.87 mmol) at room temperature and reaction mixture was stirred at 80° C. for 18 h. After completion of the reaction, cooled to room temperature and solvent was evaporated under reduced pressure. The resulting residue was purified by flash column chromatography (Silicycle column, 25 g, 0-25% EtOAc in 20% v/v DCM/Hexanes, linear gradient for 20 min) to provide 047 (0.19 g, 73%) as a light green color solid. ¹H NMR (500 MHz, CDCl₃) δ 7.97 (dt, J=7.5, 1.0 Hz, 1H), 7.65 (td, J=7.5, 1.0 Hz, 1H), 7.56 (td, J=7.5, 1.0 Hz, 1H), 7.30 (d, J=7.5 Hz, 1H), 6.94 (br s, 2H), 6.73 (d, J=9.0 Hz, 2H), 6.55 (d, J=7.5 Hz, 2H), 3.17 (t, J=7.0 Hz, 2H), 2.97 (s, 12H), 1.95-1.86 (m, 2H), 1.24-1.17 (m, 2H), 0.65 (s, 3H) ppm; ¹³C NMR (125 MHz, CDCl₃) δ 170.61, 154.28, 149.39, 135.72, 133.81, 132.20, 128.92, 128.68, 127.52, 125.74, 124.82, 116.76, 113.57, 92.05, 40.43, 28.83, 18.40, 12.01, −3.45 ppm; HRMS (ESI) m/z: [M+H]⁺ calcd for C₂₈H₃₂IN₂O₂Si, 583.1272; found 583.1260.

### Example 65: (5r,10r)-3,7-Bis(dimethylamino)-5-(3-iodopropyl)-5-methyl-3′H,5H-spiro[dibenzo[b,e]siline-10,1′-isobenzofuran]-3′-one (048)

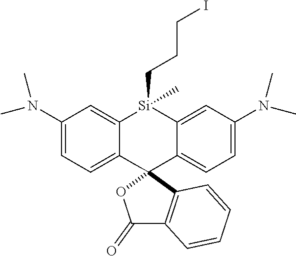

The same procedure was used as described above in Example 64. A solution of 038 (0.2 g, 0.41 mmol) in anhydrous acetone (12.0 mL) was treated with NaI (0.25 g, 1.64 mmol) to provide 048 (0.17 g, 71%) as a light green color solid. ¹H NMR (500 MHz, CDCl₃) δ 7.97 (dt, J=7.5, 0.5 Hz, 1H), 7.63 (td, J=7.5, 1.0 Hz, 1H), 7.53 (td, J=7.5, 0.5 Hz, 1H), 7.24 (d, J=7.5 Hz, 1H), 6.92 (br s, 2H), 6.77 (d, J=9.0 Hz, 2H), 6.57 (d, J=7.5 Hz, 2H), 3.25 (t, J=7.0 Hz, 2H), 2.97 (s, 12H), 1.97-1.88 (m, 2H), 1.30-1.24 (m, 2H), 0.60 (s, 3H) ppm; ¹³C NMR (125 MHz, CDCl₃) δ 170.88, 154.95, 149.36, 134.81, 134.03, 132.15, 128.88, 128.81, 127.03, 125.69, 124.41, 116.35, 113.85, 91.74, 40.45, 28.82, 16.84, 12.17, −1.16 ppm; HRMS (ESI) m/z: [M+H]⁺ calcd for C₂₈H₃₂IN₂O₂Si, 583.1272; found 583.1263.

Example 66: N-(10-(2,6-Dimethoxyphenyl)-7-(dimethylamino)-5-(3-((2-ethoxy-2-oxoethyl)thio)propyl)-5-methyldibenzo[b,e]silin-3(5H)-ylidene)-N-methylmethanaminium (041). A solution of 046 (0.11 g, 0.16 mmol) in anhydrous DMF (3.0 mL) under argon atmosphere was treated with DIPEA (81.0 μL, 0.46 mmol) and ethyl thioglycolate (25.0 μL, 0.23 mmol) at room temperature and reaction mixture was stirred at room temperature for 18 h. After completion of the reaction, solvent was evaporated under reduced pressure. The resulting residue was purified by flash column chromatography (Biotage column, 10 g, gradient elution with 0-10% MeOH/DCM) to provide 041 (90.0 mg, 94%) as a dark blue color solid. ¹H NMR (500 MHz, CDCl₃) 7.46 (t, J=8.5 Hz, 1H), 7.22 (d, J=9.5 Hz, 2H), 7.15 (d, J=2.5 Hz, 2H), 6.70 (t, J=8.0 Hz, 2H), 6.64 (dd, J=9.5, 2.5 Hz, 2H), 4.10 (q, J=7.5 Hz, 2H), 3.67 (s, 3H), 3.66 (s, 3H), 3.39 (s, 12H), 3.09 (s, 2H), 2.57 (t, J=7.5 Hz, 2H), 1.58-1.50 (m, 2H), 1.23 (t, J=7.5 Hz, 3H), 1.23-1.17 (m, 2H, overlapping), 0.70 (s, 3H) ppm; ¹³C NMR (125 MHz, CDCl₃) δ 170.63, 167.27, 157.48, 157.40, 154.25, 147.03, 141.22, 131.15, 128.91, 120.55, 115.88, 114.22, 104.08, 103.92, 61.43, 56.28, 41.50, 35.83, 33.96, 22.98, 16.09, 14.32, −3.12 ppm; HRMS (ESI) m/z: [M]⁺ calcd for C₃₃H₄₃N₂O₄SSi, 591.2707; found 591.2696.

### Example 67: Ethyl 2-((3-((5s,10s)-3,7-bis(dimethylamino)-5-methyl-3′-oxo-3′H,5H-spiro[dibenzo[b,e]siline-10,1′-isobenzofuran]-5-yl)propyl)thio)acetate (042)

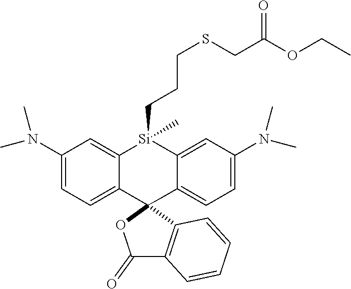

A solution of 047 (0.10 g, 0.17 mmol) in anhydrous DMF (3.0 mL) under argon atmosphere was treated with DIPEA (90.0 μL, 0.52 mmol) and ethyl thioglycolate (31.0 μL, 0.26 mmol) at room temperature and reaction mixture was stirred at room temperature for 18 h. After completion of the reaction, solvent was evaporated under reduced pressure. The resulting residue was purified by flash column chromatography (Biotage column, 10 g, gradient elution with 0-50% EtOAc/DCM, linear gradient for 20 minutes) to provide 042 (95.0 mg, 94%) as a light green color solid. ¹H NMR (500 MHz, CDCl₃) δ 7.97 (d, J=8.0 Hz, 1H), 7.65 (td, J=7.5, 1.0 Hz, 1H), 7.56 (td, J=7.0, 0.5 Hz, 1H), 7.29 (d, J=7.5 Hz, 1H), 6.95 (br s, 2H), 6.74 (d, J=9.0 Hz, 2H), 6.56 (br s, 2H), 4.13 (q, J=7.0 Hz, 2H), 3.15 (s, 2H), 2.97 (s, 12H), 2.62 (t, J=7.5 Hz, 2H), 1.75-1.66 (m, 2H), 1.24 (t, J=7.5 Hz, 3H), 1.26-1.18 (m, 2H, overlapping), 0.64 (s, 3H) ppm; ¹³C NMR (125 MHz, CDCl₃) δ 170.83, 170.62, 154.24, 149.30, 136.00, 133.82, 132.21, 128.92, 128.65, 127.46, 125.74, 124.82, 116.91, 113.54, 92.00, 61.38, 40.46, 36.19, 33.85, 23.90, 15.95, 14.30, −3.41 ppm; HRMS (ESI) m/z: [M+H]⁺ calcd for C₃₂H₃₉N₂O₄SSi, 575.2394; found 575.2382.

### Example 68: Ethyl 2-((3-((5r,10r)-3,7-bis(dimethylamino)-5-methyl-3′-oxo-3′H,5H-spiro[dibenzo[b,e]siline-10,1′-isobenzofuran]-5-yl)propyl)thio)acetate (043)

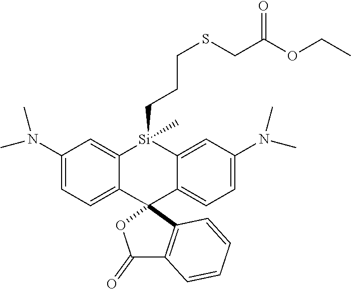

The same procedure was used as described above for compound 042. A solution of 048 (0.10 g, 0.17 mmol) in anhydrous DMF (3.0 mL) was treated with DIPEA (90.0 μL, 0.52 mmol) and ethyl thioglycolate (31.0 μL, 0.26 mmol) to provide 043 (92.0 mg, 93%) as a light green color solid. ¹H NMR (500 MHz, CDCl₃) δ 7.96 (d, J=8.0 Hz, 1H), 7.61 (td, J=7.5, 1.0 Hz, 1H), 7.52 (td, J=7.0, 0.5 Hz, 1H), 7.23 (d, J=8.0 Hz, 1H), 6.92 (br s, 2H), 6.78 (d, J=9.0 Hz, 2H), 6.57 (d, J=9.0 Hz, 2H), 4.13 (q, J=7.0 Hz, 2H), 3.15 (s, 2H), 2.96 (s, 12H), 2.70 (t, J=7.0 Hz, 2H), 1.80-1.71 (m, 2H), 1.29-1.23 (m, 2H), 1.24 (t, J=7.0 Hz, 3H, overlapping), 0.58 (s, 3H) ppm; ¹³C NMR (125 MHz, CDCl₃) δ 170.99, 170.63, 155.24, 149.33, 134.92, 134.05, 132.09, 128.81, 128.74, 126.84, 125.66, 124.32, 116.41, 113.85, 91.70, 61.41, 40.42, 36.38, 33.80, 23.99, 14.71, 14.30, −1.28 ppm; HRMS (ESI) m/z: [M+H]⁺ calcd for C₃₂H₃₉N₂O₄SSi, 575.2394; found 575.2382.

### Example 69: 2-((3-((5s,10s)-3,7-Bis(dimethylamino)-5-methyl-3′-oxo-3′H,5H-spiro[dibenzo[b,e]siline-10,1′-isobenzofuran]-5-yl)propyl)thio)acetic acid (063)

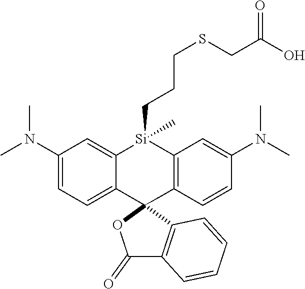

A solution of 042 (90.0 mg, 0.16 mmol) in a mixture of anhydrous MeOH/THF (1:1, 4.0 mL) under argon atmosphere was treated with 1M NaOH (0.32 mL, 0.32 mmol). The reaction mixture was then stirred at room temperature for 2 hours. Then the reaction mixture was acidified with 1M HCl (0.35 mL), diluted with H₂O (10.0 mL), and extracted with EtOAc (2×20.0 mL), the combined extracts were washed with saturated NaCl solution (25.0 mL), dried (Na₂SO₄), filtered, and evaporated under reduced pressure. The resulting residue was dried under high vacuum for four hours to provide crude acid 063 (85.0 mg, 97%) as a blue color gum. This acid was used for next step without further purification. HRMS (ESI) m/z: [M+H]⁺ calcd for C₃₀H₃₅N₂O₄SSi, 547.2081; found 547.2075.

### Example 70: 2,5-Dioxopyrrolidin-1-yl 2-((3-((5s,10s)-3,7-bis(dimethylamino)-5-methyl-3′-oxo-3′H,5H-spiro[dibenzo[b,e]siline-10,1′-isobenzofuran]-5-yl)propyl)thio)acetate (039)

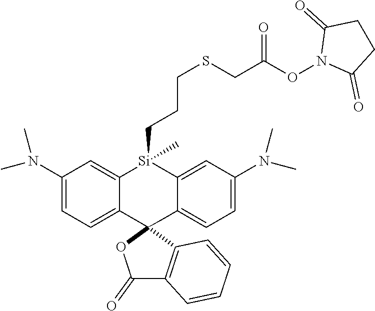

A solution of 063 (60.0 mg, 0.11 mmol) in anhydrous DMF (2.0 mL) under argon atmosphere was treated with TSTU (50.0 mg, 0.17 mmol) and DIPEA (58.0 μL, 0.33 mmol). After stirring the reaction at room temperature for 2 h, the reaction mixture was diluted with 10% w/v citric acid (5.0 mL), and extracted with EtOAc (2×10.0 mL), the combined extracts were washed with saturated NaCl solution (10.0 mL), dried (Na₂SO₄), filtered, and evaporated under reduced pressure. The resulting residue was purified by flash column chromatography (Silicycle column, 10 g, 0-100% EtOAc/DCM, linear gradient for 20 min) to provide 039 (50.0 mg, 70%) as a light blue color solid. ¹H NMR (500 MHz, CDCl₃) δ 7.96 (d, J=7.5 Hz, 1H), 7.64 (td, J=7.5, 1.0 Hz, 1H), 7.55 (td, J=7.0, 0.5 Hz, 1H), 7.28 (d, J=7.5 Hz, 1H), 6.98 (br s, 2H), 6.75 (d, J=8.5 Hz, 2H), 6.58 (br s, 2H), 3.38 (s, 2H), 2.97 (s, 12H), 2.84 (s, 4H), 2.71 (t, J=7.5 Hz, 2H), 1.78-1.69 (m, 2H), 1.26-1.19 (m, 2H), 0.64 (s, 3H) ppm; HRMS (ESI) m/z: [M+H]⁺ calcd for C₃₄H₃₈N₃O₆SSi, 644.2245; found 644.2248.

### Example 71: 2-((3-((5r,10r)-3,7-Bis(dimethylamino)-5-methyl-3′-oxo-3′H,5H-spiro[dibenzo[b,e]siline-10,1′-isobenzofuran]-5-yl)propyl)thio)acetic acid (064)

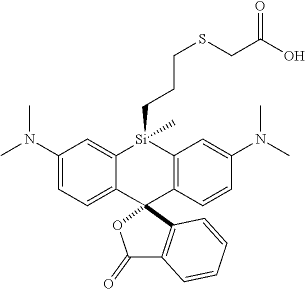

The same procedure was used as described above for compound 063. A solution of 043 (90.0 mg, 0.16 mmol) in a mixture of anhydrous MeOH/THF (1:1, 4.0 mL) was treated with 1M NaOH (0.32 mL, 0.32 mmol) to provide crude acid 064 (86.0 mg, 97%) as a blue color gum. HRMS (ESI) m/z: [M+H]⁺ calcd for C₃₀H₃₅N₂O₄SSi, 547.2081; found 547.2073.

### Example 72: 2,5-Dioxopyrrolidin-1-yl 2-((3-((5r,10r)-3,7-bis(dimethylamino)-5-methyl-3′-oxo-3′H,5H-spiro[dibenzo[b,e]siline-10,1′-isobenzofuran]-5-yl)propyl)thio)acetate (040)

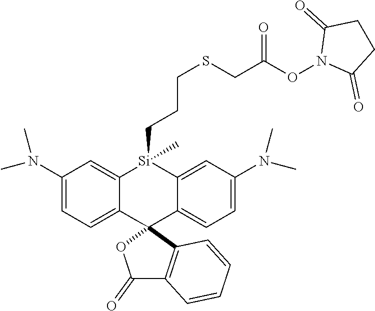

The same procedure was used as described above for compound 039. A solution of 064 (60.0 mg, 0.11 mmol) in anhydrous DMF (2.0 mL) was treated with TSTU (50.0 mg, 0.17 mmol) and DIPEA (58.0 μL, 0.33 mmol) to provide 040 (48.0 mg, 68%) as a light blue color solid. ¹H NMR (500 MHz, CDCl₃) δ 7.96 (d, J=7.5 Hz, 1H), 7.62 (td, J=7.0, 1.0 Hz, 1H), 7.53 (td, J=7.0, 0.5 Hz, 1H), 7.24 (d, J=8.0 Hz, 1H), 6.96 (br s, 2H), 6.79 (d, J=9.0 Hz, 2H), 6.60 (d, J=6.0 Hz, 2H), 3.41 (s, 2H), 2.97 (s, 12H), 2.83 (s, 4H), 2.79 (t, J=7.0 Hz, 2H), 1.82-1.74 (m, 2H), 1.32-1.26 (m, 2H), 0.59 (s, 3H) ppm; HRMS (ESI) m/z: [M+H]⁺ calcd for C₃₄H₃₈N₃O₆SSi, 644.2245; found 644.2261.

### Example 73: 2-((3-((5s,10s)-3,7-Bis(dimethylamino)-5-methyl-3′-oxo-3′H,5H-spiro[dibenzo[b,e]siline-10,1′-isobenzofuran]-5-yl)propyl)thio)-N-(2-(2-((6-chlorohexyl)oxy)ethoxy)ethyl)acetamide (044)

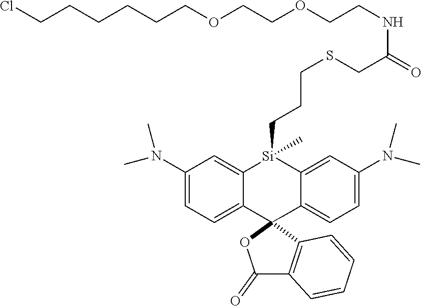

A solution of 067 (60.0 mg, 0.11 mmol) in anhydrous DMF (2.0 mL) under argon atmosphere was combined with HaloTag® amine (O2) ligand (55.0 mg, 0.16 mmol), and treated with HATU (71.0 mg, 0.19 mmol) and DIPEA (96.0 μL, 0.55 mmol). After stirring the reaction at room temperature for 4 h, the reaction mixture was diluted with 0.25M HCl (10.0 mL), and extracted with EtOAc (2×20.0 mL), the combined extracts were washed with saturated NaHCO₃ (15.0 mL), saturated NaCl solution (15.0 mL), dried (Na₂SO₄), filtered, and evaporated under reduced pressure. The resulting residue was purified by flash column chromatography (Silicycle column, 10 g, 25-100% EtOAc/DCM, linear gradient for 20 min) to provide 044 (40.0 mg, 48%) as a light green color solid. ¹H NMR (500 MHz, CD₃OD) δ 7.95 (d, J=7.5 Hz, 1H), 7.75 (td, J=7.5, 1.0 Hz, 1H), 7.64 (td, J=7.0, 0.5 Hz, 1H), 7.27 (d, J=7.5 Hz, 1H), 7.01 (d, J=3.0 Hz, 2H), 6.68 (d, J=8.5 Hz, 2H), 6.62 (dd, J=9.0, 3.0 Hz, 2H), 3.53-3.45 (m, 6H), 3.43 (t, J=5.5 Hz, 2H), 3.38 (t, J=6.5 Hz, 2H), 3.28 (t, J=5.5 Hz, 2H), 3.08 (s, 2H), 2.96 (s, 12H), 2.53 (t, J=7.5 Hz, 2H), 1.75-1.68 (m, 2H), 1.67-1.61 (m, 2H), 1.55-1.48 (m, 2H), 1.45-1.37 (m, 2H), 1.36-1.28 (m, 2H), 1.22-1.16 (m, 2H), 0.63 (s, 3H) ppm; ¹³C NMR (125 MHz, CD₃OD) δ 172.83, 172.61, 156.28, 151.03, 136.80, 135.50, 133.00, 130.27, 129.42, 127.95, 126.27, 125.87, 117.92, 114.83, 94.15, 72.17, 71.24, 71.15, 70.33, 45.72, 40.55, 36.99, 36.40, 33.74, 30.51, 27.73, 26.46, 25.29, 16.60, −3.07 ppm; HRMS (ESI) m/z: [M+h]⁺ calcd for C₄₀H₅₅ClN₃O₅SSi, 752.3315; found 752.3299.

### Example 74: 2-((3-((5r,10r)-3,7-Bis(dimethylamino)-5-methyl-3′-oxo-3′H,5H-spiro[dibenzo[b,e]siline-10,1′-isobenzofuran]-5-yl)propyl)thio)-N-(2-(2-((6-chlorohexyl)oxy)ethoxy)ethyl)acetamide (045)

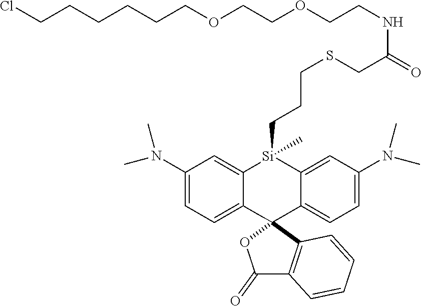

The same procedure was used as described above for compound 044. A solution of 064 (55.0 mg, 0.10 mmol) in anhydrous DMF (2.0 mL) was combined with HaloTag® amine (O2) ligand (51.0 mg, 0.15 mmol), treated with HATU (66.0 mg, 0.17 mmol) and DIPEA (88.0 μL, 0.51 mmol) to provide 045 (38.0 mg, 50%) as a light green color solid. ¹H NMR (500 MHz, CD₃OD) δ 7.95 (d, J=7.5 Hz, 1H), 7.76 (td, J=7.5, 1.0 Hz, 1H), 7.64 (td, J=7.0, 0.5 Hz, 1H), 7.28 (d, J=7.5 Hz, 1H), 7.02 (d, J=2.5 Hz, 2H), 6.69 (d, J=9.0 Hz, 2H), 6.64 (dd, J=9.0, 2.5 Hz, 2H), 3.53-3.46 (m, 6H), 3.43 (t, J=5.5 Hz, 2H), 3.39 (t, J=6.5 Hz, 2H), 3.27 (t, J=5.0 Hz, 2H), 3.10 (s, 2H), 2.97 (s, 12H), 2.63 (t, J=7.0 Hz, 2H), 1.75-1.63 (m, 4H), 1.56-1.49 (m, 2H), 1.45-1.37 (m, 2H), 1.36-1.28 (m, 4H), 0.55 (s, 3H) ppm; ¹³C NMR (125 MHz, CD₃OD) δ 172.96, 172.54, 156.38, 151.02, 136.29, 135.62, 132.94, 130.25, 129.66, 127.91, 126.29, 125.77, 117.69, 114.92, 94.19, 72.20, 71.22, 71.18, 70.35, 45.70, 40.52, 37.15, 36.38, 33.72, 30.52, 27.72, 26.47, 25.41, 14.94, −1.13 ppm; IRMS (ESI) m/z: [M+h]⁺ calcd for C₄₀H₅₅ClN₃O₅SSi, 752.3315; found 752.3300.

### Example 75: N-(5-(22-Chloro-7,10,13,16-tetraoxa-4-thiadocosyl)-10-(2,6-dimethoxyphenyl)-7-(dimethylamino)-5-methyldibenzo[b,e]silin-3(5H)-ylidene)-N-methylmethanaminium (049)

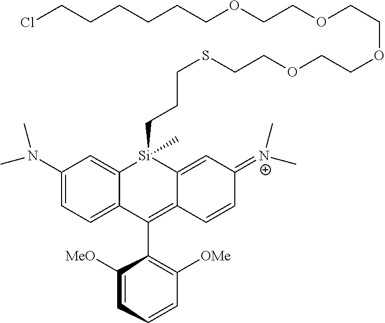

A solution of 046 (80.0 mg, 0.11 mmol) in anhydrous DMF (2.0 mL) under argon atmosphere was treated with DIPEA (59.0 μL, 0.34 mmol) and HaloTag® thiol (O4) ligand (55.0 μL, 0.17 mmol) at room temperature and reaction mixture was stirred at room temperature for 72 h. After completion of the reaction, solvent was evaporated under reduced pressure. The resulting residue was purified by flash column chromatography (Biotage column, 10 g, 0-15% MeOH/DCM, linear gradient for 20 min) to provide 049 (60.0 mg, 68%) as a dark blue color solid. ¹H NMR (500 MHz, CDCl₃) 7.46 (t, J=8.5 Hz, 1H), 7.23 (d, J=9.5 Hz, 2H), 7.13 (d, J=3.0 Hz, 2H), 6.71 (d, J=6.0 Hz, 1H), 6.69 (d, J=6.5 Hz, 1H), 6.63 (dd, J=9.5, 2.5 Hz, 2H), 3.67 (s, 3H), 3.66 (s, 3H), 3.66-3.61 (m, 8H), 3.60-3.54 (m, 6H), 3.52 (t, J=6.5 Hz, 2H), 3.46 (t, J=6.5 Hz, 2H), 3.35 (s, 12H), 2.60 (t, J=6.5 Hz, 2H), 2.46 (t, J=6.5 Hz, 2H), 1.80-1.72 (m, 2H), 1.62-1.55 (m, 2H), 1.54-1.47 (m, 2 H), 1.46-1.40 (m, 2H), 1.39-1.32 (m, 2H), 1.16-1.10 (m, 2H), 0.65 (s, 3H) ppm; ¹³C NMR (125 MHz, CDCl₃) δ 167.35, 157.50, 157.45, 154.26, 147.13, 141.25, 131.14, 128.93, 120.42, 115.91, 114.16, 104.09, 103.88, 71.39, 70.99, 70.68, 70.63, 70.61, 70.56, 70.28, 70.12, 56.26, 56.21, 45.24, 41.01, 35.51, 32.68, 31.29, 29.48, 26.84, 25.51, 23.66, 16.09, −3.53 ppm; HRMS (ESI) m/z: [m]⁺ calcd for C₄₃H₆₄ClN₂O₆SSi, 799.3937; found 799.3926.

### Example 76: (5s,10s)-5-(22-Chloro-7,10,13,16-tetraoxa-4-thiadocosyl)-3,7-bis(dimethylamino)-5-methyl-3′H,5H-spiro[dibenzo[b,e]siline-10,1′-isobenzofuran]-3′-one (050)

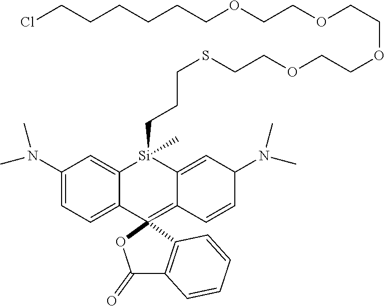

A solution of 047 (50.0 mg, 0.09 mmol) in anhydrous DMF (2.0 mL) under argon atmosphere was treated with DIPEA (45.0 μL, 0.26 mmol) and HaloTag® thiol (O4) ligand (42.0 μL, 0.13 mmol) at room temperature and reaction mixture was stirred at room temperature for 72 h. After completion of the reaction, solvent was evaporated under reduced pressure. The resulting residue was purified by flash column chromatography (Biotage column, 10 g, 0-80% EtOAc/DCM, linear gradient for 20 min) to provide 050 (50.0 mg, 75%) as a light green color solid. ¹H NMR (500 MHz, CD₃OD) 7.94 (d, J=7.5 Hz, 1H), 7.74 (td, J=7.5, 1.0 Hz, 1H), 7.63 (td, J=7.5, 1.0 Hz, 1H), 7.25 (d, J=7.5 Hz, 1H), 7.02 (d, J=2.5 Hz, 2H), 6.69 (d, J=9.0 Hz, 2H), 6.64 (dd, J=9.0, 3.0 Hz, 2H), 3.60-3.47 (m, 16H), 3.43 (t, J=6.5 Hz, 2H), 2.97 (s, 12H), 2.53 (t, J=6.5 Hz, 2H), 2.48 (t, J=7.0 Hz, 2H), 1.77-1.70 (m, 2H), 1.66-1.59 (m, 2H), 1.58-1.51 (m, 2H), 1.47-1.40 (m, 2H), 1.39-1.32 (m, 2H), 1.23-1.17 (m, 2H), 0.63 (s, 3H) ppm; ¹³C NMR (125 MHz, CD₃OD) δ 156.50, 151.05, 136.88, 135.50, 132.98, 130.21, 129.48, 127.84, 126.30, 125.80, 117.92, 114.94, 72.15, 71.57, 71.55, 71.54, 71.20, 71.18, 45.72, 40.51, 36.35, 33.76, 31.99, 30.56, 27.73, 26.50, 25.61, 16.26, −2.94 ppm; IRMS (ESI) m/z: [M+h]⁺ calcd for C₄₂H₆₀ClN₂O₆SSi, 783.3624; found 783.3612.

### Example 77: (5r,10r)-5-(22-Chloro-7,10,13,16-tetraoxa-4-thiadocosyl)-3,7-bis(dimethylamino)-5-methyl-3′H,5H-spiro[dibenzo[b,e]siline-10,1′-isobenzofuran]-3′-one (051)

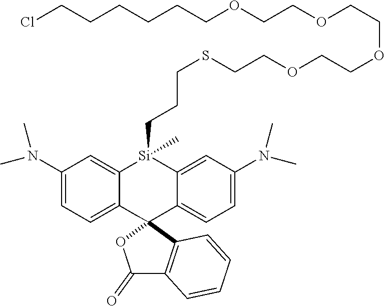

The same procedure was used as described above for compound 050. A solution of 048 (50.0 mg, 0.09 mmol) in anhydrous DMF (2.0 mL) under argon atmosphere was treated with DIPEA (45.0 μL, 0.26 mmol) and HaloTag® thiol (O4) ligand (42.0 μL, 0.13 mmol) to provide 051 (45.0 mg, 66%) as a light green color solid. ¹H NMR (500 MHz, CD₃OD) 7.95 (d, J=7.5 Hz, 1H), 7.75 (td, J=7.5, 1.0 Hz, 1H), 7.64 (td, J=7.5, 1.0 Hz, 1H), 7.29 (d, J=7.5 Hz, 1H), 7.02 (d, J=3.0 Hz, 2H), 6.69 (d, J=9.0 Hz, 2H), 6.64 (dd, J=9.0, 2.5 Hz, 2H), 3.58-3.47 (m, 16H), 3.42 (t, J=7.0 Hz, 2H), 2.97 (s, 12H), 2.60-2.53 (m, 4H), 1.77-1.62 (m, 4H), 1.58-1.50 (m, 2H), 1.47-1.28 (m, 6H), 0.55 (s, 3H) ppm; ¹³C NMR (125 MHz, CD₃OD) δ 172.99, 156.50, 151.00, 136.34, 135.63, 132.93, 130.24, 129.64, 127.88, 126.27, 125.74, 117.71, 114.97, 94.17, 72.19, 72.14, 71.59, 71.57, 71.56, 71.25, 71.19, 45.71, 40.52, 36.65, 33.75, 32.17, 30.56, 27.73, 26.50, 25.74, 14.72, −1.14 ppm; HRMS (ESI) m/z: [M+h]⁺ calcd for C₄₂H₆₀ClN₂O₆SSi, 783.3624; found 783.3617.

### Example 78: N-(5-(3-Azidopropyl)-10-(2,6-dimethoxyphenyl)-7-(dimethylamino)-5-methyldibenzo[b,e]silin-3(5H)-ylidene)-N-methylmethanaminium (052)

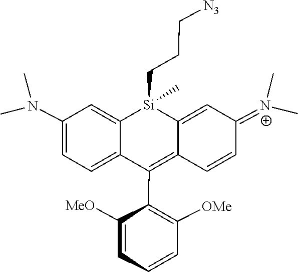

A solution of 046 (40.0 mg, 0.06 mmol) in anhydrous DMF (1.0 mL) under argon atmosphere was treated with NaN₃ (18.0 mg, 0.28 mmol) at room temperature and reaction mixture was stirred at room temperature for 12 h. After completion of the reaction, solvent was evaporated under reduced pressure. The resulting residue was purified by flash column chromatography (Biotage column, 10 g, gradient elution with 0-10% MeOH/DCM) to provide 052 (25.0 mg, 83%) as a dark blue color solid. ¹H NMR (500 MHz, CDCl₃) 7.46 (t, J=8.5 Hz, 1H), 7.22 (d, J=10.0 Hz, 2H), 7.17 (d, J=3.0 Hz, 2H), 6.70 (t, J=8.0 Hz, 2H), 6.63 (dd, J=9.5, 2.5 Hz, 2H), 3.67 (s, 3H), 3.64 (s, 3H), 3.38 (s, 12H), 3.18 (t, J=7.0 Hz, 2H), 1.56-1.47 (m, 2H), 1.19-1.13 (m, 2H), 0.72 (s, 3H) ppm; ¹³C NMR (125 MHz, CDCl₃) δ 167.23, 157.44, 157.35, 154.25, 146.82, 141.19, 131.15, 128.87, 120.54, 115.82, 114.21, 104.07, 103.90, 56.25, 56.19, 53.74, 41.51, 23.13, 14.07, −3.21 ppm; HRMS (ESI) m/z: [M]⁺ calcd for C₂₉H₃₆N₅O₂Si, 514.2633; found 514.2626.

### Example 79: (5s,10s)-5-(3-Azidopropyl)-3,7-bis(dimethylamino)-5-methyl-3′H,5H-spiro[dibenzo[b,e]siline-10,1′-isobenzofuran]-3′-one (053)

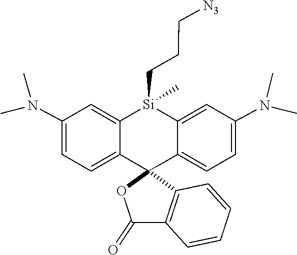

A solution of 047 (30.0 mg, 0.05 mmol) in anhydrous DMF (1.0 mL) under argon atmosphere was treated with NaN₃ (17.0 mg, 0.26 mmol) at room temperature and reaction mixture was stirred at room temperature for 12 h. After completion of the reaction, solvent was evaporated under reduced pressure. The resulting residue was purified by flash column chromatography (Biotage column, 10 g, gradient elution with 0-50% EtOAc/DCM, linear gradient for 20 minutes) to provide 053 (22.0 mg, 85%) as a light green color solid. ¹H NMR (500 MHz, CDCl₃) δ 7.97 (d, J=7.5 Hz, 1H), 7.67 (td, J=7.5, 1.0 Hz, 1H), 7.57 (td, J=7.0, 0.5 Hz, 1H), 7.31 (d, J=7.5 Hz, 1H), 6.95 (br s, 2H), 6.73 (d, J=9.0 Hz, 2H), 6.56 (d, J=7.0 Hz, 2H), 3.20 (t, J=7.0 Hz, 2H), 2.97 (s, 12H), 1.75-1.66 (m, 2H), 1.18-1.11 (m, 2H), 0.66 (s, 3H) ppm; ¹³C NMR (125 MHz, CDCl₃) δ 170.55, 154.12, 149.37, 135.93, 133.80, 132.21, 128.97, 128.73, 127.65, 125.74, 124.91, 116.82, 113.57, 92.09, 54.24, 40.24, 23.87, 13.80, −3.59 ppm; IRMS (ESI) m/z: [M+H]⁺ calcd for C₂₈H₃₂N₅O₂Si, 498.2320; found 498.2312.

### Example 80: (5r,10r)-5-(3-Azidopropyl)-3,7-bis(dimethylamino)-5-methyl-3′H,5H-spiro[dibenzo[b,e]siline-10,1′-isobenzofuran]-3′-one (054)

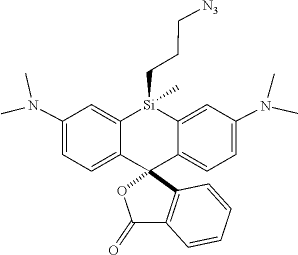

The same procedure was used as described above for compound 053. A solution of 048 (50.0 mg, 0.09 mmol) in anhydrous DMF (1.0 mL) was treated with NaN₃ (28.0 mg, 0.43 mmol) to provide 054 (38.0 mg, 88%) as a light green color solid. ¹H NMR (500 MHz, CDCl₃) δ 7.97 (d, J=7.5 Hz, 1H), 7.62 (td, J=7.0, 0.5 Hz, 1H), 7.53 (td, J=7.0, 0.5 Hz, 1H), 7.22 (d, J=7.5 Hz, 1H), 6.92 (br s, 2H), 6.78 (d, J=8.5 Hz, 2H), 6.58 (d, J=7.5 Hz, 2H), 3.28 (t, J=6.5 Hz, 2H), 2.97 (s, 12H), 1.79-1.69 (m, 2H), 1.24-1.16 (m, 2H), 0.60 (s, 3H) ppm; ¹³C NMR (125 MHz, CDCl₃) δ 170.89, 155.10, 149.34, 134.77, 134.07, 132.07, 128.93, 128.83, 126.95, 125.71, 124.31, 116.27, 113.92, 91.66, 54.40, 40.42, 24.14, 12.54, −1.29 ppm; HRMS (ESI) m/z: [M+H]⁺ calcd for C₂₈H₃₂N₅O₂Si, 498.2320; found 498.2312.

### Example 81: N-(10-(2,6-Dimethoxyphenyl)-7-(dimethylamino)-5-(3-(8-(4-((2,5-dioxopyrrolidin-1-yl)oxy)-4-oxobutanoyl)-8,9-dihydro-3H-dibenzo[b,f][1,2,3]triazolo[4,5-d]azocin-3-yl)propyl)-5-methyldibenzo[b,e]silin-3(5H)-ylidene)-N-methylmethanaminium (055)

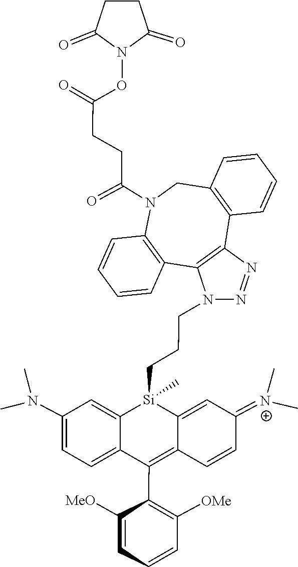

A solution of 052 (10.0 mg, 0.016 mmol) in anhydrous DMF (0.5 mL) under argon atmosphere was treated with DBCO-NHS-Ester (8.0 mg, 0.019 mmol) at room temperature and reaction mixture was stirred at room temperature for 2 h. After completion of the reaction, solvent was evaporated under reduced pressure. The resulting residue was purified by flash column chromatography (Biotage column, 10 g, 0-15% MeOH/DCM, linear gradient for 20 min) to provide 055 as a mixture of regioisomers (13.5 mg, 94%) in dark blue color solid. Although the NMR spectra were not interpretable, HRMS analyses were consistent with the expected product mixture. HRMS (ESI) m/z: [M]⁺ calcd for C₅₂H₅₄N₇₀₇Si, 916.3849; found 916.3843.

### Example 82: 2,5-Dioxopyrrolidin-1-yl 4-(3-(3-((5s,10s)-3,7-bis(dimethylamino)-5-methyl-3′-oxo-3′H,5H-spiro[dibenzo[b,e]siline-10,1′-isobenzofuran]-5-yl)propyl)-3,9-dihydro-8H-dibenzo[b,f][1,2,3]triazolo[4,5-d]azocin-8-yl)-4-oxobutanoate (056)

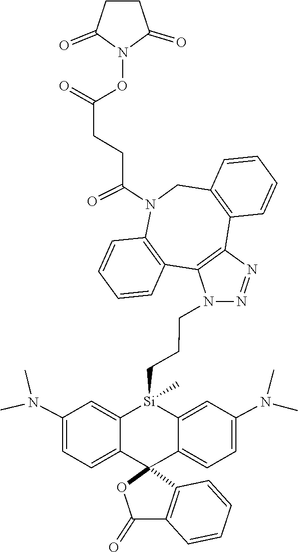

A solution of 053 (10.0 mg, 0.02 mmol) in anhydrous DMF (0.5 mL) under argon atmosphere was treated with DBCO-NHS-Ester (10.0 mg, 0.024 mmol) at room temperature and reaction mixture was stirred at room temperature for 2 h. After completion of the reaction, solvent was evaporated under reduced pressure. The resulting residue was purified by flash column chromatography (Biotage column, 10 g, 0-80% EtOAc/DCM, linear gradient for 20 min) to provide 056 as a mixture of regioisomers (17.0 mg, 97%) in light green color solid. Although the NMR spectra were not interpretable, HRMS analyses were consistent with the expected product mixture. HRMS (ESI) m/z: [M+H]⁺ calcd for C₅₁H₅₀N₇O₇Si, 900.3536; found 900.3528.

### Example 83: 2,5-Dioxopyrrolidin-1-yl 4-(3-(3-((5r,10r)-3,7-bis(dimethylamino)-5-methyl-3′-oxo-3′H,5H-spiro[dibenzo[b,e]siline-10,1′-isobenzofuran]-5-yl)propyl)-3,9-dihydro-8H-dibenzo[b,f][1,2,3]triazolo[4,5-d]azocin-8-yl)-4-oxobutanoate (057)

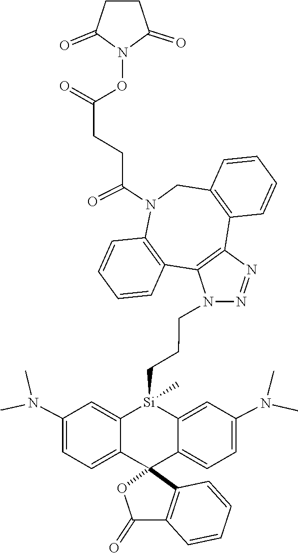

The same procedure was used as described above for compound 056. A solution of 054 (25.0 mg, 0.05 mmol) in anhydrous DMF (1.0 mL) was treated with DBCO-NHS-Ester (24.0 mg, 0.06 mmol) to provide 057 as a mixture of regioisomers (42.0 mg, 96%) in light green color solid. Although the NMR spectra were not interpretable, HRMS analyses were consistent with the expected product mixture. HRMS (ESI) m/z: [M+H]⁺ calcd for C₅₁H₅₀N₇O₇Si, 900.3536; found 900.3528.

### Example 84: N-(5-(3-(8-(3-((tert-butoxycarbonyl)amino)propanoyl)-8,9-dihydro-3H-dibenzo[b,f][1,2,3]triazolo[4,5-d]azocin-3-yl)propyl)-10-(2,6-dimethoxyphenyl)-7-(dimethylamino)-5-methyldibenzo[b,e]silin-3(5H)-ylidene)-N-methylmethanaminium (058)

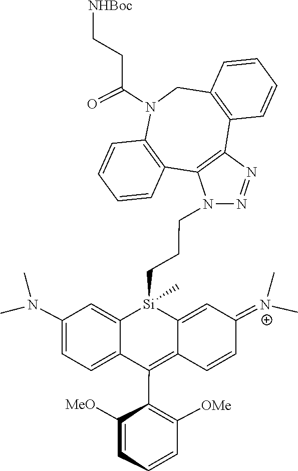

A solution of 052 (30.0 mg, 0.048 mmol) in anhydrous DMF (1.0 mL) under argon atmosphere was treated with DBCO-NH-Boc (22.0 mg, 0.057 mmol) at room temperature and reaction mixture was stirred at room temperature for 2 h. After completion of the reaction, solvent was evaporated under reduced pressure. The resulting residue was purified by flash column chromatography (Biotage column, 10 g, gradient elution with 0-10% MeOH/DCM, linear gradient for 20 min) to provide 058 as a mixture of regioisomers (39.0 mg, 91%) in dark blue color solid. Although the NMR spectra were not interpretable, HRMS analyses were consistent with the expected product mixture. HRMS (ESI) m/z: [M]⁺ calcd for C₅₂H₆₀N₇O₅Si, 890.4420; found 890.4427.

### Example 85: tert-Butyl (3-(3-(3-((5s,10s)-3,7-bis(dimethylamino)-5-methyl-3′-oxo-3′H,5H-spiro[dibenzo[b,e]siline-10,1′-isobenzofuran]-5-yl)propyl)-3,9-dihydro-8H-dibenzo[b,f][1,2,3]triazolo[4,5-d]azocin-8-yl)-3-oxopropyl)carbamate (059)

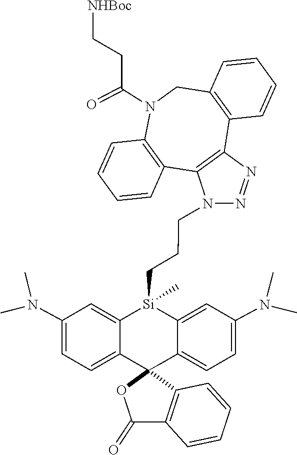

A solution of 053 (20.0 mg, 0.04 mmol) in anhydrous DMF (1.0 mL) under argon atmosphere was treated with DBCO-NH-Boc (18.0 mg, 0.048 mmol) at room temperature and reaction mixture was stirred at room temperature for 2 h. After completion of the reaction, solvent was evaporated under reduced pressure. The resulting residue was purified by flash column chromatography (Biotage column, 10 g, 0-80% EtOAc/DCM, linear gradient for 20 min) to provide 059 as a mixture of regioisomers (33.0 mg, 95%) in light green color solid. Although the NMR spectra were not interpretable, HRMS analyses were consistent with the expected product mixture. HRMS (ESI) m/z: [M+H]⁺ calcd for C₅₁H₅₆N₇O₅Si, 874.4107; found 874.4119.

### Example 86: tert-Butyl (3-(3-(3-((5r,10r)-3,7-bis(dimethylamino)-5-methyl-3′-oxo-3′H,5H-spiro[dibenzo[b,e]siline-10,1′-isobenzofuran]-5-yl)propyl)-3,9-dihydro-8H-dibenzo[b,f][1,2,3]triazolo[4,5-d]azocin-8-yl)-3-oxopropyl)carbamate (060)

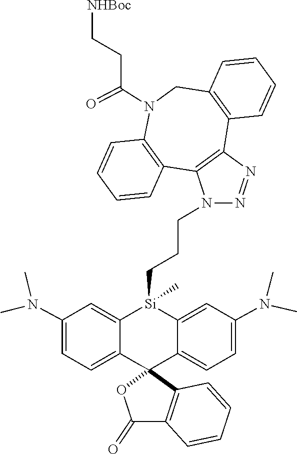

The same procedure was used as described above for compound 059. A solution of 054 (20.0 mg, 0.04 mmol) in anhydrous DMF (1.0 mL) was treated with DBCO-NH-Boc (18.0 mg, 0.048 mmol) to provide 060 as a mixture of regioisomers (32.0 mg, 92%) in light green color solid. Although the NMR spectra were not interpretable, HRMS analyses were consistent with the expected product mixture. HRMS (ESI) m/z: [M+H]⁺ calcd for C₅₁H₅₆N₇O₅Si, 874.4107; found 874.4120.

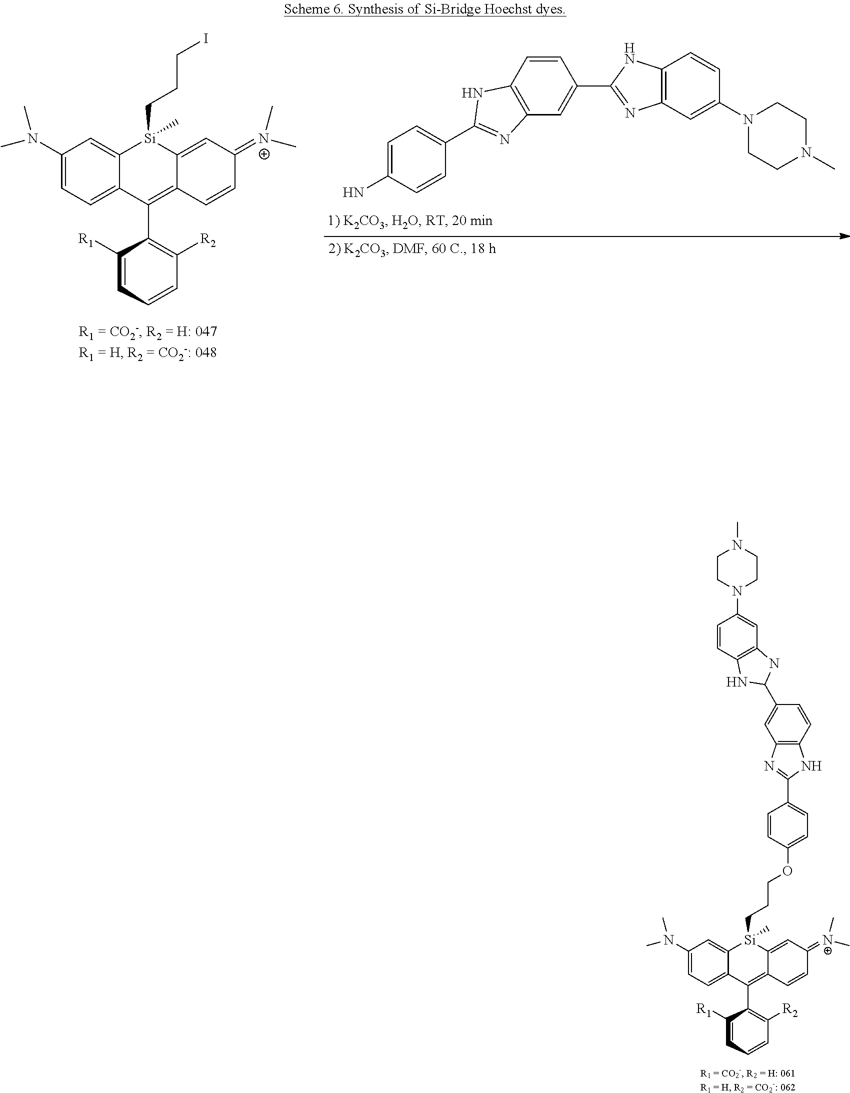

Scheme 6. Synthesis of Si-Bridge Hoechst dyes.

### Example 87: (5s,10s)-3,7-Bis(dimethylamino)-5-methyl-5-(3-(4-(5-(4-methylpiperazin-1-yl)-1H,1′H-[2,5′-bibenzo[d]imidazol]-2′-yl)phenoxy)propyl)-3′H,5H-spiro[dibenzo[b,e]siline-10,1′-isobenzofuran]-3′-one (061)

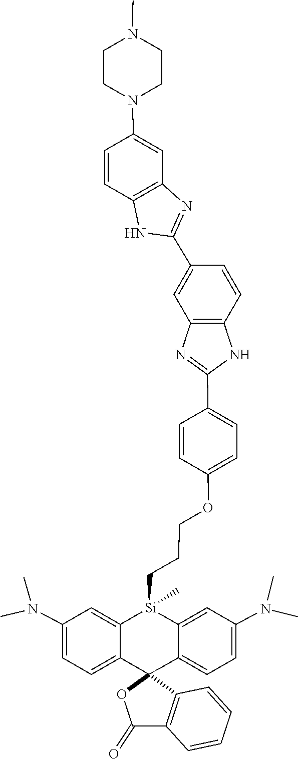

A solution of Hoechst 33258 trihydrochloride (0.15 g, 0.28 mmol) in H₂O (9.0 mL) under argon atmosphere was treated with a solution of K₂CO₃ (0.12 g, 0.84 mmol) in H₂O (3.0 mL). The reaction mixture was then stirred at room temperature for 20 min. The precipitate thus formed was isolated by filtration, washed with H₂O (5.0 mL) and high vacuumed for 24 hours to provide free base of Hoechst 33258 (110.0 mg, 93%) as an off-white color solid. The Hoechst 33258 free base (40.0 mg, 0.094 mmol) in anhydrous DMF (2.0 mL) was treated with K₂CO₃ (39.0 mg, 0.28 mmol) and 047 (66.0 mg, 0.11 mmol). After stirring the reaction at 60° C. for 18 h, solvent was evaporated under reduced pressure, the residue was dissolved in a mixture of MeOH/DCM (1:1 10.0 mL), and filtered through a small pad of celite to remove excess K₂CO₃. The resulting residue was purified by flash column chromatography (Biotage column, 10 g, gradient elution with 0-15% MeOH/DCM with constant 0.1% v/v TEA additive) to provide 061 (50.0 mg, 61%) as a light green color solid. ¹H NMR (500 MHz, CD₃OD) δ 8.23 (s, 1H), 7.96 (d, J=8.0 Hz, 2H), 7.94 (d, J=8.5 Hz, 2H), 7.74 (td, J=7.5, 1.0 Hz, 1H), 7.70-7.63 (m, 1H), 7.64 (td, J=7.0, 0.5 Hz, 1H, overlapping), 7.50 (d, J=9.0 Hz, 1H), 7.25 (d, J=7.5 Hz, 1H), 7.13 (s, 1H), 7.03 (dd, J=9.0, 2.0 Hz, 1H), 7.00 (d, J=2.5 Hz, 2H), 6.87 (d, J=8.5 Hz, 2H), 6.69 (d, J=9.0 Hz, 2H), 6.60 (dd, J=9.0, 3.0 Hz, 2H), 3.85 (t, J=6.5 Hz, 2H), 3.22 (t, J=4.5 Hz, 4H), 2.90 (s, 12H), 2.67 (t, J=4.5 Hz, 4H), 2.37 (s, 3H), 1.90-1.81 (m, 2H), 1.32-1.25 (m, 2H), 0.65 (s, 3H) ppm; ¹³C NMR (125 MHz, CD₃OD) δ 173.05, 162.58, 156.53, 155.43, 151.00, 149.58, 141.60, 136.71, 135.57, 132.83, 130.24, 129.54, 129.41, 127.90, 126.25, 125.78, 125.68, 122.54, 117.78, 116.05, 114.90, 94.44, 70.79, 56.16, 51.80, 46.07, 40.39, 24.89, 13.01, −2.95 ppm; HRMS (ESI) m/z: [M+H]⁺ calcd for C₅₃H₅₅N₈O₃Si, 879.4161; found 879.4155.

### Example 88: (5r,10r)-3,7-Bis(dimethylamino)-5-methyl-5-(3-(4-(5-(4-methylpiperazin-1-yl)-1H,1′H-[2,5′-bibenzo[d]imidazol]-2′-yl)phenoxy)propyl)-3′H,5H-spiro[dibenzo[b,e]siline-10,1′-isobenzofuran]-3′-one (062)

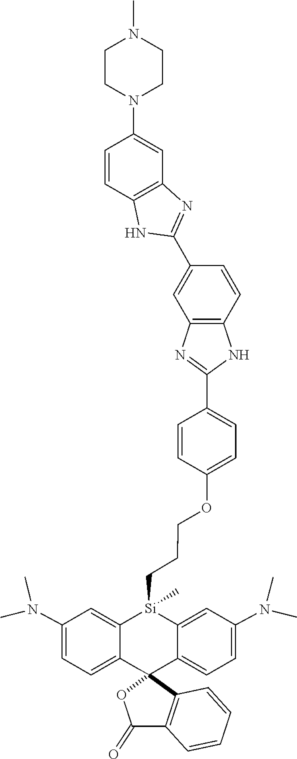

The same procedure was used as described above for compound 061. A solution of Hoechst 33258 free base (40.0 mg, 0.094 mmol) in anhydrous DMF (2.0 mL) was treated with K₂CO₃ (39.0 mg, 0.28 mmol) and 048 (66.0 mg, 0.11 mmol) to provide 062 (47.0 mg, 56%) as a light green color solid. ¹H NMR (500 MHz, CD₃OD) δ 8.25 (s, 1H), 8.00 (d, J=9.0 Hz, 2H), 7.94 (d, J=7.5 Hz, 2H), 7.71 (td, J=7.5, 1.0 Hz, 1H), 7.70-7.66 (m, 1H, overlapping), 7.61 (td, J=7.5, 1.0 Hz, 1H), 7.52 (d, J=8.5 Hz, 1H), 7.29 (d, J=8.0 Hz, 1H), 7.16 (s, 1H), 7.04 (dd, J=8.5, 2.0 Hz, 1H), 7.01 (d, J=3.0 Hz, 2H), 6.94 (d, J=9.0 Hz, 2H), 6.69 (d, J=9.0 Hz, 2H), 6.60 (dd, J=9.0, 3.0 Hz, 2H), 3.98 (t, J=6.5 Hz, 2H), 3.29 (t, J=4.5 Hz, 4H), 2.94 (s, 12H), 2.88 (t, J=4.5 Hz, 4H), 2.53 (s, 3H), 1.97-1.88 (m, 2H), 1.41-1.34 (m, 2H), 0.59 (s, 3H) ppm; ¹³C NMR (125 MHz, CD₃OD) δ 172.66, 161.96, 155.58, 154.93, 153.63, 150.44, 148.35, 136.11, 135.17, 132.36, 129.87, 129.51, 129.19, 127.70, 126.04, 125.45, 124.87, 122.47, 122.20, 117.29, 116.20, 115.73, 114.52, 94.24, 70.83, 55.34, 50.72, 45.05, 40.49, 24.80, 11.44, −1.02 ppm; HRMS (ESI) m/z: [M+H]⁺ calcd for C₅₃H₅₅N₈O₃Si, 879.4161; found 879.4155.

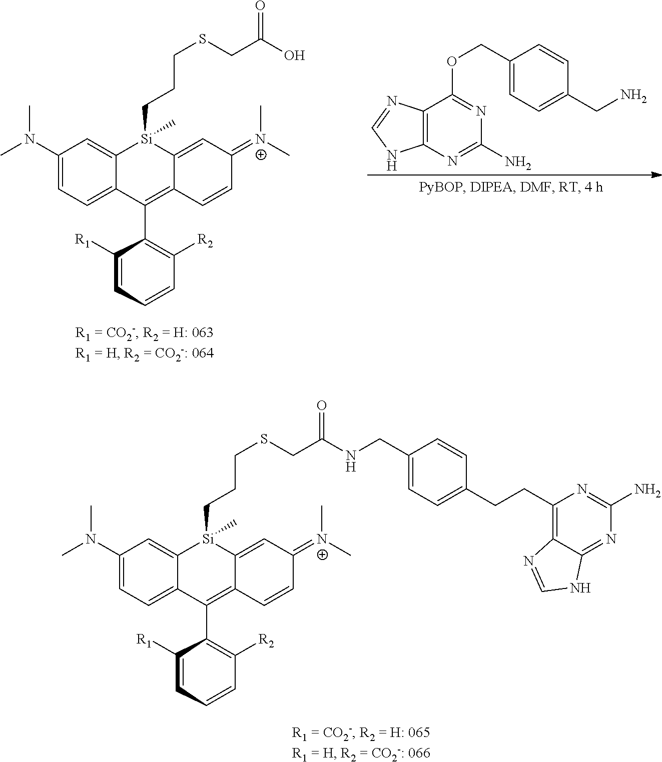

Scheme 7. Synthesis of Si-Bridged SNAP-tag Ligand.

### Example 89: N-(4-(((2-Amino-9H-purin-6-yl)oxy)methyl)benzyl)-2-((3-((5s,10s)-3,7-bis(dimethylamino)-5-methyl-3′-oxo-3′H,5H-spiro[dibenzo[b,e]siline-10,1′-isobenzofuran]-5-yl)propyl)thio)acetamide (065)

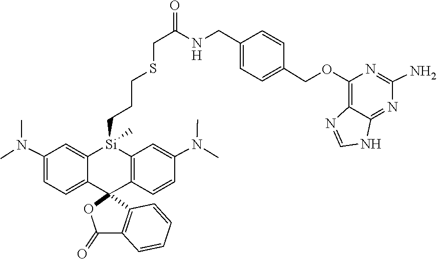 [SMILES: CN(C)c1ccc2c(c1)[Si@](C)(CCCSCC(=O)NCc1ccc(COc3nc(N)nc4[nH]cnc34)cc1)c1cc(N(C)C)ccc1[C@]21OC(=O)c2ccccc21]

A solution of 063 (50.0 mg, 0.092 mmol) in anhydrous DMF (1.5 mL) under argon atmosphere was combined with SNAP-tag ligand (37.0 mg, 0.14 mmol), and treated with PyBOP (71.0 mg, 0.14 mmol) and DIPEA (80.0 μL, 0.46 mmol). After stirring the reaction at room temperature for 4 h, solvent was evaporated under reduced pressure. The resulting residue was purified by flash column chromatography (Silicycle column, 10 g, 0-15% MeOH in 0.1% v/v TEA/DCM, linear gradient for 20 min)) to provide 065 (49.0 mg, 67%) as a light green color solid. ¹H NMR (500 MHz, CDCl₃) δ 8.08 (t, J=5.0 Hz, 1H), 7.95 (d, J=7.5 Hz, 1H), 7.68 (t, J=7.5 Hz, 1H), 7.57 (t, J=7.5 Hz, 1H), 7.33 (d, J=7.5 Hz, 1H), 7.28 (d, J=8.0 Hz, 2H), 7.21 (d, J=8.0 Hz, 2H), 6.93 (d, J=3.0 Hz, 2H), 6.68 (d, J=8.5 Hz, 2H), 6.50 (dd, J=9.0, 3.0 Hz, 2H), 5.31 (s, 2H), 4.38 (d, J=5.5 Hz, 2H), 3.31 (s, 2H), 2.95 (s, 12H), 2.50 (t, J=7.0 Hz, 2H), 1.75-1.66 (m, 2H), 1.19-1.13 (m, 2H), 0.62 (s, 3H) ppm; ¹³C NMR (125 MHz, CDCl₃) δ 170.97, 170.35, 159.21, 154.09, 149.45, 139.12, 136.41, 134.92, 133.95, 131.84, 129.68, 129.07, 128.71, 128.10, 127.70, 125.68, 125.10, 116.85, 113.32, 92.91, 68.70, 43.87, 40.34, 36.11, 36.01, 24.22, 16.00, −3.39 ppm; HRMS (ESI) m/z: [M+H]⁺ calcd for C₄₃H₄₇N₈O₄SSi, 799.3205; found 799.3216.

### Example 90: N-(4-(((2-Amino-9H-purin-6-yl)oxy)methyl)benzyl)-2-((3-((5r,10r)-3,7-bis(dimethylamino)-5-methyl-3′-oxo-3′H,5H-spiro[dibenzo[b,e]siline-10,1′-isobenzofuran]-5-yl)propyl)thio)acetamide (066)

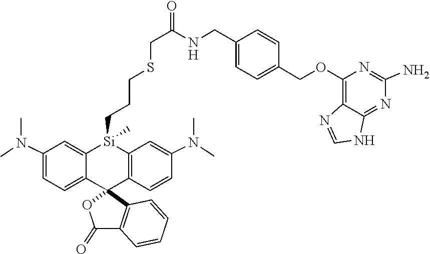 [SMILES: CN(C)c1ccc2c(c1)[Si@](C)(CCCSCC(=O)NCc1ccc(COc3nc(N)nc4[nH]cnc34)cc1)c1cc(N(C)C)ccc1[C@@]21OC(=O)c2ccccc21]

The same procedure was used as described above for compound 065. A solution of 064 (50.0 mg, 0.092 mmol) in anhydrous DMF (1.5 mL) was combined with SNAP-tag ligand (37.0 mg, 0.14 mmol), treated with PyBOP (71.0 mg, 0.14 mmol) and DIPEA (80.0 L, 0.46 mmol) to provide 066 (51.0 mg, 69%) as a light green color solid. ¹H NMR (500 MHz, CDCl₃) δ 7.94 (d, J=7.5 Hz, 1H), 7.62 (t, J=5.0 Hz, 1H), 7.59 (t, J=7.5 Hz, 1H), 7.50 (t, J=7.0 Hz, 1H), 7.35 (d, J=8.0 Hz, 2H), 7.24 (d, J=8.0 Hz, 2H), 7.20 (d, J=8.0 Hz, 1H), 6.90 (d, J=3.0 Hz, 2H), 6.76 (d, J=9.0 Hz, 2H), 6.54 (dd, J=8.5, 2.5 Hz, 2H), 5.36 (s, 2H), 4.41 (d, J=5.5 Hz, 2H), 3.29 (s, 2H), 2.93 (s, 12H), 2.65 (t, J=7.0 Hz, 2H), 1.79-1.71 (m, 2H), 1.26-1.19 (m, 2H), 0.54 (s, 3H) ppm; ¹³C NMR (125 MHz, CDCl₃) δ 171.06, 169.97, 159.20, 154.97, 149.37, 138.76, 135.10, 135.04, 134.11, 132.00, 129.63, 128.81, 127.89, 126.88, 125.68, 124.40, 116.44, 113.79, 92.19, 68.62, 43.77, 40.39, 36.84, 36.23, 24.26, 14.61, −1.30 ppm; IRMS (ESI) m/z: [M+H]⁺ calcd for C₄₃H₄₇N₈O₄SSi, 799.3205; found 799.3209.

### Example 91: Tri-Iodation of Compound 029

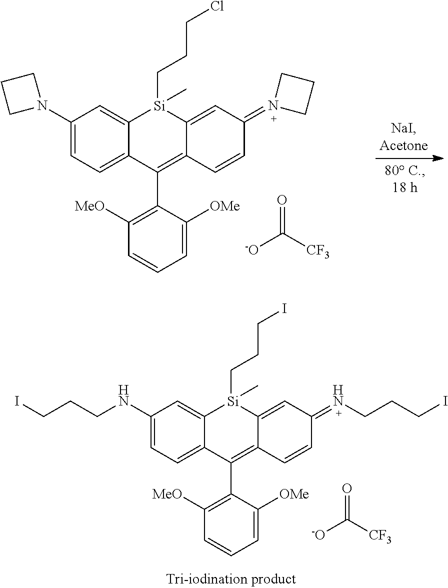

A solution of compound 029 (0.10 g, 0.16 mmol) in anhydrous acetone (5.0 mL) under argon atmosphere was treated with NaI (93.0 mg, 0.62 mmol) at room temperature and reaction mixture was stirred at 80° C. for 18 h. After completion of the reaction, the mixture was cooled to room temperature and the solvent was evaporated under reduced pressure. The resulting residue was purified by flash column chromatography (Silicycle column, 25 g, 0-10% MeOH in 1% v/v TFA/DCM, linear gradient for 20 min) provided unexpected tri-iodinated product of compound 029 (0.13 g, 92%) in dark blue color solid. ¹H NMR (500 MHz, CDCl₃) δ 7.46 (t, J=8.0 Hz, 1H), 7.21 (d, J=9.0 Hz, 2H), 7.16 (br s, 2H), 6.70 (d, J=7.5 Hz, 1H), 6.69 (d, J=7.0 Hz, 1H), 6.60 (d, J=9.5 Hz, 2H), 3.69 (s, 6H), 3.55 (t, J=6.5 Hz, 4H), 3.28 (t, J=6.5 Hz, 4H), 3.08 (t, J=7.0 Hz, 2H), 2.20 (p, J=6.5 Hz, 4H), 1.77-1.69 (m, 2H), 1.13-1.05 (m, 2H), 0.61 (s, 3H) ppm; ¹⁹F NMR (470 MHz, CDCl₃) δ −75.66 ppm; HRMS (ESI) m/z: [M]⁺ calcd for C₃₁H₃₉I₃N₂O₂Si, 878.9831; found 878.9822.

### Example 92: Ethyl 2-((3-((5r,10r)-3,7-di(azetidin-1-yl)-5-methyl-3′-oxo-3′H,5H-spiro[dibenzo[b,e]siline-10,1′-isobenzofuran]-5-yl)propyl)thio)acetate

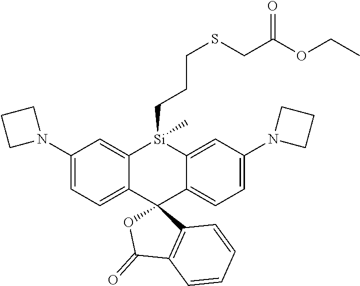 [SMILES: CCOC(=O)CSCCC[Si@]1(C)c2cc(N3CCC3)ccc2[C@@]2(OC(=O)c3ccccc32)c2ccc(N3CCC3)cc21]

The same procedure was used as described above for compound 042. A solution of 070 (45.0 mg, 0.074 mmol) in anhydrous DMF (1.0 mL) was treated with DIPEA (40.0 μL, 0.23 mmol) and ethyl thioglycolate (17.0 μL, 0.15 mmol) to provide the product (40.0 mg, 91%) as a light green color solid. ¹H NMR (500 MHz, CDCl₃) δ 7.96 (d, J=7.5 Hz, 1H), 7.63 (td, J=8.0, 1.0 Hz, 1H), 7.53 (td, J=7.5, 0.5 Hz, 1H), 7.23 (d, J=7.5 Hz, 1H), 6.78 (d, J=9.0 Hz, 2H), 6.70 (br s, 2H), 6.35 (br s, 2H), 4.14 (q, J=7.0 Hz, 2H), 3.94 (t, J=7.0 Hz, 8H), 3.16 (s, 2H), 2.69 (t, J=7.0 Hz, 2H), 2.39 (p, J=7.0 Hz, 4H), 1.75-1.67 (m, 2H), 1.28-1.21 (m, 2H), 1.25 (t, J=7.0 Hz, 3H, overlapping), 0.57 (s, 3H) ppm; HRMS (ESI) m/z: [M+H]⁺ calcd for C₃₄H₃₉N₂O₄SSi, 599.2394; found 599.2399.

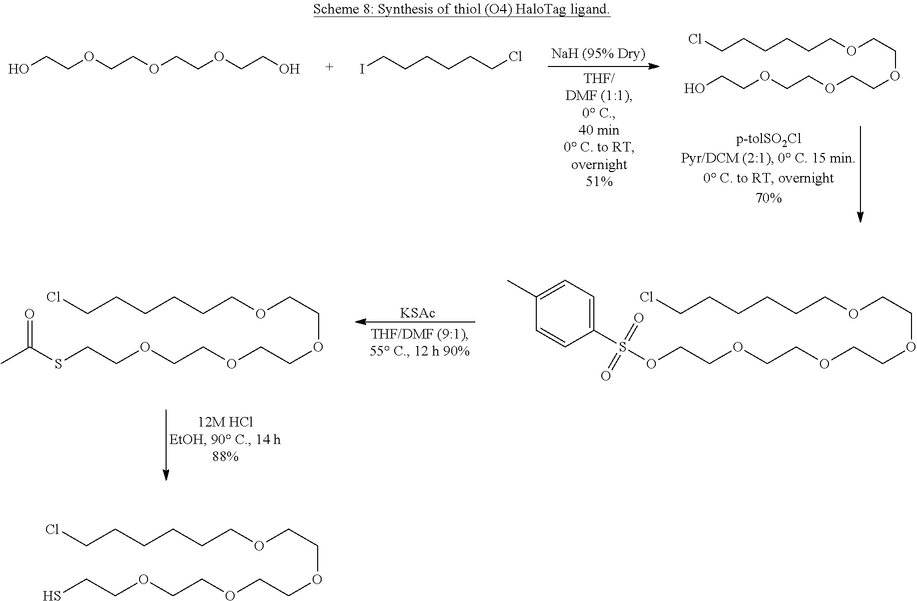

Scheme 8: Synthesis of thiol (O4) HaloTag ligand.

### Example 93: 18-Chloro-3,6,9,12-tetraoxaoctadecan-1-ol

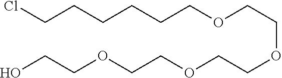 [SMILES: OCCOCCOCCOCCOCCCCCCCl]

A solution of NaH (dry 950) (0.49 g, 20.3 mmol) in a mixture of anhydrous THF/DMF (1:1, 80.0 mL) under argon atmosphere was cooled to 0° C. in an ice-water bath. After 10 min, tetraethylene glycol (7.04 mL, 40.6 mmol) was added dropwise. The resulting reaction mixture was stirred at 0° C. for 40 min. At the same temperature, 1-chloro-6-iodohexane (1.23 mL, 8.12 mmol) was added dropwise. The reaction mixture was then warmed to room temperature and stirred overnight. The reaction mixture was quenched with H₂O (10.0 mL), diluted with 1M HCl (50.0 mL) and extracted with CHCl₃ (2×100 mL), the combined extracts were washed with saturated NaCl solution (50.0 mL), dried (Na₂SO₄), filtered, and evaporated under reduced pressure. The resulting residue was purified by flash column chromatography (Silicycle column, 50 g, 25-100% EtOAc/Hexanes, linear gradient for 20 min) to provide 18-chloro-3,6,9,12-tetraoxaoctadecan-1-ol (1.30 g, 51%) as a color less oil. ¹H NMR (500 MHz, CDCl3) δ 3.74-3.70 (m, 2H), 3.69-3.63 (m, 10H), 3.62-3.60 (m, 2H), 3.59-3.56 (m, 2H), 3.53 (t, J=6.5 Hz, 2H), 3.45 (t, J=6.5 Hz, 2H), 2.45 (br s, 1H), 1.81-1.73 (m, 2H), 1.63-1.55 (m, 2H), 1.48-1.42 (m, 2H), 1.40-1.33 (m, 2H) ppm; ¹³C NMR (125 MHz, CDCl₃) δ 72.62, 71.38, 70.77, 70.75, 70.73, 70.52, 70.24, 61.92, 45.19, 32.69, 29.58, 26.83, 25.56 ppm; HRMS (ESI) m/z: [M+H]⁺ calcd for C₁₄H₃₀ClO₅, 313.1776; found 313.1772.

### Example 94: 18-Chloro-3,6,9,12-tetraoxaoctadecyl 4-methylbenzenesulfonate

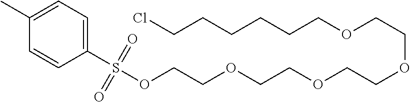 [SMILES: Cc1ccc(S(=O)(=O)OCCOCCOCCOCCOCCCCCCCl)cc1]

A solution of 18-chloro-3,6,9,12-tetraoxaoctadecan-1-ol (1.20 g, 3.84 mmol) in anhydrous pyridine (8.0 mL) under argon atmosphere was cooled to 0° C. in an ice-water bath. After 10 min, p-toluenesulfonyl chloride (1.83 mL, 9.60 mmol) in DCM (5.0 mL) was added dropwise. The reaction mixture was then warmed to room temperature and stirred overnight. Excess pyridine was evaporated under reduced pressure, diluted with 10% citric acid (25.0 mL) and extracted with DCM (2×50.0 mL), the combined extracts were washed with saturated NaCl solution (25.0 mL), dried (Na₂SO₄), filtered, and evaporated under reduced pressure. The resulting residue was purified by flash column chromatography (Silicycle column, 25 g, 20-80% EtOAc/Hexanes, linear gradient for 20 min) to provide 18-chloro-3,6,9,12-tetraoxaoctadecyl 4-methylbenzenesulfonate (1.25 g, 70%) as a colorless oil. ¹H NMR (500 MHz, CDCl₃) δ 7.82-7.77 (m, 2H), 7.36-7.31 (m, 2H), 4.15 (t, J=5.0 Hz, 2H), 3.68 (t, J=5.0 Hz, 2H), 3.65-3.60 (m, 6H), 3.59-3.55 (m, 6H), 3.53 (t, J=6.5 Hz, 2H), 3.45 (t, J=6.5 Hz, 2H), 2.44 (s, 3H), 1.81-1.73 (m, 2H), 1.62-1.55 (m, 2H), 1.48-1.41 (m, 2H), 1.40-1.32 (m, 2H) ppm; ¹³C NMR (125 MHz, CDCl₃) δ 144.91, 133.17, 129.95, 128.13, 71.37, 70.90, 70.77, 70.73, 70.67, 70.24, 69.37, 68.82, 45.20, 32.68, 29.60, 26.83, 25.56, 21.78 ppm; HRMS (ESI) m/z: [M+H]⁺ calcd for C₂₁H₃₆ClO₇S, 467.1865; found 467.1866.

### Example 95: S-(18-Chloro-3,6,9,12-tetraoxaoctadecyl) ethanethioate

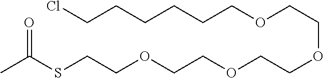 [SMILES: CC(=O)SCCOCCOCCOCCOCCCCCCCl]

A solution of 18-chloro-3,6,9,12-tetraoxaoctadecyl 4-methylbenzenesulfonate (1.25 g, 2.68 mmol) in a mixture of anhydrous THF/DMF (9:1, 25.0 mL) under argon atmosphere was treated with potassium thioacetate (0.30 g, 2.63 mmol). The reaction mixture was then stirred at 55° C. for 12 hours. The reaction mixture was diluted with H₂O (25.0 mL) and extracted with EtOAc (2×50.0 mL), the combined extracts were washed with saturated NaCl solution (25.0 mL), dried (Na2SO4), filtered, and evaporated under reduced pressure. The resulting residue was purified by flash column chromatography (Silicycle column, 25 g, 20-80% EtOAc/Hexanes, linear gradient for 20 min) to provide S-(18-chloro-3,6,9,12-tetraoxaoctadecyl) ethanethioate (0.90 g, 90%) as a color less oil. ¹H NMR (500 MHz, CDCl₃) δ 3.67-3.56 (m, 14H), 3.53 (t, J=6.5 Hz, 2H), 3.45 (t, J=6.5 Hz, 2H), 3.09 (t, J=6.5 Hz, 2H), 2.33 (s, 3H), 1.81-1.73 (m, 2H), 1.63-1.55 (m, 2H), 1.49-1.41 (m, 2H), 1.40-1.33 (m, 2H) ppm; ¹³C NMR (125 MHz, CDCl₃) δ 195.67, 71.38, 70.81, 70.78, 70.76, 70.67, 70.47, 70.26, 69.91, 45.20, 32.69, 30.71, 29.61, 28.99, 26.85, 25.58 ppm; IRMS (ESI) m/z: [M+H]⁺ calcd for C₁₆H₃₂ClO₅S, 371.1653; found 371.1655.

### Example 96: 18-Chloro-3,6,9,12-tetraoxaoctadecane-1-thiol

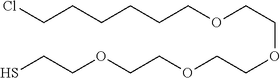 [SMILES: SCCOCCOCCOCCOCCCCCCCl]

A solution of S-(18-chloro-3,6,9,12-tetraoxaoctadecyl) ethanethioate (0.90 g, 2.43 mmol) in anhydrous EtOH (12.5 mL) under argon atmosphere was treated with 12M HCl (0.65 mL). The reaction mixture was then stirred at 90° C. for 14 hours. The reaction mixture was concentrated to ˜2.0 mL and then poured into H₂O (10.0 mL) and extracted with EtOAc (3×25.0 mL), the combined extracts were washed with saturated NH₄Cl solution (40.0 mL), dried (Na₂SO₄), filtered, and evaporated under reduced pressure. The resulting residue was purified by flash column chromatography (Silicycle column, 25 g, 20-80% EtOAc/Hexanes, linear gradient for 20 min) to provide 69 (0.70 g, 88%) as a color less oil. ¹H NMR (500 MHz, CDCl₃) δ 3.67-3.60 (m, 12H), 3.59-3.56 (m, 2H), 3.53 (t, J=6.5 Hz, 2H), 3.45 (t, J=6.5 Hz, 2H), 2.69 (dt, J=8.5, 6.0 Hz, 2H), 1.82-1.73 (m, 2H), 1.63-1.55 (m, 3H), 1.48-1.41 (m, 2H), 1.40-1.33 (m, 2H) ppm; ¹³C NMR (125 MHz, CDCl₃) δ 73.03, 71.38, 70.81, 70.79, 70.78, 70.69, 70.39, 70.26, 45.20, 32.69, 29.61, 26.84, 25.57, 24.42 ppm; IRMS (ESI) m/z: [M+H]⁺ calcd for C₁₄H₃₀ClO₄S, 329.1548; found 329.1545.

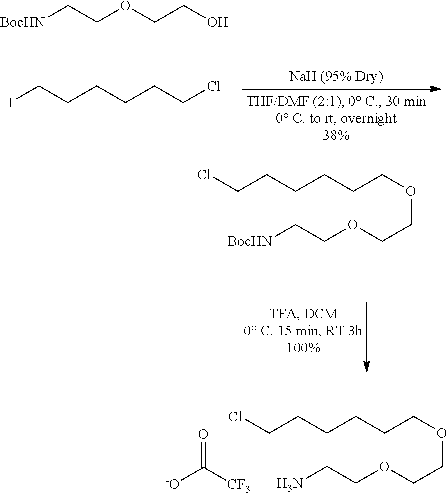

Scheme 9: Synthesis of amine (O2) HaloTag ligand.

### Example 97: tert-Butyl (2-(2-((6-chlorohexyl)oxy)ethoxy)ethyl)carbamate

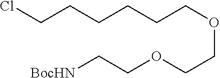 [SMILES: CC(C)(C)OC(=O)NCCOCCOCCCCCCCl]

A solution of tert-butyl (2-(2-hydroxyethoxy)ethyl)carbamate (2.50 g, 12.2 mmol) in a mixture of anhydrous THF/DMF (2:1, 30.0 mL) under argon atmosphere was cooled to 0° C. in an ice-water bath. After 10 min, NaH (dry 95%) (0.35 g, 14.6 mmol) was added. The resulting reaction mixture was stirred at 0° C. for 30 min. At the same temperature, 1-chloro-6-iodohexane (2.80 mL, 18.3 mmol) was added dropwise. The reaction mixture was then warmed to room temperature and stirred overnight. The reaction mixture was then cooled to 5° C. and quenched by addition of NH₄Cl (50.0 mL) and extracted with EtOAc (2×100 mL), the combined extracts were washed with saturated NaCl solution (50.0 mL), dried (Na₂SO₄), filtered, and evaporated under reduced pressure. The resulting residue was purified by flash column chromatography (Silicycle column, 50 g, 0-50% EtOAc/Hexanes, linear gradient for 20 min) to provide tert-Butyl (2-(2-((6-chlorohexyl)oxy)ethoxy)ethyl) carbamate (1.50 g, 38%) as a color less oil. ¹H NMR (500 MHz, CDCl₃) δ 4.99 (br s, 1H), 3.62-3.58 (m, 2H), 3.57-3.52 (m, 4H), 3.53 (t, J=7.0 Hz, 2H, overlapping), 3.46 (t, J=7.0 Hz, 2H), 3.35-3.26 (m, 2H), 1.81-1.73 (m, 2H), 1.64-1.55 (m, 2H), 1.50-1.41 (m, 2H), 1.44 (s, 9H, overlapping), 1.40-1.33 (m, 2H) ppm; ¹³C NMR (125 MHz, CDCl₃) δ 156.11, 79.30, 71.41, 70.41, 70.35, 70.17, 45.17, 40.48, 32.67, 29.57, 28.55, 26.82, 25.55 ppm; HRMS (ESI) m/z: [M+H]⁺ calcd for C₁₅H₃₁ClNO₄, 324.1936; found 324.1930.

### Example 98: 2-(2-((6-Chlorohexyl)oxy)ethoxy)ethan-1-amine

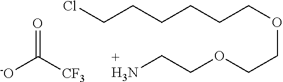

A solution of tert-Butyl (2-(2-((6-chlorohexyl)oxy)ethoxy)ethyl) carbamate (1.50 g, 4.63 mmol) in anhydrous DCM (30.0 mL) under argon atmosphere was cooled to 0° C. in an ice-water bath. After 10 min, TFA (5.0 mL) was added. The resulting reaction mixture was stirred at room temperature for 4 hours, and solvents were evaporated under reduced pressure. The resulting residue was purified by flash column chromatography (Silicycle column, 25 g, 0-15% MeOH/DCM, linear gradient for 20 min) to provide TFA salt of Amine (O2) HaloTag (1.50 g, 100%) as a color less oil. ¹H NMR (500 MHz, CDCl₃) δ 8.10 (br s, 2 H), 3.75 (t, J=4.5 Hz, 2H), 3.68-3.64 (m, 2H), 3.59-3.55 (m, 2H), 3.53 (t, J=7.0 Hz, 2H), 3.46 (t, J=7.0 Hz, 2H), 3.16 (t, J=4.5 Hz, 2H), 1.81-1.73 (m, 2H), 1.62-1.54 (m, 2H), 1.49-1.41 (m, 2H), 1.38-1.30 (m, 2H) ppm; ¹³C NMR (125 MHz, CDCl₃) δ 71.40, 70.50, 69.92, 66.73, 45.17, 39.77, 32.59, 29.37, 26.75, 25.41 ppm; ¹⁹F NMR (470 MHz, CDCl₃) δ −75.84 ppm; HRMS (ESI) m/z: [M+H]⁺ calcd for C₁₀H₂₃ClNO₂, 224.1412; found 224.1408.

### Chemical Properties

The compounds of the instant application exhibit superior stability properties when subjected to physiological conditions. For example, Si-dyes containing Si(OH)₂ bridging atoms undergo Tamao oxidation with physiological levels of peroxide in water to yield orange-fluorescent tetramethylrhodamine, and the silanol also tends to form oligomeric siloxanes in aprotic solvents. Thus, the instant compounds are useful due to their stability.

The photophysical properties of the new Si-rhodamines in aqueous buffer were studied. As reference, these properties were compared to the known dimethylsilyl-bridged SiR dye 001. Literature values for 001 are 644/658 nm, ϕ 0.31, and ε 1.1×10⁵.

It was found that the quantum yield of the new dyes were largely unperturbed by Si-modification, and most of the dyes were as bright or brighter than 001. For example, compound 002 is 30% brighter, primarily due to a larger extinction coefficient. Whereas simple alkyl substitution on silicon did not appreciably affect the fluorescence wavelength, the introduction of vinyl and phenyl groups caused additive red-shifts in excitation and emission (Table 3). Compound 007 is the most red-shifted, with an approximately 15 nm red-shift from 001. Compound 006 is both red-shifted and brighter than 001 due to its higher extinction coefficient. Mixed substitution with phenyl, vinyl, and methyl groups gave intermediate effects: Compound 005 dye roughly split the difference between compound 006 and compound 007 dyes, and is brighter than compounds 003, 004, and 007, but dimmer than compound 006. Chloropropyl silane 009 is not red-shifted but is brighter than 001 owing to its larger extinction coefficient (Table 1). The basis for this increase is unclear, but changes of similar magnitude are known in Si-rhodamines with different amine donors and pendant phenyl groups. On the other hand, incorporation of a trifluoropropyl group into the Si-bridge (010) did not red-shift the dye and lowered brightness.

Strain-promoted lowering of the Si σ* energy in cyclic silanes is another approach to red-shift emission. Treatment of 1-1 with cyclohexyl dichlorosilane yielded the expected Si-rhodamine dye (Scheme 1A). However, the reactions with the corresponding five and four-membered ring analogs yielded many side products. The cyclopentyl analog 028 was obtained in good yield through an alternate synthetic pathway (Scheme 4), but the four-membered ring analog could not be isolated by either route. The silacyclopentyl analog is red-shifted compared to 001 and compound 014, consistent with a strain-induced LUMO-lowering effect (Table 3). However, it was found that compound 028 is chemically unstable in solution, degrading from a blue near-IR dye to a red-colored red-fluorescent dye along with other side-products.

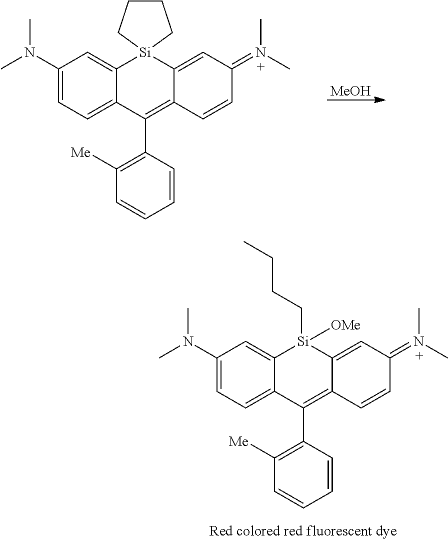

Scheme 10. Instability of silacyclopentyl dye 028 in solution. Proposed ring-opened product in methanol (the solvent used for HRMS analysis).

The effect of divinyl, diphenyl, and chloropropyl silyl groups were evaluated in a broader range of Si-rhodamine dyes, with different amine donors (synthesized using Scheme 3). The previously reported dye compound 016 incorporates rigid tetrahydroquinolines, which red-shifts its fluorescence properties compared to 001. Literature values for SiR680 (016) are 674/689 nm, ϕ 0.35, and ε 1.3×10⁵. b) Literature values for SiR700 (020) are 691/712 nm, ϕ 0.12, and ε 1.0×10⁵. Notably, it was found that the effects of Si-modification are additive within this scaffold, as compounds 017 and 018 dyes are further red-shifted from compound 016 (Table 3). Moreover, compound 017 is 60% brighter than compound 016. Similarly, compounds 021 and 022 indoline dyes are also red-shifted by 12-22 nm, and compound 021 is brighter than the previously reported compound 020. Compound 019 is roughly equivalent to compound 016, whereas compound 023 is slightly brighter. Among these dyes, compound 017 is a particular standout, as it is red-shifted and 60% brighter than 016 (Table 3).

Compared to dimethylsilyl modification, diphenylsilyl modification results in a slight red-shift of excitation and emission in silole optical materials. However, this modification was not known in rhodamines or other green, red, and near-IR dyes used for biological applications, and it was not obvious that the slight red-shift in UV/blue siloles would apply to longer wavelength dyes, or even whether these Si-rhodamines would be stable.

Divinylsilyl substitution has not previously been reported in siloles or any Si-dyes. Furthermore, it was not obvious that it would be a stable or accessible modification, as vinyl silanes have potential reactivity toward both nucleophiles and electrophiles. Remarkably, it was found that this modification is stable and well accommodated in Si-rhodamines. Unexpectedly, it was found that the divinylsilyl dye was both red-shifted and brighter than the dimethylsilyl dye.

Chloropropylsilyl modifications were unknown in Si-rhodamines. Examples of a chloropropyl silyl group in simple siloles exhibits dimmer electroluminescence than the dimethylsilyl modification. Thus, there was no reason to suspect improved Si-rhodamine dye performance, or the ability to tether functionality to the dye, such as described herein. Furthermore, it was not obvious that chloropropyl Si-rhodamines would be stable products, survive the organolithium synthesis conditions, or be capable of being converted to more electrophilic iodides and other functionality without incident.

Incorporation of an electron-rich meta-dimethylaminophenyl group on the silicon quenches fluorescence in a pH-dependent manner. This modification is unknown in Si-dyes and this modification is unknown in any siloles. Although photoinduced electron transfer (PET) quenching behavior is known for electron-rich modifications of the pendant phenyl group of Si-rhodamines, it was not obvious that it would also apply to the Si bridging position, which is further removed from the dye chromophore. This suggests that other PET sensors, including known sensors for calcium, zinc, copper, iron, potassium, nitric oxide, and the like can be fruitfully incorporated onto the bridging silyl group of Si-dyes.

Next began the synthesis of Si-rhodamines containing azetidine electron donors. Azetidines are known to improve the quantum yield of rhodamines compared to dimethylamino groups, e.g., compare 024 (0.47; Table 3) to 001 (0.34; Table 3). Si-modification was well tolerated within this scaffold as well, as it was found that 025 and 027 were both brighter than 024 (Table 3). Furthermore, 026 and 025 are both red-shifted compared to 024 (Table 3). It is expected that other amine donors that improve quantum yields, such as thiomorpholine dioxide, will be similarly compatible.

Symmetrical substitution of rhodamines at both ortho positions of the pendant phenyl can sterically shield nucleophilic attack at the central carbon. As discussed herein, rhodamine dyes with only a single ortho-methyl group on the pendant phenyl ring can be subject to nucleophilic attack at this position. Furthermore, the symmetric substitution will yield only one dye isomer. Therefore, CPM Si-rhodamine dyes in a 2,6-dimethoxy scaffold were synthesized (Table 3). Displacement of the chloride in 030 with iodide formed 046.

Rhodamine dyes that can spirolactonize are valuable for live cell imaging, as the spirolactone form is nonfluorescent and cell permeable, whereas the zwitterionic form is brightly fluorescent and can selectively form when bound to particular target biomolecules. A series of Si-modified Si-rhodamine spirolactones was therefore synthesized. Notable examples include 031 and 032. These dyes exist primarily in the spirolactone form in aqueous buffer, with K_{L-Z} lactone-zwitterion equilibrium values of 0.0034 and 0.002. Estimation of the maximal extinction coefficient when ring-opened has been reported using EtOH/0.1% TFA (Table 3).

Since many dyes that can form spirolactones exist primarily in the spirolactone form in aqueous buffer (e.g., PBS), the compounds poorly absorb light and are poorly fluorescent. Estimation of the maximal extinction coefficient when ring opened has previously been performed in EtOH/0.1% TFA or 0.1% SDS/PBS. Compound 027 was first synthesized as a mix of two isomers, and its maximal extinction coefficient was −215,000 in EtOH/0.1% TFA, versus ˜4,000 when measured in PBS. The excitation wavelength was 653 nm, with emission at 670 nm. The quantum yield in EtOH/0.1% TFA was 0.61. In a subsequent scale-up reaction, it was found that the two isomers of the 035 and 036 lactone dye could be separated.

Two other Si-modified examples in the azetidine series gave relatively low extinction coefficient maximum values in EtOH/0.1% TFA. The values for compound 033 and compound 034 were 80,000 M−1 cm-1, which may indicate that the dyes are still appreciably in the spirolactone form. The open-closed equilibrium of the spirolactone dyes thus appears to depend on nature of the Si-modification, much as the open-closed equilibrium has also been shown to depend on the nature of the amine donor and the acidity of the spirolactone leaving group. These properties could be further tuned as desired. Using anhydrous EtOH/0.1% TFA increased these values to ˜124 000 and 144 000, respectively. Measurement of aqueous quantum yields were not attempted as these spirolactones did not appreciably absorb in PBS alone, and the K_{L-Z} lactone-zwitterion equilibrium value for 034 is 0.00125. Because dimethylamine-donor dyes such as 032 are known to adopt the open zwitterionic form more than azetidine-donor spirolactones like 031 (Scheme 4 and Table 3), and the azetidine in dye 029 is labile to iodide, the chloropropylsilyl modification was evaluated with dimethylamine donors.

The azetidine-donor spirolactone Si-rhodamine dye F646 (031) is known to slightly prefer the spirolactone form, in aqueous buffer with K_{L_Z} lactone-zwitterion equilibrium values of 0.0034 and 0.002, compared to the dimethylamine-donor dye 032. Estimation of the maximal extinction coefficient when ring-opened has been reported using EtOH/0.100 TFA (Table 3). It is apparent to the naked eye that there is some blue color for 032 in PBS compared to 031, suggesting a shift in lactone equilibrium toward the open form.

The synthesis of the Si-chloropropylsilyl analog of 032 yielded two separable isomeric products (037 and 038). Both have a quantum yield of 0.40 in PBS, and favor the open form more than 031. The extinction coefficient in EtOH/0.1% TFA for both is ˜200,000 M−1 cm−1. Overall brightness is ˜80,000 M−1 cm−1, higher than what was measured for 031 (Table 3).

TABLE 3
Photophysical properties of exemplified compounds in PBS

| Compound | | | | | Brightness |
|---|---|---|---|---|---|
| No. | Ex (nm) | Em (nm) | ϕ | ε (M⁻¹ cm⁻¹) | (ϕ × ε) |
| 001 | 648 | 663 | 0.34 | 126,400 | 43,102 |
| 002 | 648 | 664 | 0.36 | 164,900 | 58,540 |
| 003 | 651 | 668 | 0.35 | 141,400 | 48,924 |
| 004 | 657 | 670 | 0.35 | 99,140 | 34,203 |
| 005 | 658 | 676 | 0.36 | 150,700 | 53,499 |
| 006 | 657 | 672 | 0.36 | 158,300 | 56,671 |
| 007 | 663 | 677 | 0.35 | 120,900 | 41,952 |
| 008 | 651 | 668 | 0.02 | 154,200 | 3700 |
| 009 | 650 | 665 | 0.36 | 180,600 | 64,113 |
| 010 | 650 | 666 | 0.35 | 112,900 | 39,854 |
| 011 | 651 | 666 | 0.57 | 156,300 | 89,091 |
| | | | (EtOH) | (EtOH) | (EtOH) |
| 013 | 649 | 666 | 0.38 | 160,000 | 60,800 |
| 014 | 650 | 666 | 0.33 | 170,000 | 56,610 |
| 016 | 674 | 692 | 0.31 | 135,000 | 41,850 |
| 017 | 683 | 702 | 0.32 | 234,700 | 75,104 |
| 018 | 690 | 710 | 0.28 | 148,300 | 41,524 |
| 019 | 677 | 694 | 0.33 | 125,000 | 41,250 |
| 020 | 690 | 715 | 0.17 | 100,000 | 17,000 |
| 021 | 702 | 728 | 0.13 | 136,100 | 17,693 |
| 022 | 710 | 737 | 0.11 | 117,000 | 12,870 |
| 023 | 693 | 717 | 0.17 | 129,100 | 21,947 |
| 024 | 649 | 663 | 0.47 | 118,000 | 55,460 |
| 025 | 663 | 680 | 0.41 | 154,200 | 63,222 |
| 026 | 658 | 673 | 0.44 | 109,700 | 48,268 |
| 027 | 651 | 668 | 0.49 | 166,700 | 81,683 |
| 028 | 653 | 671 | 0.35 | 158,000 | 54,826 |
| 029 | 653 | 670 | 0.48 | 180,000 | 86,400 |
| 030 | 655 | 673 | 0.38 | 200,000 | 76,000 |
| 031 | 646 | 664 | 0.45 | 156,000 | 70,200 |
| 032 | 648 | 667 | 0.38 | 130,000 | 49,400 |
| 033 | 664 | 681 | ND | 80,000 | |
| | (SDS) | (SDS) | | | |
| 034 | 656 | 675 | ND | 80,000 | |
| | (SDS) | (SDS) | | | |
| 035 | 648 | 666 | ND | 183,000 | |
| 036 | 648 | 666 | 0.53 | 213,200 | |
| 037 | 650 | 665 | 0.40 | 200,000 | 80,000 |
| 038 | 650 | 665 | 0.40 | 200,000 | 80,000 |
| 042 | 645 | 665 | 0.45 | 145,800 | |
| 043 | 646 | 665 | 0.45 | 150,700 | |
| 047 | 645 | 665 | 0.43 | 115,000 | |
| 048 | 645 | 665 | 0.45 | 144,000 | |
| 053 | 645 | 666 | 0.43 | 206,700 | |
| 054 | 645 | 665 | 0.44 | 218,400 | |
| 063 | 645 | 665 | 0.45 | 180,600 | |
| 064 | 645 | 665 | 0.45 | 160,400 | |

### Introduction of Lipophilic Modifications

Potentially, long-chain aliphatic groups could be used to recruit dyes to membranes. For example, the dioctyl dye compound 011 is considerably more lipophilic than the other Si-rhodamines. In PBS, compound 011 exhibited weak fluorescence (QY 0. 18, low extinction coefficient) and a pronounced Rayleigh scatter peak at 1 μM concentration. When the solvent was switched to EtOH, bright fluorescence and no scatter peak was observed (Table 3).

### Introduction of Sensors and Functional Handles

A general strategy to develop fluorescent sensors is photo-induced electron transfer (PET). As has been the case for functional handles, such sensors have typically been incorporated into the pendant phenyl group of rhodamines. It was investigated whether a PET sensor could be introduced directly onto the Si atom of a Si-rhodamine. Compound 008 was synthesized (Scheme 1A) and anticipated to be a PET-based pH sensor that would quench dye fluorescence at physiological pH, but become brightly fluorescent at acidic pH. The quantum yield in PBS at pH 7.4 is 0.02 (Table 3), suggesting strong PET quenching when the sensor is not protonated. Conversely, the quantum yield in pH 3 acetate buffer is 0.31, consistent with relief of PET quenching when the sensor is protonated (Table 3).

The ability to add sensors to the Si bridge in addition to the pendant phenyl opens up possibilities to make dual sensors (e.g., one via the pendant phenyl, one via the Si-bridge), as well as targeted sensors with one sensor moiety and one targeting group.

Silyl modification could also be used to introduce handles, such as the norbornene handle depicted in Scheme 2A, for attachment of dyes to sensors or biomolecules. Such functionality has most typically been attached to the pendant aryl ring of rhodamines. Less frequently, one or more of the amine donors has been modified. Norbornene-functionalized dyes, such as that depicted in Scheme 2A, also have significant potential for incorporation into polymeric materials using ring-opening metathesis polymerization (ROMP).

To further explore the possibility of attachment via the bridging silane, compound 009, which contains a chloropropyl handle, was synthesized (Scheme 1A). Interestingly, compound 009 proved to be 50% brighter than 001, owing to its large extinction coefficient (Table 3).

To further functionalize compound 09, the chloride was displaced with iodide to make the iodopropyl dye. This electrophilic dye could potentially be used in subsequent reactions with nucleophiles such as thiols. However, it was found that treatment with the small nucleophile azide resulted in both iodide displacement and reaction at the central carbon of the rhodamine scaffold, a known site of nucleophilic attack. Adopting a more sterically protected, e.g., 2,6-disubstituted, scaffold could potentially prevent this from happening. Chloropropylsilyl dyes 030 and 029 were therefore synthesized in a 2,6-dimethoxy scaffold (Scheme 4), as symmetrical substitution of rhodamines at both ortho positions of the pendant phenyl sterically shields nucleophilic attack at the central carbon. Displacement of the chloride in 030 with iodide formed iodopropylsilyl dye 046, which could be further elaborated to the azide 052 (Scheme 10). Interestingly, however, the azetidine dye 029 was labile to excess iodide, which resulted in tri-iodination via displacement of the chloro group and ringopening of both azetidines (Scheme 11B).

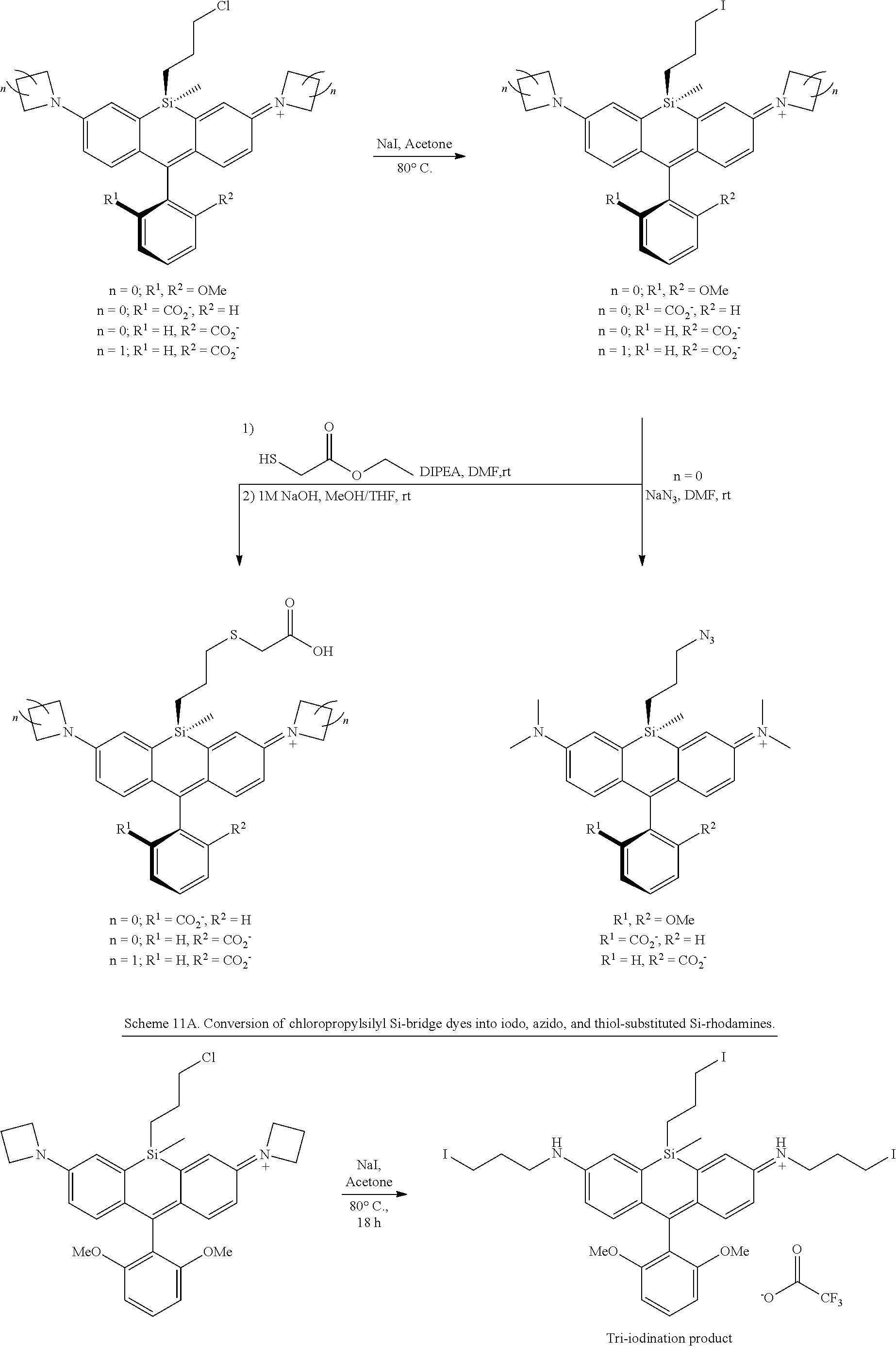

Scheme 11B. Unexpected tri-iodination of compound 029 via displacement of the chloro group and ring-opening of the azetidine rings with iodide. Formation of tri-iodinated product by the treatment of compound 029 with excess NaI.

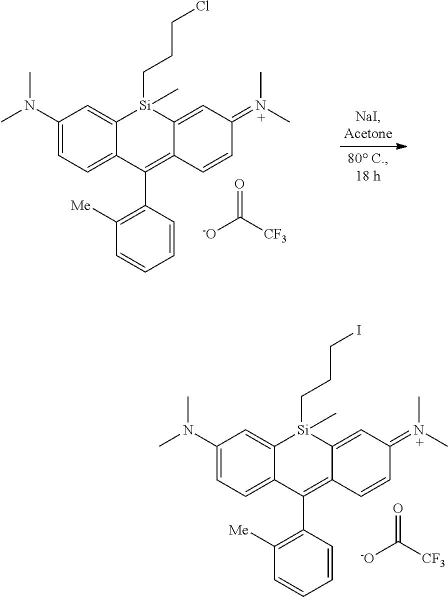

Scheme 11C. Synthesis of compound 067 from corresponding chloropropyl dye 009 with NaI.

No-Wash Live Cell Imaging of the Nucleus with a Si-Bridge Dye

HeLa cells were cultured in Dulbecco's modified Eagle medium (DMEM, from GIBCO, catalog no. 11995065) supplemented with 10% fetal bovine serum (FBS) (GIBCO, catalog no. 10437028) and 1% penicillin-streptomycin (Sigma) at 37° C. in a 5% CO₂ incubator. For imaging, cells were seeded in 35 mm glass bottom dishes (Cellvis, catalog no. D35-28-O—N).

Labeling of the nucleus in live cells using SiR-DNA (Spirochrome, Cytoskeleton cat no. CY-SC007) and compounds 061 and 062 was performed following the manufacturer's instructions for SiR-DNA.

Imaging was performed on a Leica SP-8 Confocal Microscope (SCOPE core facility, UMass Medical School) using a 40×1.30 oil objective. Dyes fluorescing in the Cy5 channel were excited with the HeNe (633 nm) laser at a 15% intensity and detected through a 640-615 band pass filter, and EGFP was excited with the Argon (488 nm) laser. Image analysis was performed using Leica LAS X SP8 software and ImageJ software.

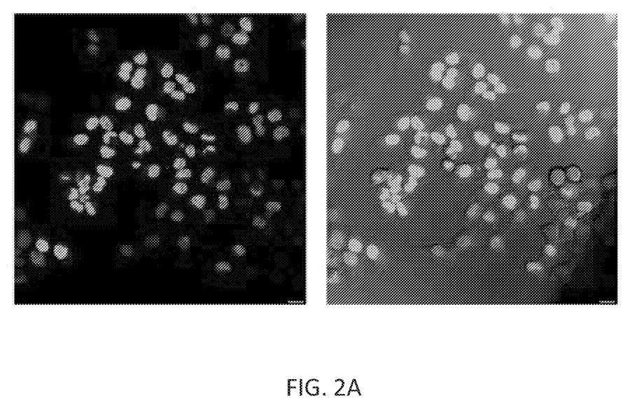

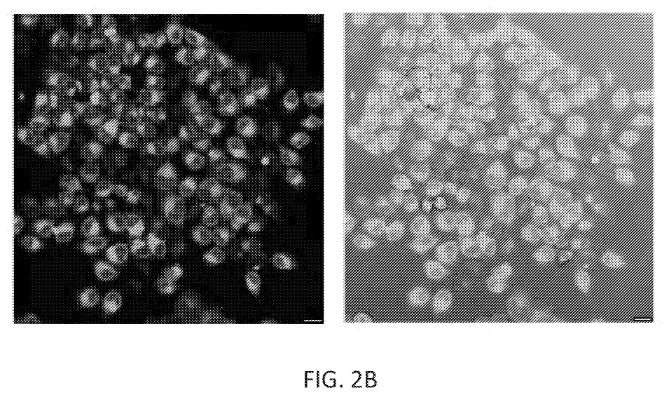

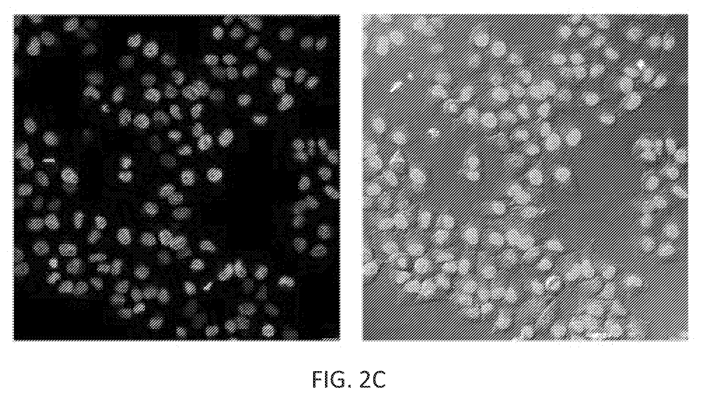

Spirolactonizable Si-rhodamines are particularly valuable for live cell imaging. They typically exist in a nonfluorescent, neutral cell-permeable form that can convert to a highly fluorescent form when bound to particular targets, such as DNA or the protein HaloTag®. This fluorogenic response occurs when the nonfluorescent spirolactone ring opens, generating a zwitterionic dye (Scheme 12). To date, all examples of fluorogenic Si-rhodamine dyes have been modified with targeting groups directly on the pendant phenyl ring that forms the spirolactone. It is not obvious whether this same fluorogenic behavior would also occur with dyes that are modified with targeting groups on the more distal silyl group. SiR-DNA (Spirochrome) is a commercially-available Si-rhodamine dye with the DNA-targeting ligand Hoechst 33258 attached to the pendant phenyl ring (Schemes 13A-C), allowing specific labeling of the nucleus in live cells. Therefore, two Hoechst 33258-modified Si-Bridge isomers were synthesized via direct reaction of the phenol of Hoechst 33258 with the isomeric Si-iodopropyl dyes compounds 047 and 048 (Scheme 6), and then assessed their ability to label the nucleus in live HeLa cells compared to SiR-DNA (FIGS. 2A-2C). It was found that compound 062 is highly fluorogenic, labeling only the nucleus in live cells (FIG. 2C). This importantly demonstrates that the valued fluorogenic effect seen in traditional Si-rhodamine dyes modified on the pendant phenyl ring also translates to Si-rhodamine dyes modified on the silyl group. Interestingly, the isomeric compound 061 yielded no labeling of the nucleus (FIG. 2B). To gain more insight into the molecular basis for this difference, the photophysical properties of compounds 061 and 062 were evaluated in vitro in the presence and absence of a hairpin DNA oligonucleotide (hpDNA) (Table 4). Like SiR-DNA, these dyes are quenched and virtually nonfluorescent in PBS, with quantum yields <0.02 and brightness of <130 M⁻¹ cm⁻¹. In the presence of 30 mM hpDNA, the extinction coefficient of 062 increases to 26 000 M⁻¹ cm⁻¹ and the quantum yield to 0.40, yielding a brightness of 10 400 M⁻¹ cm⁻¹ compared to 8518 M⁻¹ cm⁻¹ for SiR-DNA, whereas 061 exhibits little turn-on response (550 M⁻¹ cm⁻¹). Isomeric dyes modified at different ring positions on the pendant phenyl (position 5 or 6) have also been shown to differ in their behavior toward biomolecules, including their interaction with DNA.

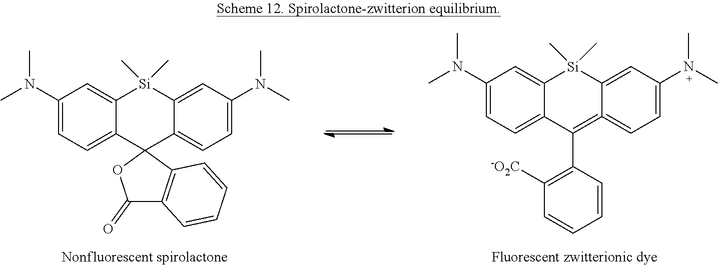

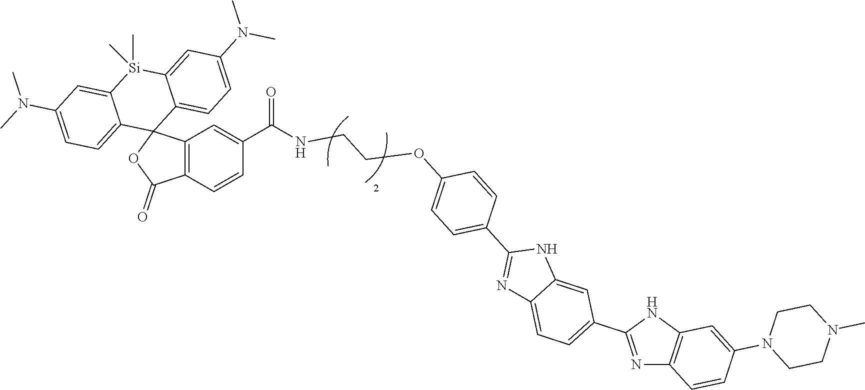

Scheme 13A. Structure of a commercially available fluorogenic Si-rhodamine dye modified with a SiR-DNA targeting group on the pendant phenyl ring.

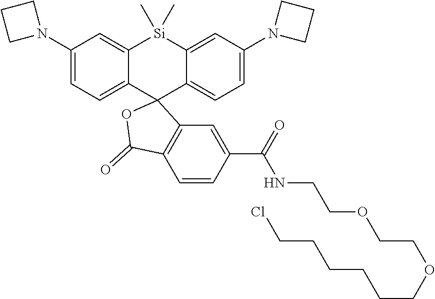 [SMILES: C[Si]1(C)c2cc(N3CCC3)ccc2C2(OC(=O)c3ccc(C(=O)NCCOCCOCCCCCCCl)cc32)c2ccc(N3CCC3)cc21]

Scheme 13B. Structure of a commercially available fluorogenic Si-rhodamine dye modified with a JF646-HaloTag ligand targeting group on the pendant phenyl ring.

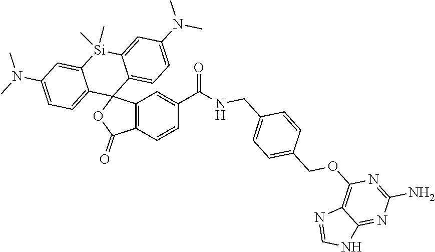 [SMILES: CN(C)c1ccc2c(c1)[Si](C)(C)c1cc(N(C)C)ccc1C21OC(=O)c2ccc(C(=O)NCc3ccc(COc4nc(N)nc5[nH]cnc45)cc3)cc21]

Scheme 13C. Structure of a commercially available fluorogenic Si-rhodamine dye modified with a SNAP-Cell 647-SiR targeting group on the pendant phenyl ring.

Table 4. Photophysical properties of 061 and 062 compared to SiR-DNA.

TABLE 4
Photophysical properties of 061 and 062 compared to SiR-DNA.

| Hoechst Probe | | | ε | | Brightness |
|---|---|---|---|---|---|
| (PBS) | λₑₓ (nm) | λₑₘ (nm) | (M⁻¹cm⁻¹) | ϕ | (ε × ϕ) |
| SiR-DNA | 667 | 673 | 14,500 | 0.003 | 43.5 |
| 061 | 660 | — | 6,500 | 0.02 | 130 |
| 062 | 663 | — | 10,000 | 0.009 | 90 |
| +30 μM hpDNA | | | | | |
| SiR-DNA | 654 | 677 | 54,600 | 0.156 | 8,518 |
| 061 | 658 | 672 | 5,500 | 0.10 | 550 |
| 062 | 653 | 670 | 26,000 | 0.40 | 10,400 |
| 0.1% SDS/PBS | | | | | |
| SiR-DNA | 652 | 676 | 90,000 | 0.524 | 47,160 |
| 061 | 650 | 668 | 25,500 | 0.43 | 10,965 |
| 062 | 649 | 670 | 127,000 | 0.51 | 64,770 |

No-Wash Live Cell Imaging of HaloTag®-Expressing Cells with a Si-Bridge Dye

HeLa cells were seeded in 35 mm glass bottom dishes (Cellvis, catalog no. D35-28-O—N), and transfected with pHaloTag®-EGFP (Addgene #86629). Transient transfections were performed using Lipofectamine 2000 (Invitrogen, catalog no. 1168019) following the manufacturer's instructions. HeLa cell labeling and confocal imaging were performed 24 hr after transfection.

Labeling with JF646-HaloTag® ligand (Promega) and new dyes containing HaloTag® ligands were performed following the manufacturer's instructions. Briefly, cells were incubated with 200 nM dye in DMEM for 15 min at 37° C. Subsequently, images were obtained on a Leica SP-8 Confocal Microscope (SCOPE core facility, UMass Medical School), using a 40×1.15 Oil DIC objective. EGFP was excited with the Argon (488 nm) laser at a 15% intensity and detected through a 505-530 band pass filter and a pinhole set to 53.12 μm. Dyes fluorescing in the Cy5 channel were excited with the HeNe (633 nm) laser at a 15% intensity and detected through a 640-615 band pass filter and 53.12 m pinhole. Image analysis was performed using Leica LAS X SP8 software and ImageJ software.

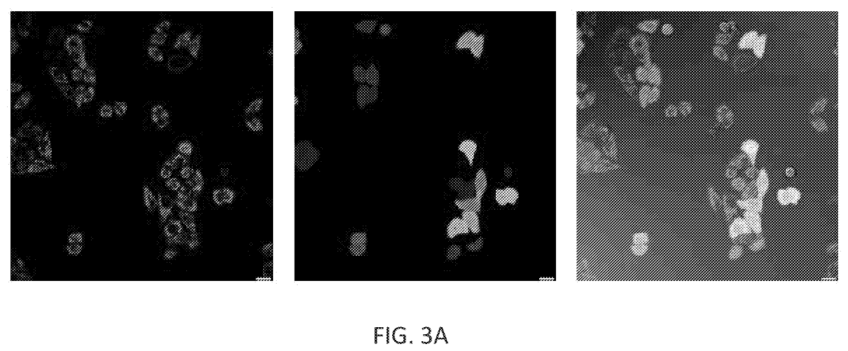

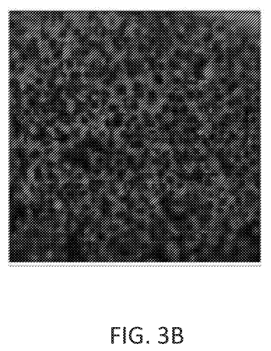

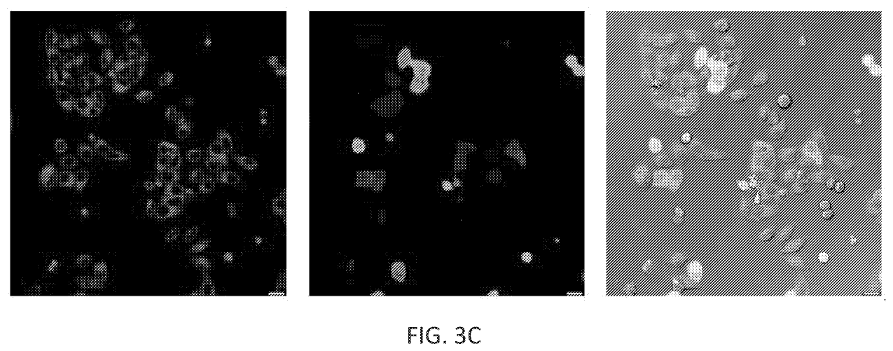

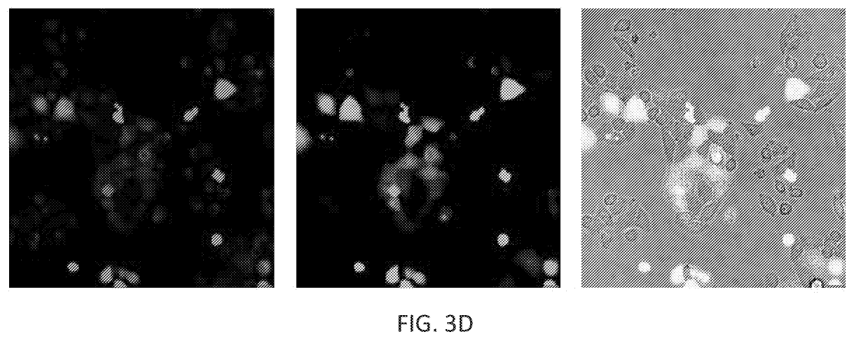

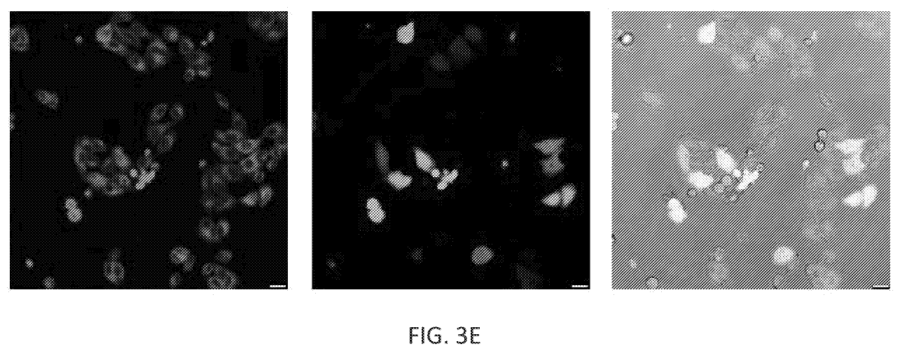

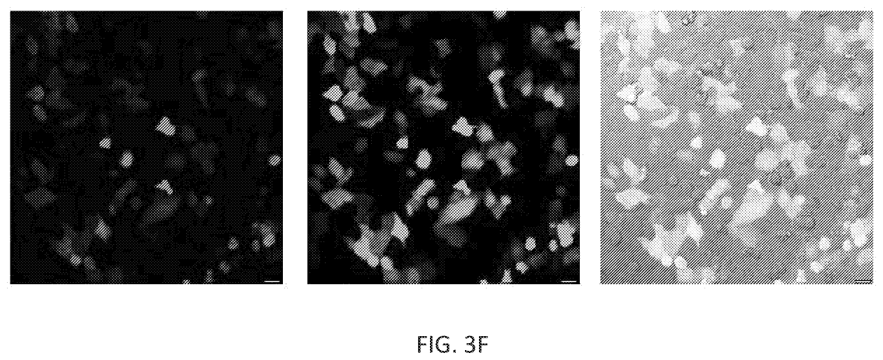

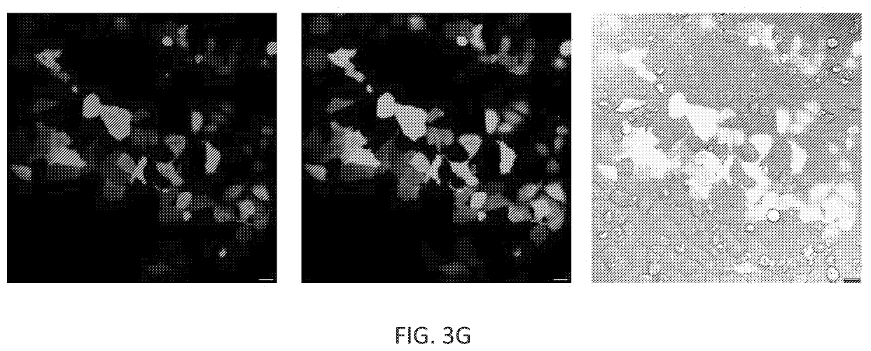

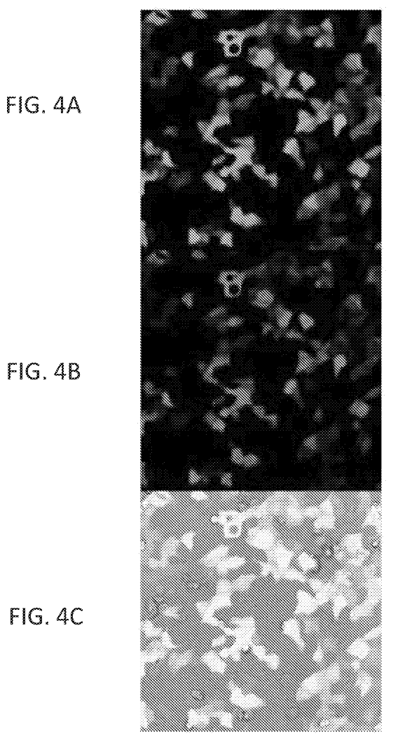

The fluorogenic behavior of compound 062 for DNA labeling suggests that fluorogenic probes for other valuable classes of live cell targets can be developed. Like DNA, the HaloTag® protein presents an anionic surface that is known to favor spirolactone dye ring-opening. While not wishing to be bound to a particular theory, a hypothesis is that favorable interaction with the ring-opened cationic dye occurs only when the anionic carboxylate is facing away from the anionic surface. Thus, it is predicted that fluorogenic Si-bridge dyes targeting HaloTag® will also favor the isomer with the Si-tether and the carboxylate on opposite faces of the dye. To test this supposition, HeLa cells were transfected with pHaloTag®-GFP and treated with 200 nM of five Si-bridge dyes modified with chloroalkane ligands for HaloTag® (compounds 049, 050, 051, 044, and 045 in Scheme 5A). As expected, 050 and 044 did not label HaloTag®-expressing cells, whereas their respective isomers 051 and 045 did (FIGS. 3A-3F). In particular, compound 045 gave bright high contrast images that correlated strongly to GFP expression (FIGS. 4A-4C), whereas compound 044 was poorly fluorescent in all cells (FIGS. 3A-3F). Compound 051 also labeled HaloTag®-expressing cells but had significant background (FIGS. 3A-3F), indicating that the ring-opening equilibrium for this dye is shifted to the more open form. Compound 050 also had high background but did not label HaloTag®-expressing cells. It was anticipated that the spirolactone equilibrium of compound 051 could be tuned to give high contrast images as observed for compound 045 and JF646-HaloTag® ligand (Promega), shown for comparison (FIGS. 3A-3F). The symmetric cationic dye compound 049 only stained mitochondria and did not label HaloTag®-expressing cells, suggesting that sequestration by the negatively polarized mitochondrial membrane dominates its behavior in cells.

Live Cell Imaging of SNAP-Tag-Expressing Cells with Si-Bridge Dyes

HeLa cells were seeded in 35 mm glass bottom dishes (Cellvis, catalog no. D35-28-O—N), and transfected with pSNAPf-H2B control plasmid (Addgene #101124). Transient transfections were performed using Lipofectamine 2000 (Invitrogen, catalog no. 1168019) following the manufacturer's instructions. HeLa cell labeling and confocal imaging were performed 24 hr after transfection.

Labeling with SNAP-Cell® 647 SiR (New England Biolabs, catalog no. S9102S) and new dyes 065 and 066 containing benzylguanine SNAP-tag ligands were performed following the manufacturer's instructions. SNAP-tag is another popular system for labeling fusion proteins. SNAP-tag is more promiscuous towards its substrates than HaloTag, and thus it was anticipated that the relative fluorogenic behavior for each Si-bridge dye isomer would differ from the stark facial selectivity results seen above with Hoechst probes and HaloTag. Briefly, cells were incubated with 3 μM dye in cell culture medium for 30 min at 37° C. The cells were then washed three times with tissue culture medium and incubated in fresh medium for 30 minutes. The medium was replaced one more time to remove unreacted SNAP-tag before imaging.

Images were obtained on a Leica SP-8 Confocal Microscope (SCOPE core facility, UMass Medical School), using a 40×1.15 Oil DIC objective. Dyes fluorescing in the Cy5 channel were excited with the HeNe (633 nm) laser at a 15% intensity and detected through a 640-615 band pass filter and 53.12 m pinhole. Image analysis was performed using Leica LAS X SP8 software and ImageJ software.

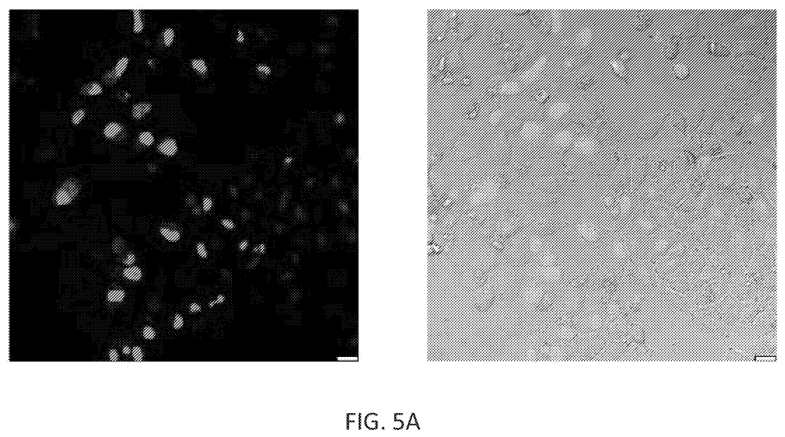

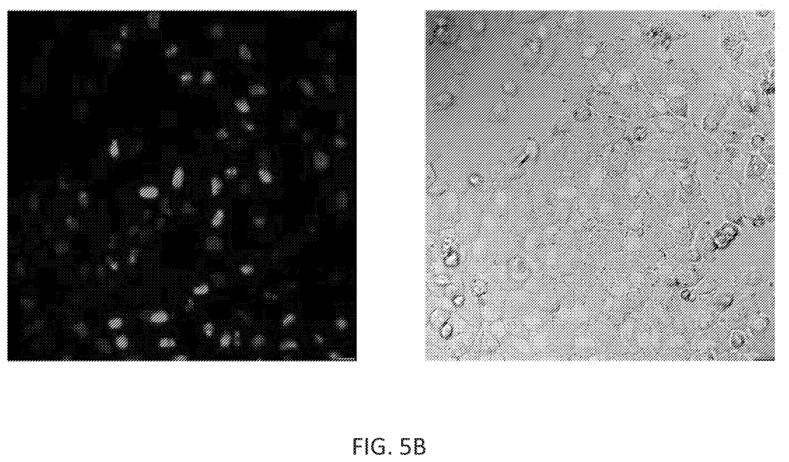

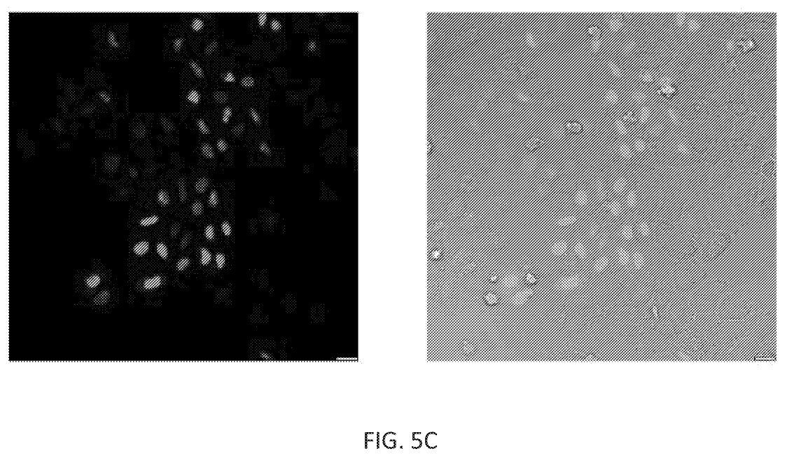

SNAP-tag presents a surface that is less anionic than the HaloTag® protein, and expected to differ in spirolactone dye ring-opening. While not wishing to be bound to a particular theory, a hypothesis is that favorable interaction with SNAP-tag will be less sensitive to whether the anionic carboxylate is facing toward or away from the surface than HaloTag®. To test this supposition, HeLa cells were transfected with pSNAPf-H2B control plasmid and treated with 3 μM dye (SNAP-Cell® 647 SiR or compounds 065, 066 in Table 1 and Scheme 7). Unlike the results with DNA and HaloTag®, both Si-bridge dye isomers labeled SNAP-tag (FIGS. 5A-5C).

The foregoing results were also reported, at least in part, in Chem. Sci., 2022, 13, 6081, and its Supplementary Information, the entire contents of which are incorporated herein by reference.

### REFERENCES

(1) Yamaguchi, S.; Jin, R.-Z.; Tamao, K. Modification of the Electronic Structure of Silole by the Substituents on the Ring Silicon. Journal of Organometallic Chemistry 1998, 559 (1), 73-80. https://doi.org/10.1016/S0022-328X(98)00425-2.
(2) Fu, M.; Xiao, Y.; Qian, X.; Zhao, D.; Xu, Y. A Design Concept of Long-Wavelength Fluorescent Analogs of Rhodamine Dyes: Replacement of Oxygen with Silicon Atom. Chem. Commun. 2008, No. 15, 1780-1782. https://doi.org/10.1039/B718544H.
(3) Koide, Y.; Urano, Y.; Hanaoka, K.; Terai, T.; Nagano, T. Evolution of Group 14 Rhodamines as Platforms for Near-Infrared Fluorescence Probes Utilizing Photoinduced Electron Transfer. ACS Chem. Biol. 2011, 6 (6), 600-608. https://doi.org/10.1021/cb1002416.
(4) Wang, T.; Zhao, Q.-J.; Hu, H.-G.; Yu, S.-C.; Liu, X.; Liu, L.; Wu, Q.-Y. Spirolactonized Si-Rhodamine: A Novel NIR Fluorophore Utilized as a Platform to Construct Si-Rhodamine-Based Probes. Chem. Commun. 2012, 48 (70), 8781-8783. https://doi.org/10.1039/C2CC34159J.
(5) Lukinavicius, G.; Umezawa, K.; Olivier, N.; Honigmann, A.; Yang, G.; Plass, T.; Mueller, V.; Reymond, L.; Corrêa Jr, I. R.; Luo, Z.-G.; Schultz, C.; Lemke, E. A.; Heppenstall, P.; Eggeling, C.; Manley, S.; Johnsson, K. A Near-Infrared Fluorophore for Live-Cell Super-Resolution Microscopy of Cellular Proteins. Nature Chemistry 2013, 5 (2), 132-139. https://doi.org/10.1038/nchem.1546.
(6) Myochin, T.; Hanaoka, K.; Iwaki, S.; Ueno, T.; Komatsu, T.; Terai, T.; Nagano, T.; Urano, Y. Development of a Series of Near-Infrared Dark Quenchers Based on Si-Rhodamines and Their Application to Fluorescent Probes. J. Am. Chem. Soc. 2015, 137 (14), 4759-4765. https://doi.org/10.1021/jacs.5b00246.
(7) Ikeno, T.; Nagano, T.; Hanaoka, K. Silicon-Substituted Xanthene Dyes and Their Unique Photophysical Properties for Fluorescent Probes. Chemistry—An Asian Journal 12 (13), 1435-1446. https://doi.org/10.1002/asia.201700385.
(8) Kolmakov, K.; Hebisch, E.; Wolfram, T.; Nordwig, L. A.; Wurm, C. A.; Ta, H.; Westphal, V.; Belov, V. N.; Hell, S. W. Far-Red Emitting Fluorescent Dyes for Optical Nanoscopy: Fluorinated Silicon-Rhodamines (SiRF Dyes) and Phosphorylated Oxazines. Chemistry—A European Journal 21 (38), 13344-13356. https://doi.org/10.1002/chem.201501394.
(9) Grimm, J. B.; Brown, T. A.; Tkachuk, A. N.; Lavis, L. D. General Synthetic Method for Si-Fluoresceins and Si-Rhodamines. ACS Cent. Sci. 2017, 3 (9), 975-985. https://doi.org/10.1021/acscentsci.7b00247.
(10) Choi, A.; Miller, S. C. Silicon Substitution in Oxazine Dyes Yields Near-Infrared Azasiline Fluorophores That Absorb and Emit beyond 700 Nm. Org. Lett. 2018, 20 (15), 4482-4485. https://doi.org/10.1021/acs.orglett.8b01786.
(11) Pengshung, M.; Neal, P.; L. Atallah, T.; Kwon, J.; R. Caram, J.; A. Lopez, S.; M. Sletten, E. Silicon Incorporation in Polymethine Dyes. Chemical Communications 2020, 56 (45), 6110-6113. https://doi.org/10.1039/C9CC09671J.
(12) Zhou, X.; Lesiak, L.; Lai, R.; Beck, J. R.; Zhao, J.; Elowsky, C. G.; Li, H.; Stains, C. I. Chemoselective Alteration of Fluorophore Scaffolds as a Strategy for the Development of Ratiometric Chemodosimeters. Angew. Chem. Int. Ed. Engl. 2017, 56 (15), 4197-4200. https://doi.org/10.1002/anie.201612628.
(13) Chen, J.; Law, C. C. W.; Lam, J. W. Y.; Dong, Y.; Lo, S. M. F.; Williams, I. D.; Zhu, D.; Tang, B. Z. Synthesis, Light Emission, Nanoaggregation, and Restricted Intramolecular Rotation of 1,1-Substituted 2,3,4,5-Tetraphenylsiloles. Chem. Mater. 2003, 15 (7), 1535-1546. https://doi.org/10.1021/cm021715z.
(14) Son, H.-J.; Han, W.-S.; Chun, J.-Y.; Lee, C.-J.; Han, J.-I.; Ko, J.; Kang, S. O. Spiro-Silacycloalkyl Tetraphenylsiloles with a Tunable Exocyclic Ring: Preparation, Characterization, and Device Application of 1,1′-Silacycloalkyl-2,3,4,5-Tetraphenylsiloles. Organometallics 2007, 26 (3), 519-526. https://doi.org/10.1021/om060854d.
(15) Cai, Y.; Qin, A.; Tang, B. Z. Siloles in Optoelectronic Devices. J. Mater. Chem. C 2017, 5 (30), 7375-7389. https://doi.org/10.1039/C7TC02511D.
(16) Zhou, X.; Lai, R.; Beck, J. R.; Li, H.; Stains, C. I. Nebraska Red: A Phosphinate-Based near-Infrared Fluorophore Scaffold for Chemical Biology Applications. Chem. Commun. (Camb.) 2016, 52 (83), 12290-12293. https://doi.org/10.1039/c6cc05717a.
(17) Son, H.-J.; Han, W.-S.; Chun, J.-Y.; Kwon, S.-N.; Ko, J.; Kang, S. O. Enhanced Charge-Carrier Mobility Derived from Cyclization of a Silanylene Unit on Dithienosiloles: Syntheses, Photophysical Properties, and Device Fabrication of Dithieno-Spiro-Siloles. Organometallics 2008, 27 (11), 2464-2473. https://doi.org/10.1021/om7012088.
(18) Huang, H.; Youn, J.; Ponce Ortiz, R.; Zheng, Y.; Facchetti, A.; Marks, T. Very Large Silacylic Substituent Effects on Response in Silole-Based Polymer Transistors. Chem. Mater. 2011, 23 (8), 2185-2200. https://doi.org/10.1021/cm200009k.
(19) Koide, Y.; Urano, Y.; Hanaoka, K.; Piao, W.; Kusakabe, M.; Saito, N.; Terai, T.; Okabe, T.; Nagano, T. Development of NIR Fluorescent Dyes Based on Si-Rhodamine for in Vivo Imaging. J. Am. Chem. Soc. 2012, 134 (11), 5029-5031. https://doi.org/10.1021/ja210375e.
(20) Miura, T.; Urano, Y.; Tanaka, K.; Nagano, T.; Ohkubo, K.; Fukuzumi, S. Rational Design Principle for Modulating Fluorescence Properties of Fluorescein-Based Probes by Photoinduced Electron Transfer. J Am Chem Soc 2003, 125 (28), 8666-8671.
(21) Devaraj, N. K.; Weissleder, R.; Hilderbrand, S. A. Tetrazine-Based Cycloadditions: Application to Pretargeted Live Cell Imaging. Bioconjugate Chem. 2008, 19 (12), 2297-2299. https://doi.org/10.1021/bc8004446.
(22) Yasir, M.; Liu, P.; Tennie, I. K.; Kilbinger, A. F. M. Catalytic Living Ring-Opening Metathesis Polymerization with Grubbs' Second- and Third-Generation Catalysts. Nat. Chem. 2019, 11 (5), 488-494. https://doi.org/10.1038/s41557-019-0239-4.
(23) Kryman, M. W.; Schamerhorn, G. A.; Yung, K.; Sathyamoorthy, B.; Sukumaran, D. K.; Ohulchanskyy, T. Y.; Benedict, J. B.; Detty, M. R. Organotellurium Fluorescence Probes for Redox Reactions: 9-Aryl-3,6-Diaminotelluroxanthylium Dyes and Their Telluroxides. Organometallics 2013, 32 (15), 4321-4333. https://doi.org/10.1021/om400467s.
(24) Grzybowski, M.; Taki, M.; Senda, K.; Sato, Y.; Ariyoshi, T.; Okada, Y.; Kawakami, R.; Imamura, T.; Yamaguchi, S. A Highly Photostable Near-Infrared Labeling Agent Based on a Phospha-Rhodamine for Long-Term and Deep Imaging. Angewandte Chemie International Edition 2018, 57 (32), 10137-10141. https://doi.org/10.1002/anie.201804731.
(25) Grimm, J. B.; English, B. P.; Chen, J.; Slaughter, J. P.; Zhang, Z.; Revyakin, A.; Patel, R.; Macklin, J. J.; Normanno, D.; Singer, R. H.; Lionnet, T.; Lavis, L. D. A General Method to Improve Fluorophores for Live-Cell and Single-Molecule Microscopy. Nat. Methods 2015, 12 (3), 244-250, 3 p following 250. https://doi.org/10.1038/nmeth.3256.
(26) Lukinavicius, G.; Blaukopf, C.; Pershagen, E.; Schena, A.; Reymond, L.; Derivery, E.; Gonzalez-Gaitan, M.; D'Este, E.; Hell, S. W.; Wolfram Gerlich, D.; Johnsson, K. SiR-Hoechst Is a Far-Red DNA Stain for Live-Cell Nanoscopy. Nature Communications 2015, 6 (1), 8497. https://doi.org/10.1038/ncomms9497.
(27) Bucevicius, J.; Keller-Findeisen, J.; Gilat, T.; W. Hell, S.; Lukinavicius, G. Rhodamine-Hoechst Positional Isomers for Highly Efficient Staining of Heterochromatin. Chemical Science 2019, 10 (7), 1962-1970. https://doi.org/10.1039/C8SC05082A.
(28) Modular Synthetic Approach to Silicon-Rhodamine Homologues and Analogues via Bis-aryllanthanum Reagents. Alexey N. Butkevich Organic Letters 2021 23 (7), 2604-2609
(29) Wang, L., Tran, M., D'Este, E. et al. A general strategy to develop cell permeable and fluorogenic probes for multicolour nanoscopy. Nat. Chem. 12, 165-172 (2020).
(30) R. Wirth, P. Gao, G. U. Nienhaus, M. Sunbul and A. J\ddot{ }aschke, J. Am. Chem. Soc., 2019, 141, 7562-7571.
(31) J. Tyson, K. Hu, S. Zheng, P. Kidd, N. Dadina, L. Chu, D. Toomre, J. Bewersdorf and A. Schepartz, ACS Cent. Sci., 2021, 7, 1419-1426.
(32) S. Uno, M. Kamiya, T. Yoshihara, K. Sugawara, K. Okabe, M. C. Tarhan, H. Fujita, T. Funatsu, Y. Okada, S. Tobita and Y. Urano, Nat. Chem., 2014, 6, 681-689.
(33) Y. Xiao and X. Qian, Coord. Chem. Rev., 2020, 423, 213513.
(34) Y.-L. Huang, A. S. Walker and E. W. Miller, J. Am. Chem. Soc., 2015, 137, 10767-10776.
(35) C. Fischer and C. Sparr, Angew. Chem., Int. Ed., 2018, 57, 2436-2440.
(36) X. Zhou, R. Lai, J. R. Beck, H. Li and C. I. Stains, Chem. Commun., 2016, 52, 12290-12293.
(37) J. Liu, Y.-Q. Sun, H. Zhang, H. Shi, Y. Shi and W. Guo, ACS Appl. Mater. Interfaces, 2016, 8, 22953-22962.
(38) X. Chai, X. Cui, B. Wang, F. Yang, Y. Cai, Q. Wu and T. Wang, Chemistry, 2015, 21, 16754-16758.
(39) H. Nie, L. Qiao, W. Yang, B. Guo, F. Xin, J. Jing and X. Zhang, J. Mater. Chem. B, 2016, 4, 4826-4831.
(40) J. Liu, M. Liu, H. Zhang and W. Guo, Angew. Chem., Int. Ed., 2021, 60, 12992-12998.
(41) D. K. Sharma, S. T. Adams, K. L. Liebmann and S. C. Miller, Org. Lett., 2017, 19, 5836-5839.
(42) X. Lv, C. Gao, T. Han, H. Shi and W. Guo, Chem. Commun., 2020, 56, 715-718.
(43) Q. Zheng, A. X. Ayala, I. Chung, A. V. Weigel, A. Ranjan, N. Falco, J. B. Grimm, A. N. Tkachuk, C. Wu, J. Lippincott-Schwartz, R. H. Singer and L. D. Lavis, ACS Cent. Sci., 2019, 5, 1602-1613.
(44) N. J. Agard, J. A. Prescher and C. R. Bertozzi, J. Am. Chem. Soc., 2004, 126, 15046-15047.
(45) M. F. Debets, S. S. van Berkel, S. Schoffelen, F. P. J. T. Rutjes, J. C. M. van Hest and F. L. van Delft, Chem. Commun., 2010, 46, 97-99.
(46) G. V. Los, L. P. Encell, M. G. McDougall, D. D. Hartzell, N. Karassina, C. Zimprich, M. G. Wood, R. Learish, R. F. Ohana, M. Urh, D. Simpson, J. Mendez, K. Zimmerman, P. Otto, G. Vidugiris, J. Zhu, A. Darzins, D. H. Klaubert, R. F. Bulleit and K. V. Wood, ACS Chem. Biol., 2008, 3, 373-382.
(47) A. Keppler, S. Gendreizig, T. Gronemeyer, H. Pick, H. Vogel and K. Johnsson, Nat. Biotechnol., 2003, 21, 86-89.
(48) J. Wilhelm, S. Kühn, M. Tarnawski, G. Gotthard, J. Tünnermann, T. Tänzer, J. Karpenko, N. Mertes, L. Xue, U. Uhrig, J. Reinstein, J. Hiblot and K. Johnsson, Biochemistry, 2021, 60, 2560-2575.
(49) M. Ebner, I. Lučić, T. A. Leonard and I. Yudushkin, Mol. Cell, 2017, 65, 416-431.e6.
(50) H. Ogasawara, Y. Tanaka, M. Taki and S. Yamaguchi, Chem. Sci., 2021, 12, 7902-7907.
(51) Hanwell, M. D.; Curtis, D. E.; Lonie, D. C.; Vandermeersch, T.; Zurek, E.; Hutchison, G. R. Avogadro: An advanced semantic chemical editor, visualization, and analysis platform. J. Cheminform. 2012, 4, 17.
(52) Grimm, J. B.; Tkachuk, A. N.; Xie, L.; Choi, H.; Mohar, B.; Falco, N.; Schaefer, K.; Patel, R.; Zheng, Q.; Liu, Z.; Lippincott-Schwartz, J.; Brown, T. A.; Lavis, L. D. Nat. Methods 2020, 17, 815-821.
(53) Zhou, X.; Lai, R.; Beck, J. R.; Li, H.; Stains, C. I. Nebraska Red: A Phosphinate-Based near-Infrared Fluorophore Scaffold for Chemical Biology Applications. Chem. Commun. 2016, 52, 12290-12293.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound having the following structural formula: 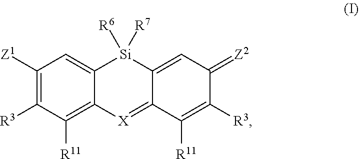
or a tautomer thereof, or a salt of the foregoing, wherein:
X is C-Q or N, and when X is C-Q, each R¹¹ is H, and when X is N, each R¹¹ is independently H, (C₁-C₆)alkyl or halo;
Z¹ and Z² are —N(R¹)(R²); and
R¹ is —H, (C₁-C₆)alkyl, (C₆-C₁₅)aryl or (C₅-C₁₅)heteroaryl; R² is —H, (C₁-C₆)alkyl, (C₆-C₁₅)aryl or (C₅-C₁₅)heteroaryl; and R³ is —H, fluoro, or choro; or
R¹ is —H, (C₁-C₆)alkyl, (C₆-C₁₅)aryl or (C₅-C₁₅)heteroaryl; and R² and R³, taken together with their intervening atoms, form a (C₄-C₈)heterocyclyl; or
R¹ and R², taken together with the N atom to which they are attached, form a (C₃-C₈)heterocyclyl; and R³ is —H, fluoro, or chloro; or
Z¹ is OH and Z² is O; and R³ is —H, fluoro, or chloro;
Q is Ar, (C₁-C₆)alkyl, (C₁-C₆)alkenyl, (C₁-C₆)alkynyl, (C₁-C₆)alkyl-Ar, (C₁-C₆)alkenyl-Ar, (C₁-C₆)alkynyl-Ar, or (C₅-C₁₂)cycloalkenyl-Ar, wherein the alkyl, alkenyl, or alkynyl is optionally substituted with one or more groups independently selected from —P(O)OH(OR⁵⁰), —O—P(O)OH(OR⁵¹), SO₃H, —C(O)NH(R⁴⁰), (C₁-C₆)alkyl-OH, —OH, or —C(O)OH;
Ar is a ring having the structure: 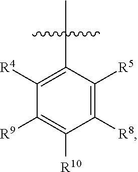
wherein:
R⁴ and R⁵ are each independently —H, —CO₂H, halo, cyano, —OH, (C₁-C₆)alkyl-OH, —SO₃H, nitro, triflate, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl, —C(O)NH(R⁴⁰), —P(O)(OR⁵⁰)₂, —O—P(O)(OR⁵¹)₂, or (C₃-C₈)cyclic amino;
each R⁴⁰ is independently —H, cyano, or SO₂(R⁴¹);
each R⁴¹ is independently (C₁-C₆)alkyl, NH₂, NH((C₁-C₆)alkyl), or N((C₁-C₆)alkyl)₂;
each R⁵⁰ is independently-H, (C₁-C₆)alkyl, or acetoxymethyl;
each R⁵¹ is independently-H, (C₁-C₆)alkyl, or acetoxymethyl;
R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R⁸⁰;
each R⁸⁰ is independently selected from oxo, halo, —CO₂H, —C(O)O—N-succinimide, maleimido, azido, propargyl, norbornenyl or tetrazinyl, or a sensor or targeting group;
R⁹ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; and
R¹⁰ —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R¹⁰⁰⁰;
each R¹⁰⁰ is independently selected from oxo, halo, —CO₂H, —C(O)O—N-succinimide, maleimido, azido, propargyl, norbornenyl, or tetrazinyl, or a sensor or targeting group; or
Ar is a ring having the structure: 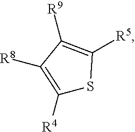
wherein one of R⁴, R⁵, R⁸, and R⁹ is covalently attached to X, and the rest of R⁴, R⁵, R⁸, and R⁹ are as defined above; and
R⁶ is (C₂-C₂₅)aliphatic, (C₂-C₂₅)heteroaliphatic, (C₆-C₁₅)aryl or (C₅-C₁₅)heteroaryl; and
R⁷ is (C₁-C₂₅)aliphatic, (C₂-C₂₅)heteroaliphatic, (C₆-C₁₅)aryl or (C₅-C₁₅)heteroaryl; or
R⁶ and R⁷, taken together with the Si atom to which they are attached, form a (C₅-C₈)silacycle, wherein:
the aliphatic and heteroaliphatic of R⁶ and R⁷, or the silacycle formed by R⁶ and R⁷, taken together with the Si atom to which they are attached, are optionally substituted with one or more R⁶⁰, and the aryl or heteroaryl of R⁶ and R⁷ are optionally substituted with one or more R⁶¹;
each R⁶⁰ is independently selected from oxo, halo, —CO₂H, —C(O)O—N-succinimide, maleimido, amino, hydroxyl, thiol, azido, propargyl, norbornenyl, or tetrazinyl, or a sensor or targeting group; and
each R⁶¹ is independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H.

2. The compound of claim 1, wherein the compound is a compound of Formula (II): 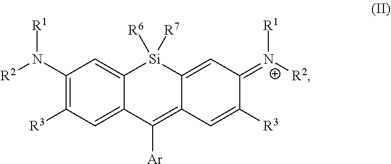
or a tautomer thereof, or a salt of the foregoing.

3. The compound of claim 1, wherein R¹ is (C₁-C₆)alkyl; R² is (C₁-C₆)alkyl; and R³ is H, fluoro, or chloro.

4. (canceled)

5. The compound of claim 1, wherein R¹ is (C₁-C₆)alkyl; and R² and R³, taken together with their intervening atoms, form a (C₄-C₈)heterocyclyl.

6. (canceled)

7. The compound of claim 1, wherein R¹ and R², taken together with the N atom to which they are attached, form aziridinyl or azetidinyl; and R³ is —H, fluoro, or chloro.

8. The compound of claim 1, wherein R⁴ is —H, —CO₂H, (C₁-C₆)alkyl, (C₁-C₆)haloalkyl, (C₁-C₆)alkoxy or (C₁-C₆)haloalkoxy.

9. (canceled)

10. The compound of claim 1, wherein R⁵ is —H, (C₁-C₆)alkyl, (C₁-C₆)haloalkyl, (C₁-C₆)alkoxy or (C₁-C₆)haloalkoxy.

11. (canceled)

12. (canceled)

13. (canceled)

14. The compound of claim 1, wherein R⁴ is —CO₂H and R⁵ is —H.

15. (canceled)

16. The compound of claim 1, wherein R⁶ is optionally substituted (C₂-C₁₅)aliphatic or (C₂-C₁₅)heteroaliphatic.

17. (canceled)

18. (canceled)

19. (canceled)

20. (canceled)

21. The compound of claim 1, wherein R⁶ is ethyl, vinyl, 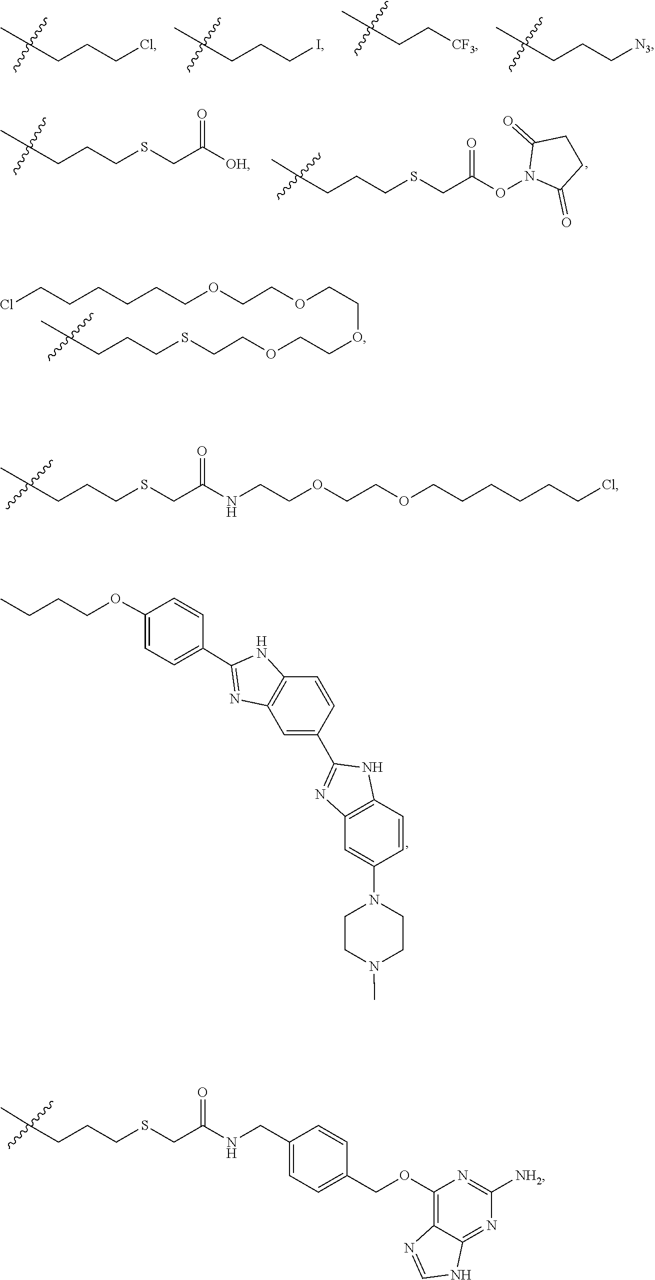
octyl, octadecyl, norbornenyl, phenyl, or dimethylaminophenyl.

22. (canceled)

23. (canceled)

24. (canceled)

25. The compound of claim 1, wherein R⁷ is methyl, ethyl, phenyl, vinyl, octyl or octadecyl.

26. (canceled)

27. (canceled)

28. (canceled)

29. The compound of claim 1, wherein R⁶ and R⁷, taken together with the Si atom to which they are attached, form an optionally substituted (C₅-C₈)silacycle.

30. (canceled)

31. (canceled)

32. (canceled)

33. The compound of claim 1, wherein R⁸ is —H or carboxy.

34.-45. (canceled)

46. The compound of claim 1, having the following structural formula: 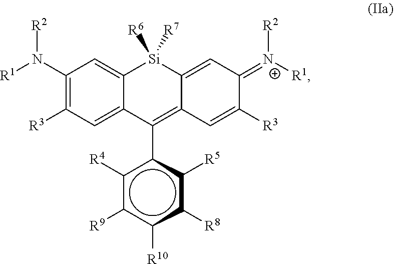
or a stereoisomer or tautomer thereof,
or a salt of the foregoing.

47. (canceled)

48. A method of modifying a compound having the following structural formula: 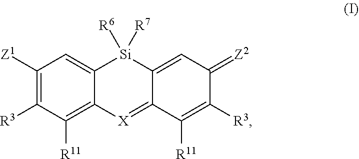
or a tautomer thereof, or a salt of the foregoing, wherein:
X is C-Q or N, and when X is C-Q, each R¹¹ is H, and when X is N, each R¹¹ is independently H, (C₁-C₆)alkyl or halo;
Z¹ and Z² are —N(R¹)(R²); and
R¹ is —H, (C₁-C₆)alkyl, (C₆-C₁₅)aryl or (C₅-C₁₅)heteroaryl; R² is —H, (C₁-C₆)alkyl, (C₆-C₁₅)aryl or (C₅-C₁₅)heteroaryl; and R³ is —H, fluoro, or choro; or
R¹ is —H, (C₁-C₆)alkyl, (C₆-C₁₅)aryl or (C₅-C₁₅)heteroaryl; and R² and R³, taken together with their intervening atoms, form a (C₄-C₈)heterocyclyl; or
R¹ and R², taken together with the N atom to which they are attached, form a (C₃-C₈)heterocyclyl; and R³ is —H, fluoro, or chloro; or
Z¹ is OH and Z² is O; and R³ is —H, fluoro, or chloro;
Q is Ar, (C₁-C₆)alkyl, (C₁-C₆)alkenyl, (C₁-C₆)alkynyl, (C₁-C₆)alkyl-Ar, (C₁-C₆)alkenyl-Ar, (C₁-C₆)alkynyl-Ar, or (C₅-C₁₂)cycloalkenyl-Ar, wherein the alkyl, alkenyl, or alkynyl is optionally substituted with one or more groups independently selected from —P(O)OH(OR⁵⁰), —O—P(O)OH(OR⁵¹), SO₃H, —C(O)NH(R⁴⁰), (C₁-C₆)alkyl-OH, —OH, or —C(O)OH;
Ar is a ring having the structure: 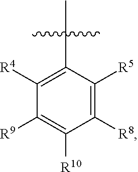
wherein:
R⁴ and R⁵ are each independently —H, —CO₂H, halo, cyano, —OH, (C₁-C₆)alkyl-OH, —SO₃H, nitro, triflate, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl, —C(O)NH(R⁴⁰), —P(O)(OR⁵⁰)₂, —O—P(O)(OR⁵¹)₂, or (C₃-C₈)cyclic amino;
each R⁴⁰ is independently —H, cyano, or SO₂(R⁴¹);
each R⁴¹ is independently (C₁-C₆)alkyl, NH₂, NH((C₁-C₆)alkyl), or N((C₁-C₆)alkyl)₂;
each R⁵⁰ is independently-H, (C₁-C₆)alkyl, or acetoxymethyl;
each R⁵¹ is independently-H, (C₁-C₆)alkyl, or acetoxymethyl;
R⁸ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R⁸⁰;
each R⁸⁰ is independently selected from oxo, halo, —CO₂H, —C(O)O—N-succinimide, maleimido, azido, propargyl, norbornenyl or tetrazinyl, or a sensor or targeting group;
R⁹ is —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino; and
R¹⁰ —H, —CO₂H, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₈)cycloalkyl or (C₃-C₈)cyclic amino, or (C₁-C₂₅)aliphatic or (C₂-C₂₅)heteroaliphatic optionally substituted with one or more R¹⁰⁰;
each R¹⁰⁰ is independently selected from oxo, halo, —CO₂H, —C(O)O—N-succinimide, maleimido, azido, propargyl, norbornenyl, or tetrazinyl, or a sensor or targeting group; or
Ar is a ring having the structure: 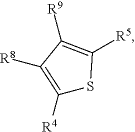
wherein one of R⁴, R⁵, R⁸, and R⁹ is covalently attached to X, and the rest of R⁴, R⁵, R⁸, and R⁹ are as defined above; and
R⁶ is (C₁-C₂₅)aliphatic, (C₂-C₂₅)heteroaliphatic substituted with a leaving group;
R⁷ is (C₁-C₂₅)aliphatic, (C₂-C₂₅)heteroaliphatic, (C₆-C₁₅)aryl or (C₅-C₁₅)heteroaryl,
wherein:
the aliphatic and heteroaliphatic of R⁶ and R⁷ are optionally substituted with one or more R⁶⁰, and the aryl or heteroaryl of R⁷ is optionally substituted with one or more R⁶¹;
each R⁶⁰ is independently selected from oxo, halo, —CO₂H, —C(O)O—N-succinimide, maleimido, amino, hydroxyl, thiol, azido, propargyl, norbornenyl, or tetrazinyl, or a sensor or targeting group; and
each R⁶¹ is independently selected from halo, azido, amino, (C₁-C₆)alkyl, (C₁-C₆)haloalkyl, (C₁-C₆)alkoxy, (C₁-C₆)haloalkoxy, (C₁-C₆)alkylamino, (C₁-C₆)dialkylamino, hydroxyl, thiol or —CO₂H;
the method comprising reacting the compound of Structural Formula I, or a tautomer thereof, or a salt of the foregoing, or an appropriately protected derivative of any of the foregoing, with a nucleophile under conditions suitable for the nucleophile to displace the leaving group, thereby modifying the compound of Structural Formula I, or a tautomer thereof, or a salt of the foregoing.

49. The method of claim 48, wherein the leaving group is iodo or chloro.

50. The method of claim 48, wherein the nucleophile is a thiol, amine, hydroxyl, phosphine, carbanion, sulfinite, azide, cyano, or phosphite.

51. (canceled)

52. A method of imaging a live cell, comprising contacting the cell with a compound of claim 1, or a tautomer thereof, or a salt of the foregoing; illuminating the cell; and detecting fluorescence from the cell.

53. A method of detecting a target in a sample, comprising contacting the sample with a compound of claim 1 comprising a targeting group for the target, or a tautomer thereof, or a salt of the foregoing; illuminating the sample; and detecting fluorescence from the sample.

54. (canceled)

55. A method of labeling a biomolecule or cell, comprising contacting the biomolecule or cell with a compound of claim 1, or a tautomer thereof, or a salt of the foregoing.
